(19) United States
(12) Patent Application Publication
(10) Pub. No.: US 2025/0161241 A1
(43) Pub. Date: May 22, 2025

(54) CYSTEAMIDES, THERAPEUTIC COMPOSITIONS THEREOF, AND RELATED METHODS

(71) Applicants: Boyce Thompson Institute for Plant Research, Inc., Ithaca, NY (US); Cornell University, Ithaca, NY (US)

(72) Inventors: Frank C. Schroeder, Ithaca, NY (US); David Artis, Ithaca, NY (US); Mohammad Arifuzzaman, New York, NY (US); Christopher N. Parkhurst, Brooklyn, NY (US); Tae Hyung Won, Ithaca, NY (US)

(21) Appl. No.: 18/839,493

(22) PCT Filed: Mar. 1, 2023
(86) PCT No.: PCT/US23/63454

§ 371 Date: Aug. 19, 2024

Related U.S. Application Data
(60) Provisional application No. 63/315,664, filed on Mar. 2, 2022.

(51) Int. Cl. A61K 31/16; C07C 317/40; C07C 323/41

(52) U.S. Cl. CPC A61K 31/16; C07C 317/40; C07C 323/41

(57) ABSTRACT

The invention relates to cysteamides, therapeutic compositions containing such cysteamides and methods of using the same.

This application claims priority under 35 U.S.C. § 119(e) to U.S. Provisional Patent Application No. 63/315,664, filed Mar. 2, 2022. The foregoing application is incorporated by reference herein.

### GOVERNMENT SUPPORT

This invention was made with Government support under Grant No. U2CES030167 awarded by the National Institutes of Health. The United States Government has certain rights in the invention.

### FIELD OF THE INVENTION

This invention pertains to the field of small molecule therapeutics and provides therapeutic compositions and pharmacologically active analogs of compounds as well as methods of using the same therapeutically.

### BACKGROUND OF THE INVENTION

Several publications and patent documents are cited throughout the specification in order to describe the state of the art to which this invention pertains. Each of these citations is incorporated herein by reference as though set forth in full.

Secondary metabolites derived from plants, fungi and microbes are among the richest sources of therapeutically useful chemical compounds. For example, in the decade between 2000 and 2010, approximately 50% of all NCEs (new chemical entities) approved by the US FDA for use as human drugs were natural products or derivatives of natural products (Newman et al., J Nat Prod. (2012) 75(3): 311-335).

Recent investigations by the inventors have demonstrated that mammals are an unexpected and rich source of small molecules with diverse biological activities. Meanwhile, as the underlying mechanisms of aging, and a wide range of human health disorders becomes better understood, the need for more selective and efficacious therapeutic and pharmaceutical treatments has never been greater

The present invention addresses these and other related needs.

### SUMMARY OF THE INVENTION

Among other things, the present invention encompasses the inventors' discovery of a family of novel small molecule metabolites produced in mice. The inventors have made important additional discoveries regarding the production and function of these metabolites including: that specific microbiota in the mouse gut that are associated with production of the novel metabolites (and the relevance of gut microbiota to human health and their connection to certain health conditions and diseases); the distribution of the new metabolites within the producing organisms' bodies; and the changing levels of absolute and/or relative production, accumulation or consumption of these metabolites in response to diverse metabolic, dietary and/or environmental stimuli. Based on these discoveries, the inventors have recognized that administering compositions containing the identified metabolites (or analogs thereof) provides a useful strategy to improve the health of animals including mammals and humans and/or to treat certain diseases and disorders.

The emerging field of metabolomics is providing profound insights into the structures and functions of the small molecule metabolites that regulate life processes in all creatures including humans. The inventors have undertaken a comparative metabolomic study in mice (Mus musculus) to evaluate the influence of their diet on the small molecule metabolites produced by their gut microbiome. These efforts have led to the identification of a family of previously undescribed molecules comprising amides of cysteamine.

A particular class of these molecules that was found to be modulated in response to the fiber content of the mouse's diet comprises cysteamides of short chain fatty acids (SFCA's). Without being bound by theory or limiting claims of the present invention, it is believed these SFCA cysteamides constitute an important class of signaling molecules that operate between the gut microbiome and other organ systems within the mouse (and by extension humans). Perhaps most notably, the inventors have found evidence certain cysteamide derivatives efficiently cross the blood brain barrier and that these molecules may provide a useful method to modulate the signaling pathways mediating interactions between the gut microbiome and brain chemistry. By extension, such molecules may provide a useful method to modulate broader physiological and/or cognitive states associated with changes in diet. These discoveries have led to the invention of new therapeutic compositions comprising one or more cysteamides (or related synthetic derivatives and/or analogs) as well as methods of administering such compositions to improve the health or wellbeing of an animal and/or of treating, ameliorating, or curing diseases in an animal, mammal, or a human.

Therefore, in one aspect, the present invention encompasses therapeutic compositions and methods comprising a therapeutically effective amount of one or more cysteamides or derivatives or analogs of such cysteamides. The cysteamides of the instant invention can be used in medicine.

In certain embodiments, provided compositions comprise one or more cysteamides of a C₁₋₄₀ carboxylic acid. In certain embodiments, provided compositions comprise one or more cysteamides of an endogenous acid or an acid produced by the gut microbiome. In certain embodiments, provided compositions comprise one or more cysteamides of a short chain fatty acid. In certain embodiments, provided compositions comprise one or more cysteamides of a bile acid.

In certain embodiments, provided compositions are characterized in that the sulfur atom of the cysteamide is substituted with an alkyl group. In certain embodiments, such compositions are characterized in that the sulfur atom of the cysteamide is substituted with a C₁₋₄₀ alkyl group (referred to herein as S-alkyl cysteamides, or SACAs). In certain embodiments, provided compositions comprise S-methylcysteamides.

In certain embodiments, provided compositions comprise a sulfoxide derivative of a cysteamide (hereafter referred to as “S-oxide cysteamides” or SOCAs). In certain embodiments, provided compositions comprise a SOCA of a C₁₋₄₀ carboxylic acid. In certain embodiments, provided compositions comprise one or more SOCAs of an endogenous acid or an acid produced by the gut microbiome. In certain embodiments, provided compositions comprise one or more SOCAs of a short chain fatty acid. In certain embodiments, provided compositions comprise one or more SOCAs of a bile acid.

In certain embodiments, provided compositions comprise a sulfoxide derivative of a S-alkyl cysteamide (hereafter referred to as “as S-oxide alkyl cysteamides, or SOACAs). In certain embodiments, provided compositions comprise a SOACA of a C₁₋₄₀ carboxylic acid. In certain embodiments, provided compositions comprise one or more SOACAs of an endogenous acid or an acid produced by the gut microbiome. In certain embodiments, provided compositions comprise one or more SOACAs of a short chain fatty acid. In certain embodiments, provided compositions comprise one or more SOACAs of a bile acid. In certain embodiments, provided compositions comprise SOACAs that are enantioenriched at the chiral sulfoxide sulfur atom. In certain embodiments, provided therapeutic compositions comprise sulfoxides of S-methylcysteamides.

In certain embodiments, provided compositions comprise a sulfone derivative of a cysteamide (hereafter referred to as cysteamide sulfones or CASOs). In certain embodiments, provided compositions comprise a CASO of a C₁₋₄₀ carboxylic acid. In certain embodiments, provided compositions comprise one or more CASOs of an endogenous acid or an acid produced by the gut microbiome. In certain embodiments, provided compositions comprise one or more CASOs of a short chain fatty acid. In certain embodiments, provided compositions comprise one or more CASOs of a bile acid.

In certain embodiments, provided therapeutic compositions comprise S-alkyl cysteamide sulfones (SACASOs) derived from a C₁₋₄₀ carboxylic acid. In certain embodiments, provided compositions comprise a SACASO of a C₁₋₄₀ carboxylic acid. In certain embodiments, provided compositions comprise one or more SACASOs of an endogenous acid or an acid produced by the gut microbiome. In certain embodiments, provided compositions comprise one or more SACASOs of a short chain fatty acid. In certain embodiments, provided compositions comprise one or more SACASOs of a bile acid. In certain embodiments, provided therapeutic compositions comprise sulfones of S-methylcysteamides.

In another aspect, the present invention encompasses novel compositions of matter including compositions of novel molecules. While some of the cysteamides described herein are naturally occurring molecules, pure samples of these molecules and, in particular, bulk samples of pure cysteamides free from other biological materials are not found in nature. Additionally, many of the cysteamides and related compounds described above have not been detected in nature, even with the aid of highly sensitive and selective analytical techniques such as HPLC-coupled high resolution mass spectroscopy. As such, many of the compounds described above constitute novel non-natural compositions of matter.

In certain embodiments, the present invention provides pure samples of any of the cysteamides, S-alkyl cysteamides, cysteamide S-oxides, S-alkyl cysteamide S-oxides, cysteamide sulfones, and S-alkyl cysteamide sulfones described above and in the genera and subgenera herein. In certain embodiments, the present invention provides novel compositions comprising bulk quantities of such molecules in substantially pure form.

In another aspect, the present invention encompasses methods of improving the health of an animal or of treating or ameliorating a health disorder in an animal by administering to the animal an effective amount of any one or more of the therapeutic compositions described herein. In certain embodiments, the methods comprise administering such a composition to a mammal. In certain embodiments, the methods comprise administering such a composition to a human. In certain embodiments, the methods reduce and/or inhibit regulatory T cells.

In another aspect, the present invention comprises methods of making therapeutic compositions, the methods comprising formulating an effective amount of one or more purified or synthetically-produced cysteamide derivatives described herein (or a pharmaceutically-acceptable salt, prodrug or derivative thereof) into a therapeutic composition. In certain embodiments, such therapeutic compositions are selected from the group consisting of: an injectable liquid, a tablet, a capsule, a pill, a solution or suspension for oral administration, a solid dosage form for suspension or dissolution into a drinkable- or injectable liquid, a dermal patch, an eye drop, a cream, an ointment, a gel, a powder, a spray, an inhalable composition, a suppository, and a nasal spray.

### BRIEF DESCRIPTION OF THE DRAWINGS

FIG. 1 Shows results of a bioassay demonstrating Antagonistic effects of Bile Acid Methyl Cysteamides on FXR.

FIG. 2 Shows ¹H (600 MHz) nuclear magnetic resonance (NMR) spectrum of BU-MCY in methanol-d₄.

FIG. 3 Shows the double-quantum filtered (dqf) correlation spectroscopy (COSY) spectrum of BU-MCY in methanol-d₄.

FIG. 4 Shows the heteronuclear single quantum coherence (HSQC) spectrum of BU-MCY in methanol-d₄.

FIG. 5 Shows the heteronuclear multiple bond correlation (HMBC) spectrum of BU-MCY in methanol-d₄.

FIG. 6 Shows ¹H (600 MHz) NMR spectrum of BU-MCYO in methanol-d₄.

FIG. 7 Shows the dqfCOSY spectrum of BU-MCYO in methanol-d₄.

FIG. 8 Shows the HSQC spectrum of BU-MCYO in methanol-d₄.

FIG. 9 Shows the HMBC spectrum of BU-MCYO in methanol-d₄.

FIG. 10 Shows ¹H (600 MHz) NMR spectrum of BU-MCYO2 in methanol-d₄.

FIG. 11 Shows the dqfCOSY spectrum of BU-MCYO2 in methanol-d₄.

FIG. 12 Shows the HSQC spectrum of BU-MCYO2 in methanol-d₄.

FIG. 13 Shows the HMBC spectrum of BU-MCYO2 in methanol-d₄.

FIG. 14 Shows ¹H (600 MHz) NMR spectrum of taurobutyric acid in methanol-d₄.

FIG. 15 Shows the dqfCOSY spectrum of taurobutyric acid in methanol-d₄.

FIG. 16 Shows the HSQC spectrum of taurobutyric acid in methanol-d₄.

FIG. 17 Shows the HMBC spectrum of taurobutyric acid in methanol-d₄.

FIG. 18 Shows ¹H (600 MHz) NMR spectrum of CA-MCY in methanol-d₄.

FIG. 19 Shows the dqfCOSY spectrum of CA-MCY in methanol-d₄.

FIG. 20 Shows the HSQC spectrum of CA-MCY in methanol-d₄.

FIG. 21 Shows the HMBC spectrum of CA-MCY in methanol-d₄.

FIG. 22 Shows ¹H (600 MHz) NMR spectrum of CA-MCYO2 in methanol-d₄.

FIG. 23 Shows the dqfCOSY spectrum of CA-MCYO2 in methanol-d₄.

FIG. 24 Shows the HSQC spectrum of CA-MCYO2 in methanol-d₄.

FIG. 25 Shows the HMBC spectrum of CA-MCYO2 in methanol-d₄.

FIG. 26 Shows ¹H (600 MHz) NMR spectrum of CDCA-MCY in methanol-d₄.

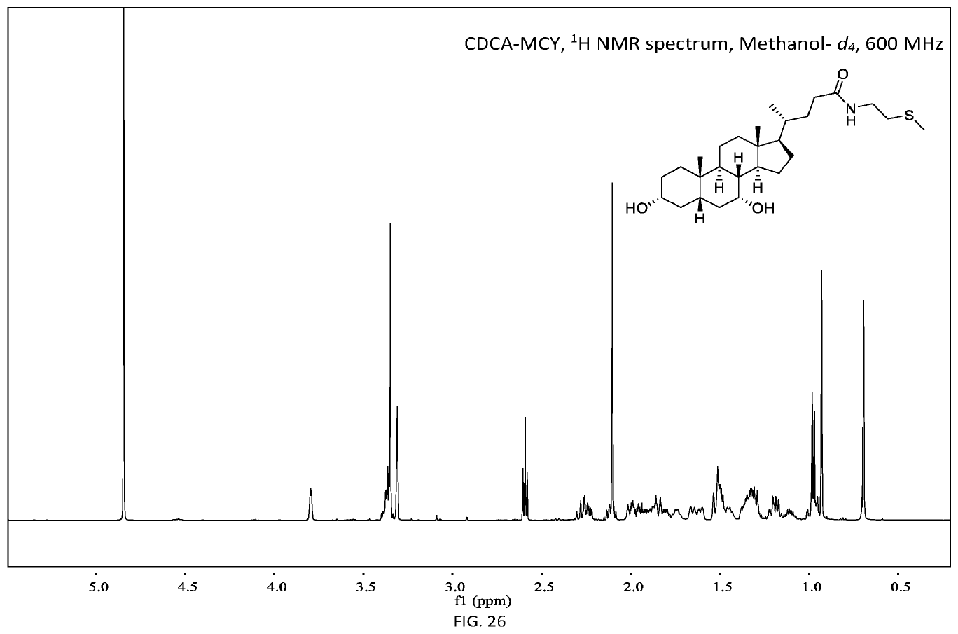

FIG. 27 Shows the dqfCOSY spectrum of CDCA-MCY in methanol-d₄.

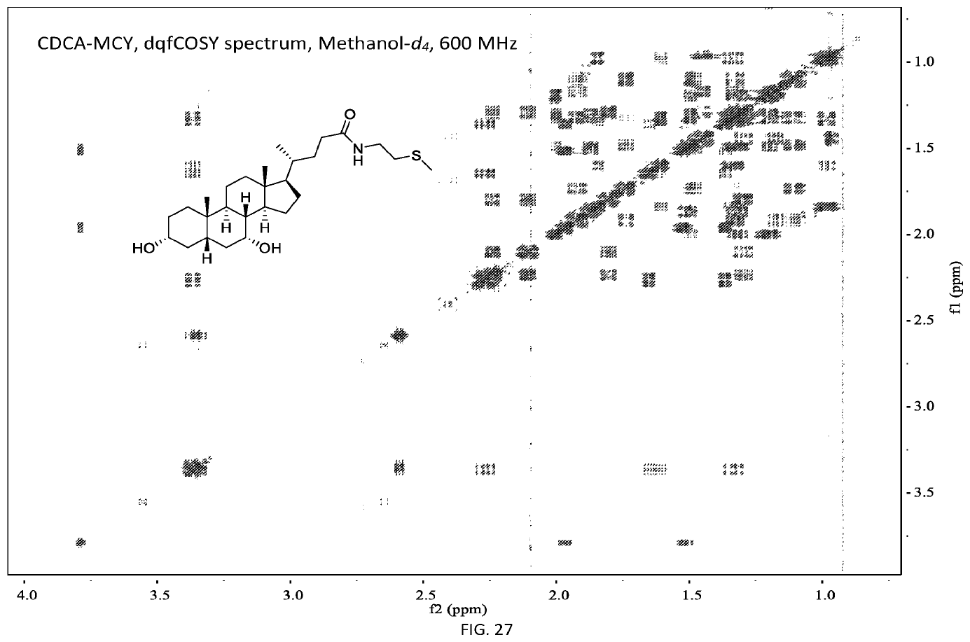

FIG. 28 Shows the HSQC spectrum of CDCA-MCY in methanol-d₄.

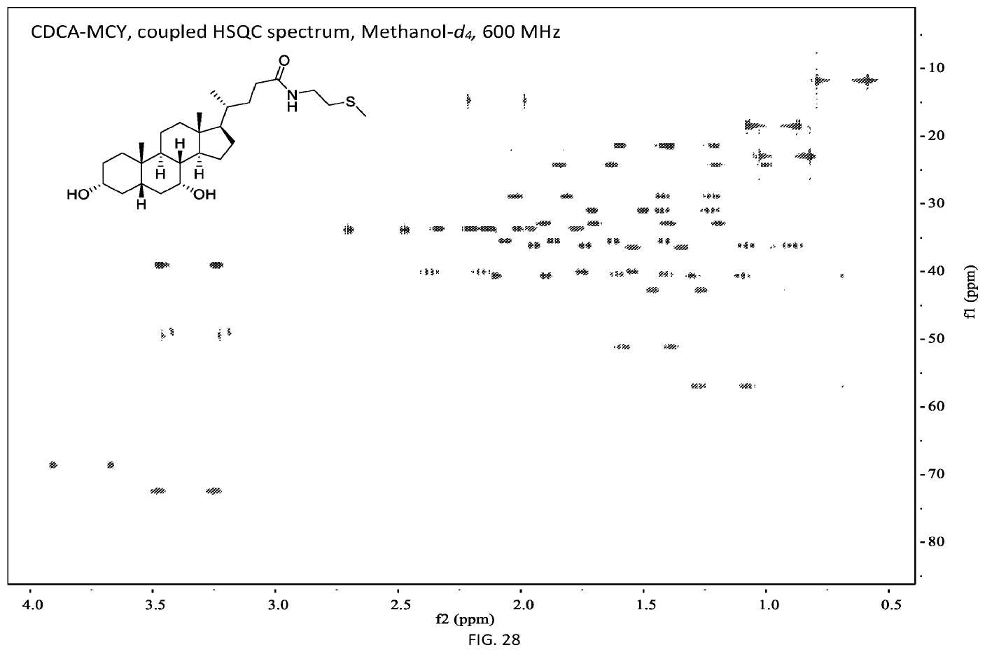

FIG. 29 Shows the HMBC spectrum of CDCA-MCY in methanol-d₄.

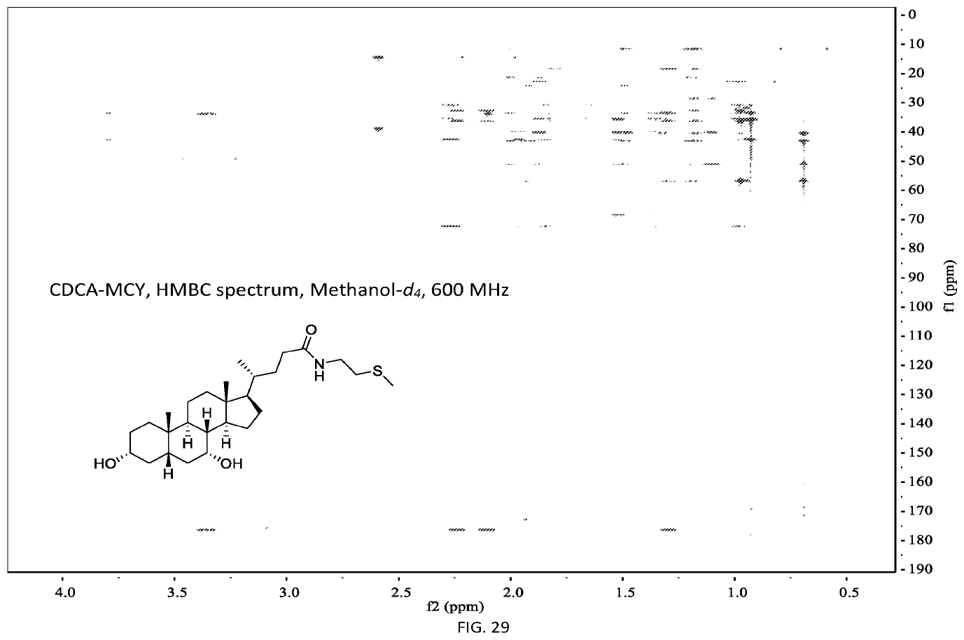

FIG. 30 Shows ¹H (600 MHz) NMR spectrum of CDCA-MCYO in methanol-d₄.

FIG. 31 Shows the dqfCOSY spectrum of CDCA-MCYO in methanol-d₄.

FIG. 32 Shows the HSQC spectrum of CDCA-MCYO in methanol-d₄.

FIG. 33 Shows the HMBC spectrum of CDCA-MCYO in methanol-d₄.

FIG. 34 Shows ¹H (600 MHz) NMR spectrum of UDCA-MCY in methanol-d₄.

FIG. 35 Shows the dqfCOSY spectrum of UDCA-MCY in methanol-d₄.

FIG. 36 Shows the HSQC spectrum of UDCA-MCY in methanol-d₄.

FIG. 37 Shows the HMBC spectrum of UDCA-MCY in methanol-d₄.

FIG. 38 Shows ¹H (600 MHz) NMR spectrum of UDCA-MCYO in methanol-d₄.

FIG. 39 Shows the dqfCOSY spectrum of UDCA-MCYO in methanol-d₄.

FIG. 40 Shows the HSQC spectrum of UDCA-MCYO in methanol-d₄.

FIG. 41 Shows the HMBC spectrum of UDCA-MCYO in methanol-d₄.

FIG. 42 Shows ¹H (600 MHz) NMR spectrum of DCA-MCY in methanol-d₄.

FIG. 43 Shows the dqfCOSY spectrum of DCA-MCY in methanol-d₄.

FIG. 44 Shows the HSQC spectrum of DCA-MCY in methanol-d₄.

FIG. 45 Shows the HMBC spectrum of DCA-MCY in methanol-d₄.

FIG. 46 Shows ¹H (600 MHz) NMR spectrum of DCA-MCYO in methanol-d₄.

FIG. 47 Shows the dqfCOSY spectrum of DCA-MCYO in methanol-d₄.

FIG. 48 Shows the HSQC spectrum of DCA-MCYO in methanol-d₄.

FIG. 49 Shows the HMBC spectrum of DCA-MCYO in methanol-d₄.

FIG. 50 Shows ¹H (600 MHz) NMR spectrum of KDCA-MCY in methanol-d₄.

FIG. 51 Shows the dqfCOSY spectrum of KDCA-MCY in methanol-d₄.

FIG. 52 Shows the HSQC spectrum of KDCA-MCY in methanol-d₄.

FIG. 53 Shows the HMBC spectrum of KDCA-MCY in methanol-d₄.

FIG. 54 Shows ¹H (600 MHz) NMR spectrum of KDCA-MCYO in methanol-d₄.

FIG. 55 Shows the dqfCOSY spectrum of KDCA-MCYO in methanol-d₄.

FIG. 56 Shows the HSQC spectrum of KDCA-MCYO in methanol-d₄.

FIG. 57 Shows the HMBC spectrum of KDCA-MCYO in methanol-d₄.

FIG. 58 Shows ¹H (600 MHz) NMR spectrum of CA-MCYO in methanol-d₄.

FIG. 59 Shows the dqfCOSY spectrum of CA-MCYO in methanol-d₄.

FIG. 60 Shows the HSQC spectrum of CA-MCYO in methanol-d₄.

FIG. 61 Shows the HMBC spectrum of CA-MCYO in methanol-d₄.

FIG. 62 Shows ¹H (600 MHz) NMR spectrum of OMCA-MCY in methanol-d₄.

FIG. 63 Shows the dqfCOSY spectrum of OMCA-MCY in methanol-d₄.

FIG. 64 Shows the HSQC spectrum of OMCA-MCY in methanol-d₄.

FIG. 65 Shows the HMBC spectrum of OMCA-MCY in methanol-d₄.

FIG. 66 Shows biological activity of butyrate-MCY and model for the role of microbiota.

### DETAILED DESCRIPTION OF THE INVENTION

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention can comprise one or more asymmetric centers, and thus can exist in various stereoisomeric forms, e.g., enantiomers and/or diastereomers. Thus, inventive compounds and compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of enantiomers or diastereomers are provided.

Furthermore, certain compounds, as described herein may have one or more double bonds that can exist as either a Z or E isomer, unless otherwise indicated. The invention additionally encompasses the compounds as individual isomers substantially free of other isomers and alternatively, as mixtures of various isomers, e.g., racemic mixtures of enantiomers. In addition to the above-mentioned compounds per se, this invention also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-40 carbon atoms. In certain embodiments, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more double or triple bonds.

The terms “cycloaliphatic”, “carbocycle”, or “carbocyclic”, used alone or as part of a larger moiety, refer to a saturated or partially unsaturated cyclic aliphatic monocyclic, bicyclic, or polycyclic ring systems, as described herein, having from 3 to 12 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons. The terms “cycloaliphatic”, “carbocycle” or “carbocyclic” also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic.

The term “alkyl,” as used herein, refers to saturated, straight- or branched-chain hydrocarbon radicals derived from an aliphatic moiety by removal of a single hydrogen atom. Unless otherwise specified, alkyl groups contain 1-40 carbon atoms. In certain embodiments, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-4o carbon atoms. In certain embodiments, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. Unless otherwise specified, alkynyl groups contain 2-40 carbon atoms. In certain embodiments, alkynyl groups contain 2-12 carbon atoms. In certain embodiments, alkynyl groups contain 2-8 carbon atoms. In certain embodiments, alkynyl groups contain 2-6 carbon atoms. In some embodiments, alkynyl groups contain 2-5 carbon atoms, in some embodiments, alkynyl groups contain 2-4 carbon atoms, in yet other embodiments alkynyl groups contain 2-3 carbon atoms, and in yet other embodiments alkynyl groups contain 2 carbon atoms. Representative alkynyl groups include, but are not limited to, ethynyl, 2-propynyl (propargyl), 1-propynyl, and the like.

The term “carbocycle” and “carbocyclic ring” as used herein, refers to monocyclic and polycyclic moieties wherein the rings contain only carbon atoms. Unless otherwise specified, carbocycles may be saturated, partially unsaturated or aromatic, and contain 3 to 40 carbon atoms or 3 to 20 carbon atoms. Representative carbocyles include cyclopropane, cyclobutane, cyclopentane, cyclohexane, bicyclo[2,2,1]heptane, norbornene, phenyl, cyclohexene, naphthalene, spiro[4.5]decane,

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-14-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In some chemical structures herein, substituents are shown attached to a bond which crosses a bond in a ring of the depicted molecule. This means that one or more of the substituents may be attached to the ring at any available position (usually in place of a hydrogen atom of the parent ring structure). In cases where an atom of a ring so substituted has two substitutable positions, two groups may be present on the same ring atom. When more than one substituent is present, each is defined independently of the others, and each may have a different structure. In certain cases where the substituent shown crossing a bond of the ring is —R, this has the same meaning as if the ring were said to be “optionally substituted” as described in the preceding paragraph.

Suitable monovalent substituents on a substitutable carbon atom of an “optionally substituted” group are independently halogen; —(CH₂)₀₋₄R^{◯}; —(CH₂)₀₋₄OR^{◯}; —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)N(R^{◯})₂; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₄C(O)N(R^{●})₂; —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{◯}₂, —NO₂, —SiR^{◯}₃, —OSiR^{◯}₃, —C(O)SR^{●}, —(C₁₋₄ straight or branched alkylene)C(O)OR^{●}, or —SSR^{◯} wherein each R^{◯} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —^{●}NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†₂, —C(O)R†, —C(O)OR†, —C(O)C(O)R†, —C(O)CH₂C(O)R†, —S(O)₂R†, —S(O)₂NR₂†, —C(S)NR†₂, —C(NH)NR†₂, or —N(R†)S(O)₂R†; wherein each R is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

### Therapeutic Compositions

In one aspect, the present invention encompasses therapeutic compositions comprising a therapeutically effective amount of one or more cysteamides or derivatives or analogs of such cysteamides.

In certain embodiments, provided compositions comprise one or more cysteamides of a C₁₋₄₀ carboxylic acid. In certain embodiments, such compositions comprise a cysteamide of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise cysteamides of straight-chain carboxylic acids. In certain embodiments, such compositions comprise cysteamides of saturated carboxylic acids. In certain embodiments, such compositions are characterized in that they comprise cysteamides of straight-chain saturated carboxylic acids. In certain embodiments, such compositions comprise cysteamides of carboxylic acids that are branched, mono- or poly-unsaturated, optionally substituted, or that have two or more of these features in combination. In certain embodiments, such compositions comprise a cysteamide of an endogenous carboxylic acid. In certain embodiments, such compositions comprise a cysteamide of a microbiome-derived carboxylic acid.

In certain embodiments, provided therapeutic compositions comprise cysteamides derived from a short chain fatty acid. In certain embodiments, such compositions comprise cysteamides of a C₁₋₆ carboxylic acid. In certain embodiments, such compositions comprise a cysteamide of a C₂₋₆ straight chain carboxylic acid. In certain embodiments, such compositions comprise a cysteamide of a C₃₋₆ saturated carboxylic acid. certain embodiments, such compositions comprise a cysteamide derived from a carboxylic acid selected from the group consisting of n-butyric acid, propionic acid, acetic acid and formic acid.

It should be noted that in the preceding paragraph (and in similar contexts throughout this application) the phrase “derived from” as in “derived from a short chain fatty acid” is to be read as defining only the structure of the molecule referred to, and does not refer to the means by which such a molecule is actually made or manufactured. In other words, this language does not imply that the molecule referred to above is required to actually be made or manufactured from short chain fatty acids (though this may also be the case).

In certain embodiments, provided therapeutic compositions are characterized in that the sulfur atom of the cysteamide is substituted with an alkyl group. Such molecules are referred to herein as S-alkyl cysteamides, or SACAs for short. In certain embodiments, the sulfur atom of the SACA is substituted with a C₁₋₄₀ aliphatic group. In certain embodiments, provided compositions are characterized in that the sulfur atom of the cysteamide is substituted with a C₁₋₂₄ alkyl group, a C₁₋₁₂ alkyl group, a C₁₋₈ alkyl group, a C₁₋₆ alkyl group, a C₁₋₅ alkyl group, a C₁₋₄ alkyl group, a C₁₋₃ alkyl group, a C₁₋₂ alkyl group, or with a methyl group. In certain embodiments, such cysteamides are characterized in that they comprise a straight chain alkyl group attached to the sulfur atom. In certain embodiments, such cysteamides are characterized in that they comprise a saturated alkyl group attached to the sulfur atom. In certain embodiments, such cysteamides are characterized in that they comprise a straight-chain saturated alkyl group attached to the sulfur atom. In certain embodiments, such cysteamides are characterized in that the sulfur atom is substituted with a branched alkyl group, a mono- or poly-unsaturated alkyl group, an optionally substituted alkyl group, or an alkyl group having any two or more of these features in combination.

In certain embodiments, provided therapeutic compositions comprise S-alkyl cysteamides (SACAs) derived from a C₁₋₄₀ carboxylic acid. In certain embodiments, such compositions comprise a SACA of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise a SACA of a straight-chain carboxylic acid. In certain embodiments, such compositions comprise a SACA of saturated carboxylic acids. In certain embodiments, such compositions are characterized in that they comprise a SACA of straight-chain saturated carboxylic acid. In certain embodiments, such compositions comprise a SACA of a carboxylic acid that is branched, mono- or poly-unsaturated, optionally substituted, or that has two or more of these features in combination.

In certain embodiments, provided therapeutic compositions comprise SACAs derived from a short chain fatty acid. In certain embodiments, such compositions comprise SACAs of a C₁₋₆ carboxylic acid. In certain embodiments, such compositions comprise a SACA of a C₂₋₆ straight chain carboxylic acid. In certain embodiments, such compositions comprise a SACA of a C₃₋₆ saturated carboxylic acid. In certain embodiments, such compositions comprise a SACA derived from a carboxylic acid selected from the group consisting of n-butyric acid, propionic acid, acetic acid, and formic acid.

In certain embodiments, provided therapeutic compositions comprise a sulfoxide derivative of a cysteamide (hereafter referred to as “S-oxide cysteamides” or SOCAs). In certain embodiments, provided compositions comprise a SOCA derived from a C₁₋₄₀ carboxylic acid. In certain embodiments, provided compositions comprise a SOCA of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise SOCAs of straight-chain carboxylic acids. In certain embodiments, such compositions comprise SOCAs of saturated carboxylic acids. In certain embodiments, such compositions are characterized in that they comprise SOCAs of straight-chain saturated carboxylic acids. In certain embodiments, such compositions comprise SOCAs of carboxylic acids that are branched, mono- or poly-unsaturated, optionally substituted, or that have two or more of these features in combination. In certain embodiments, such compositions comprise a SOCA of an endogenous carboxylic acid. In certain embodiments, such compositions comprise a SOCA of a microbiome-derived carboxylic acid. In certain embodiments, such compositions comprise a SOCA of a bile acid.

In certain embodiments, provided therapeutic compositions comprise SOCAs derived from a short chain fatty acid. In certain embodiments, such compositions comprise SOCAs of a C₁₋₆ carboxylic acid. In certain embodiments, such compositions comprise a SOCA of a C₂₋₆ straight chain carboxylic acid. In certain embodiments, such compositions comprise a SOCA of a C₃₋₆ saturated carboxylic acid. certain embodiments, such compositions comprise a SOCA derived from a carboxylic acid selected from the group consisting of n-butyric acid, propionic acid, acetic acid and formic acid.

In certain embodiments, provided therapeutic compositions are characterized in that the sulfur atom of a provided cysteamide sulfoxide is substituted with an alkyl group. Such molecules are referred to herein as S-oxide alkyl cysteamides, or SOACAs for short. In certain embodiments, the sulfur atom of the SOACA is substituted with a C₁₋₄₀ aliphatic group. In certain embodiments, provided compositions are characterized in that the sulfur atom of the SOACA is substituted with a C₁₋₂₄ alkyl group, a C₁₋₁₂ alkyl group, a C₁₋₈ alkyl group, a C₁₋₆ alkyl group, a C₁₋₅ alkyl group, a C₁₋₄ alkyl group, a C₁₋₃ alkyl group, a C₁₋₂ alkyl group, or with a methyl group. In certain embodiments, such SOACAs are characterized in that they comprise a straight chain alkyl group attached to the sulfur atom. In certain embodiments, such SOACAs are characterized in that they comprise a saturated alkyl group attached to the sulfur atom. In certain embodiments, such SOACAs are characterized in that they comprise a straight-chain saturated alkyl group attached to the sulfur atom. In certain embodiments, such SOACAs are characterized in that the sulfur atom is substituted with a branched alkyl group, a mono- or poly-unsaturated alkyl group, an optionally substituted alkyl group, or an alkyl group having any two or more of these features in combination.

In certain embodiments, provided therapeutic compositions comprise S-oxide alkyl cysteamides (SOACAs) derived from a C₁₋₄₀ carboxylic acid. In certain embodiments, such compositions comprise a SOACA of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise a SOACA of a straight-chain carboxylic acid. In certain embodiments, such compositions comprise SOACAs of saturated carboxylic acids. In certain embodiments, such compositions are characterized in that they comprise a SOACA of a straight chain, saturated carboxylic acid. In certain embodiments, such compositions comprise a SOACA of a carboxylic acid that is branched, mono- or poly-unsaturated, optionally substituted, or that has two or more of these features in combination.

In certain embodiments, such compositions comprise a SOACA of an endogenous carboxylic acid. In certain embodiments, such compositions comprise a SOACA of a microbiome-derived carboxylic acid. In certain embodiments, such compositions comprise a SOACA of a bile acid.

In certain embodiments, provided therapeutic compositions comprise SOACAs derived from a short chain fatty acid. In certain embodiments, such compositions comprise SOACAs of a C₁₋₆ carboxylic acid. In certain embodiments, provided compositions comprise a SOACA of a C₂₋₆ straight chain carboxylic acid. In certain embodiments, provided compositions comprise a SOACA of a C₃₋₆ saturated carboxylic acid. In certain embodiments, provided compositions comprise a SOACA derived from a carboxylic acid selected from the group consisting of n-butyric acid, propionic acid, acetic acid, and formic acid.

The SOACs and SOACAs described above contain a chiral center on the sulfur atom. In certain embodiments, the provided therapeutic compositions comprise an SOAC or an SOACA that is enantioenriched with respect to the chiral sulfur atom. In certain embodiments, such compositions are characterized in that the contained SOACs and/or SOACAs comprise greater than 70% of one stereoisomer of the sulfur-based chiral center. In certain embodiments, such compositions are characterized in that the contained SOACs and/or SOACAs comprise greater than 75%, greater than 80%, greater than 85%, greater than 90%, greater than 92%, greater than 94%, greater than 95%, greater than 96%, greater than 97%, greater than 98%, greater than 99%, or greater than 99.5%, of one stereoisomer of the sulfur-based chiral center. such compositions are characterized in that the contained SOACs and/or SOACAs comprise essentially a single enantiomer with respect to the sulfur-based chiral center. In certain embodiments where the SOAC and/or SOACAs are entantioenriched at the sulfur-based chiral center, the composition is enriched in the compound having the S configuration at the Sulfur-based center. In certain embodiments where the SOAC and/or SOACAs are entantioenriched at the sulfur-based chiral center, the configuration of the dominant enantiomer is R.

In certain embodiments, provided therapeutic compositions comprise a sulfone derivative of a cysteamide (hereafter referred to cysteamide sulfones or CASOs). In certain embodiments, provided compositions comprise a CASO of a C₁₋₄₀ carboxylic acid. In certain embodiments, provided compositions comprise a CASO of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise CASOs of straight-chain carboxylic acids. In certain embodiments, such compositions comprise CASOs of saturated carboxylic acids. In certain embodiments, such compositions are characterized in that they comprise CASOs of straight-chain saturated carboxylic acids. In certain embodiments, such compositions comprise CASOs of carboxylic acids that are branched, mono- or poly-unsaturated, optionally substituted, or that have two or more of these features in combination. In certain embodiments, such compositions comprise a CASO of an endogenous carboxylic acid. In certain embodiments, such compositions comprise a CASO of a microbiome-derived carboxylic acid. In certain embodiments, such compositions comprise a CASO of a bile acid.

In certain embodiments, provided therapeutic compositions comprise CASOs derived from a short chain fatty acid. In certain embodiments, such compositions comprise CASOs of a C₁₋₆ carboxylic acid. In certain embodiments, such compositions comprise a CASO of a C₂₋₆ straight chain carboxylic acid. In certain embodiments, such compositions comprise a CASO of a C₃₋₆ saturated carboxylic acid. In certain embodiments, such compositions comprise a CASO derived from a carboxylic acid selected from the group consisting of n-butyric acid, propionic acid, acetic acid and formic acid.

In certain embodiments, provided therapeutic compositions are characterized in that the sulfur atom of the cysteamide sulfone is substituted with an alkyl group. Such molecules are referred to herein as S-alkyl cysteamide sulfones, or SACASOs for short. In certain embodiments, the sulfur atom of the SACASO is substituted with a C₁₋₄₀ aliphatic group. In certain embodiments, provided compositions are characterized in that the sulfur atom of the SACASO is substituted with a C₁₋₂₄ alkyl group, a C₁₋₁₂ alkyl group, a C₁₋₈ alkyl group, a C₁₋₆ alkyl group, a C₁₋₅ alkyl group, a C₁₋₄ alkyl group, a C₁₋₃ alkyl group, a C₁₋₂ alkyl group, or with a methyl group. In certain embodiments, such SACASOs are characterized in that they comprise a straight chain alkyl group attached to the sulfur atom. In certain embodiments, such SACASOs are characterized in that they comprise a saturated alkyl group attached to the sulfur atom. In certain embodiments, such SACASOs are characterized in that they comprise a straight-chain saturated alkyl group attached to the sulfur atom. In certain embodiments, such SACASOs are characterized in that the sulfur atom is substituted with a branched alkyl group, a mono- or poly-unsaturated alkyl group, an optionally substituted alkyl group, or an alkyl group having any two or more of these features in combination.

In certain embodiments, provided therapeutic compositions comprise S-alkyl cysteamide sulfones (SACASOs) derived from a C₁₋₄₀ carboxylic acid. In certain embodiments, such compositions comprise a SACASO of a C₁₋₂₄ carboxylic acid, a C₁₋₂₀ carboxylic acid, a C₁₋₁₆ carboxylic acid, a C₁₋₁₂ carboxylic acid, a C₁₋₈ carboxylic acid, a C₂₋₁₂ carboxylic acid or a C₂₋₈ carboxylic acid. In certain embodiments, such compositions comprise a SACASO of a straight-chain carboxylic acid. In certain embodiments, such compositions comprise a SACASO of saturated carboxylic acids. In certain embodiments, such compositions are characterized in that they comprise a SACASO of straight-chain saturated carboxylic acid. In certain embodiments, such compositions comprise a SACASO of a carboxylic acid that is branched, mono- or poly-unsaturated, optionally substituted, or that has two or more of these features in combination. In certain embodiments, such compositions comprise a SACASO of an endogenous carboxylic acid. In certain embodiments, such compositions comprise a SACASO of a microbiome-derived carboxylic acid. In certain embodiments, such compositions comprise a SACASO of a bile acid.

In certain embodiments, provided therapeutic compositions comprise SACASOs derived from a short chain fatty acid. In certain embodiments, such compositions comprise SACASO of a C₁₋₆ carboxylic acid. In certain embodiments, provided compositions comprise SACASOs of a C₂₋₆ straight chain carboxylic acid. In certain embodiments, provided compositions comprise a SACASO of a C₃₋₆ saturated carboxylic acid. In certain embodiments, provided compositions comprise a SACASO derived from a carboxylic acid selected from the group consisting of n-butyric acid, propionic acid, acetic acid, and formic acid.

In certain embodiments, the present invention encompasses compositions of matter comprising a therapeutically effective amount of one or more compounds of Formula I:

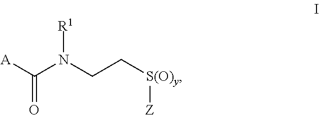

wherein:
   A is selected from —H and an optionally substituted C₁₋₃₉ aliphatic group;
   R¹ is selected from —H, and an optionally substituted C₁₋₄₀ aliphatic group;
   Z is selected from —H, and an optionally substituted C₁₋₄₀ aliphatic group; and
   y is 0, 1, or 2.

In certain embodiments, the present invention encompasses compositions of matter comprising a therapeutically effective amount of one or more compounds of Formula II:

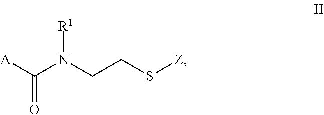

wherein each of A, R¹, and Z is as defined above and in the genera and subgenera herein.

In certain embodiments, the present invention encompasses compositions of matter comprising a therapeutically effective amount of one or more compounds of Formula III:

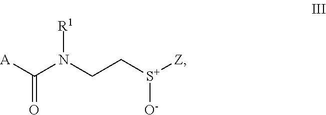

wherein each of A, R¹, and Z is as defined above and in the genera and subgenera herein.

In certain embodiments, the present invention encompasses compositions of matter comprising a therapeutically effective amount of a compound selected from formulae IIIa, IIIb, and IIc:

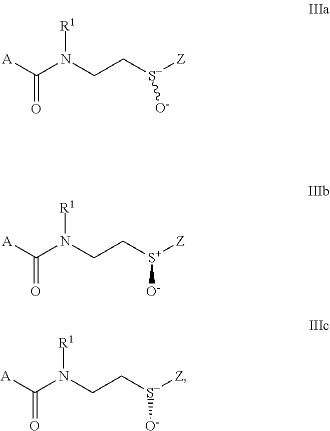

wherein each of A, R¹, and Z is as defined above and in the genera and subgenera herein.

In certain embodiments, the present invention encompasses compositions of matter comprising a therapeutically effective amount of one or more compounds of Formula IV:

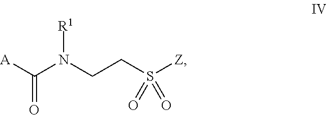

wherein each of A, R¹, and Z is as defined above and in the genera and subgenera herein.

In certain embodiments, the present invention encompasses compositions of matter comprising a therapeutically effective amount of one or more compounds of Formula IV:

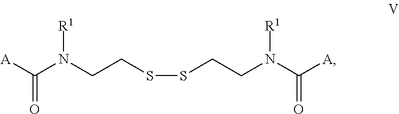

wherein each of A and R¹ is, independently, as defined above and in the genera and subgenera herein.

In certain embodiments, provided therapeutic compositions comprise one or more compounds of formulae I, II, III, IIIa, IIIb, IIIc, IV, or V, wherein the moiety -A is —H. In certain embodiments, the moiety -A comprises an optionally substituted C₁₋₃₉ aliphatic group.

In certain embodiments, the moiety -A comprises a straight chain C₃₋₂₄ aliphatic group. In certain embodiments, the moiety -A comprises a an unsaturated C₃₋₂₄ aliphatic group. In certain embodiments, the moiety -A comprises a C₁₋₃₉ alkyl group. In certain embodiments, the moiety -A comprises a C₁₋₃₂ alkyl group. In certain embodiments, the moiety -A comprises a straight chain C₃₋₁₈ alkyl group. In certain embodiments, the moiety -A comprises a straight chain C₃₋₁₂ alkyl group. In certain embodiments, the moiety -A comprises a C₁₋₈ alkyl group. In certain embodiments, the moiety -A comprises a C₁₋₆ alkyl group. In certain embodiments, the moiety -A comprises a C₁₋₄ alkyl group. In certain embodiments, the moiety -A is —CH₂CH₂CH₂CH₂CH₃. In certain embodiments, the moiety -A is —CH₂CH₂CH₂CH₃. In certain embodiments, the moiety -A is —CH₂CH(CH₃)₂. In certain embodiments, the moiety -A is —CH(CH₃)CH₂CH₃. In certain embodiments, the moiety -A is —CH₂CH₂CH₃. In certain embodiments, the moiety -A is —CH(CH₃)₂. In certain embodiments, the moiety -A is —CH₂CH₃. In certain embodiments, the moiety -A is —CH₃. In certain embodiments, -A is selected from the group consisting of: n-propyl, ethyl, methyl, and combinations of two or more of these.

In certain embodiments, provided therapeutic compositions comprise one or more compounds of formulae I, II, III, IIIa, IIIb, IIIc, IV, or V, wherein the moiety —R¹ is —H. In certain embodiments, provided therapeutic compositions comprise one or more compounds of formulae I, II, III, IIIa, IIIb, IIIc, IV, or V, wherein the moiety —R¹ comprises an optionally substituted C₁₋₄₀ aliphatic group. In certain embodiments, the moiety —R¹ comprises a straight chain C₃₋₂₄ aliphatic group. In certain embodiments, the moiety —R¹ comprises a an unsaturated C₃₋₂₄ aliphatic group. In certain embodiments, the moiety —R¹ comprises a C₁₋₄₀ alkyl group. In certain embodiments, the moiety —R¹ comprises a C₁₋₃₂ alkyl group. In certain embodiments, the moiety —R¹ comprises a straight chain C₃₋₁₈ alkyl group. In certain embodiments, the moiety —R¹ comprises a straight chain C₃₋₁₂ alkyl group. In certain embodiments, the moiety —R¹ comprises a C₁₋₈ alkyl group. In certain embodiments, the moiety —R¹ comprises a C₁₋₆ alkyl group. In certain embodiments, the moiety —R¹ comprises a C₁₋₄ alkyl group. In certain embodiments, the moiety —R¹ is —CH₂CH₂CH₂CH₂CH₃. In certain embodiments, the moiety —R¹ is —CH₂CH₂CH₂CH₃. In certain embodiments, the moiety —R¹ is —CH₂CH(CH₃)₂. In certain embodiments, the moiety —R¹ is —CH(CH₃)CH₂CH₃. In certain embodiments, the moiety —R¹ is —CH₂CH₂CH₃. In certain embodiments, the moiety —R¹ is —CH(CH₃)₂. In certain embodiments, the moiety —R¹ is —CH₂CH₃. In certain embodiments, the moiety —R¹ is —CH₃. In certain embodiments, —R¹ is selected from the group consisting of: —H, n-propyl, ethyl, methyl, and combinations of two or more of these.

In certain embodiments, provided therapeutic compositions comprise one or more compounds of formulae I, II, III, IIIa, IIIb, IIIc, or IV, wherein the moiety —Z comprises —H. In certain embodiments, provided therapeutic compositions comprise one or more compounds of formulae I, II, III, IIIa, IIIb, IIIc, or IV, wherein the moiety —Z is other than —H.

In certain embodiments, provided therapeutic compositions comprise one or more compounds of formulae I, II, III, IIIa, IIIb, IIIc, or IV, wherein the moiety —Z comprises —CH₃.

In certain embodiments, provided therapeutic compositions comprise one or more compounds of formulae I, II, III, IIIa, IIIb, IIIc, or IV, wherein the moiety —Z comprises an optionally substituted C₁₋₄₀ aliphatic group. In certain embodiments, the moiety —Z comprises a straight chain C₃₋₂₄ aliphatic group. In certain embodiments, the moiety —Z comprises a an unsaturated C₃₋₂₄ aliphatic group. In certain embodiments, the moiety —Z comprises a C₁₋₄₀ alkyl group. In certain embodiments, the moiety —Z comprises a C₁₋₃₂ alkyl group. In certain embodiments, the moiety —Z comprises a straight chain C₃₋₁₈ alkyl group. In certain embodiments, the moiety —Z comprises a straight chain C₃₋₁₂ alkyl group. In certain embodiments, the moiety —Z comprises a C₁₋₈ alkyl group. In certain embodiments, the moiety —Z comprises a C₁₋₆ alkyl group. In certain embodiments, the moiety -A comprises a C₁₋₄ alkyl group. In certain embodiments, the moiety —Z is —CH₂CH₂CH₂CH₂CH₃. In certain embodiments, the moiety —Z is —CH₂CH₂CH₂CH₃. In certain embodiments, the moiety —Z is —CH₂CH(CH₃)₂. In certain embodiments, the moiety —Z is —CH(CH₃)CH₂CH₃. In certain embodiments, the moiety —Z is —CH₂CH₂CH₃. In certain embodiments, the moiety —Z is —CH(CH₃)₂. In certain embodiments, the moiety —Z is —CH₂CH₃. In certain embodiments, —Z is selected from the group consisting of: n-propyl, ethyl, methyl, and combinations of two or more of these.

In certain embodiments, provided therapeutic compositions comprise one or more compounds of formulae I, II, III, IIIa, IIIb, IIIc, IV, or V, wherein the moiety —R¹ is —CH₃ and the moiety -A comprises an optionally substituted C₁₋₃₉ aliphatic group. In certain embodiments, the moiety —R¹ is —CH₃ and the moiety -A comprises a straight chain C₃₋₂₄ aliphatic group. In certain embodiments, the moiety —R¹ is —CH₃ and the moiety -A comprises a an unsaturated C₃₋₂₄ aliphatic group. In certain embodiments, the moiety —R¹ is —CH₃ and the moiety -A comprises a C₁₋₃₉ alkyl group. In certain embodiments, the moiety —R¹ is —CH₃ and the moiety -A comprises a C₁₋₃₂ alkyl group. In certain embodiments, the moiety —R¹ is —CH₃ and the moiety -A comprises a straight chain C₃₋₁₈ alkyl group. In certain embodiments, the moiety —R¹ is —CH₃ and the moiety -A comprises a straight chain C₃₋₁₂ alkyl group. In certain embodiments, the moiety —R¹ is —CH₃ and the moiety -A comprises a C₁₋₈ alkyl group. In certain embodiments, the moiety —R¹ is —CH₃ and the moiety -A comprises a C₁₋₆ alkyl group.

In certain embodiments, the moiety —R¹ is —CH₃ and the moiety -A comprises a C₁₋₄ alkyl group. In certain embodiments, the moiety —R¹ is —CH₃ and the moiety -A is —CH₂CH₂CH₂CH₂CH₃. In certain embodiments, the moiety —R¹ is —CH₃ and the moiety -A is —CH₂CH₂CH₂CH₃. In certain embodiments, the moiety —R¹ is —CH₃ and the moiety -A is —CH₂CH(CH₃)₂. In certain embodiments, the moiety —R¹ is —CH₃ and the moiety -A is —CH(CH₃)CH₂CH₃. In certain embodiments, the moiety —R¹ is —CH₃ and the moiety -A is —CH₂CH₂CH₃. In certain embodiments, the moiety —R¹ is —CH₃ and the moiety -A is —CH(CH₃)₂. In certain embodiments, the moiety —R¹ is —CH₃ and the moiety -A is —CH₂CH₃. In certain embodiments, the moiety —R¹ is —CH₃ and the moiety -A is —CH₃. In certain embodiments, -A is selected from the group consisting of: n-propyl, ethyl, methyl, and combinations of two or more of these.

In certain embodiments, provided therapeutic compositions comprise one or more compounds of formulae I, II, III, IIIa, IIIb, IIIc, IV, or V, wherein the moiety —R¹ is —CH₃ and the moiety A-C(O)— comprises an acyl group derived from an endogenous carboxylic acid. In certain embodiments, provided therapeutic compositions comprise one or more compounds of formulae I, II, III, IIIa, IIIb, IIIc, IV, or V, wherein the moiety —R¹ is —CH₃ and the moiety A-C(O)— comprises an acyl group derived from a microbiome-derived carboxylic acid. In certain embodiments, provided therapeutic compositions comprise one or more compounds of formulae I, II, III, IIIa, IIIb, IIIc, IV, or V, wherein the moiety —R¹ is —CH₃ and the moiety A-C(O)— comprises an acyl group derived from a bile acid.

In certain embodiments, provided therapeutic compositions comprise a compound selected from Table 1.

TABLE 1

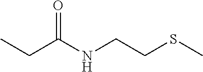

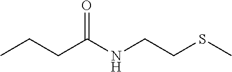

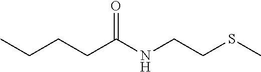

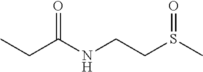

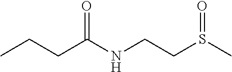

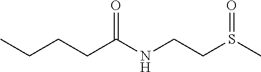

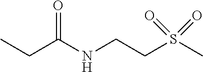

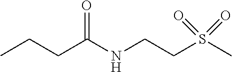

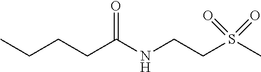

In certain embodiments, provided therapeutic compositions comprise a compound of formula:

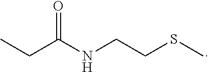

In certain embodiments, provided therapeutic compositions comprise a compound of formula:

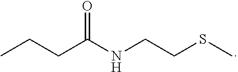

In certain embodiments, provided therapeutic compositions comprise a compound of formula:

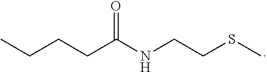

In certain embodiments, provided therapeutic compositions comprise a compound of formula:

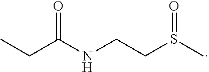

In certain embodiments, provided therapeutic compositions comprise a compound of formula:

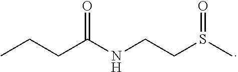

In certain embodiments, provided therapeutic compositions comprise a compound of formula:

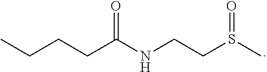

In certain embodiments, provided therapeutic compositions comprise a compound of formula:

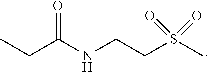

In certain embodiments, provided therapeutic compositions comprise a compound of formula:

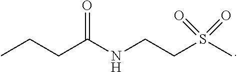

In certain embodiments, provided therapeutic compositions comprise a compound of formula:

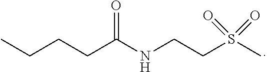

In certain embodiments, provided therapeutic compositions comprise cysteamides of bile acids, referred to herein as Bile Acid Cysteamides or BACs. The term “Bile Acid” in this context encompasses any carboxylic acid having a steroid carbon skeleton in its structure.

In certain embodiments, provided therapeutic compositions comprise S-alkyl cysteamides of bile acids. Such compounds are referred to herein as -alkyl bile acid cysteamides or SABACs.

In certain embodiments, provided therapeutic compositions comprise sulfoxides of bile acid cysteamides and their S-alkyl analogs. Such compounds are referred to herein as bile acid cysteamide oxides, and S-alkyl bile acid cysteamide oxides respectively, or BACOs, and SABACOs.

In certain embodiments, provided therapeutic compositions comprise sulfones of bile acid cysteamides and their S-alkyl analogs. Such compounds are referred to herein as bile acid cysteamide sulfones, and S-alkyl bile acid cysteamide sulfones respectively, or BACSOs, and SABACSOs.

In embodiments where provided therapeutic compositions comprise bile acid cysteamide derivatives or oxides or sulfones thereof having an S-alkyl substituent, the substituent may be any of those described above and in the genera and subgenera herein. In certain embodiments, provided therapeutic compositions comprise compounds conforming to Formula VI:

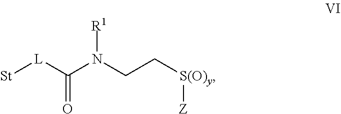

wherein:
   -L- comprises an optionally substituted, optionally unsaturated C₁₋₂₄ linker where any one or more carbon atoms may be optionally replaced with —NR^{y}—, —C(O)—, —O—, —S—, —S(O)—, —S(O)₂—, —C(═NR^{y})—, and —C(═S)—;
   -St comprises a moiety having a steroidal structure; and
   each of R¹, Z, and y is as defined above and in the genera and subgenera herein.

In certain embodiments, where provided therapeutic compositions comprise a compound of Formula VI, the moiety -L- comprises an optionally substituted linker moiety comprising a chain of 2 to 20 carbon atoms separating the moiety -St from the acyl linkage to the cysteamide. In certain embodiments, -L- comprises an optionally substituted linker moiety comprising a chain of 2 to 12 carbon atoms separating the moiety -St from the acyl linkage to the cysteamide. In certain embodiments, -L- comprises a chain of 3 to 7 carbon atoms separating the moiety -St from the acyl linkage.

In certain embodiments, the moiety -L- comprises

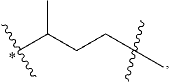 [SMILES: *CCC(*)C]

where * represents the site of attachment to the moiety -St.

In certain embodiments, the moiety -L- comprises

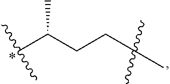 [SMILES: *CC[C@H](*)C]

where * represents the site of attachment to the moiety -St.

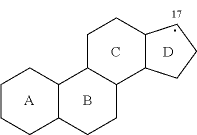

In certain embodiments, the moiety -St comprises a steroid moiety linked to -L-through ring-D.

In certain embodiments, the moiety -St comprises a steroid moiety linked to -L-through carbon 17.

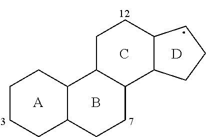

In certain embodiments, the steroid ring of the moiety -St is substituted with 1 or more hydroxyl groups. In certain embodiments, the steroid ring of the moiety -St is substituted with 3 hydroxyl groups. In certain embodiments, the steroid ring of the moiety -St is substituted with 2 hydroxyl groups. In certain embodiments, the steroid ring of the moiety -St is substituted 1 hydroxyl group. In certain embodiments, at least one hydroxyl substituent is at carbon 3, 7, or 12 of the steroid ring.

In certain embodiments, the moiety -St-L- in Formula VI is selected from the group consisting of:

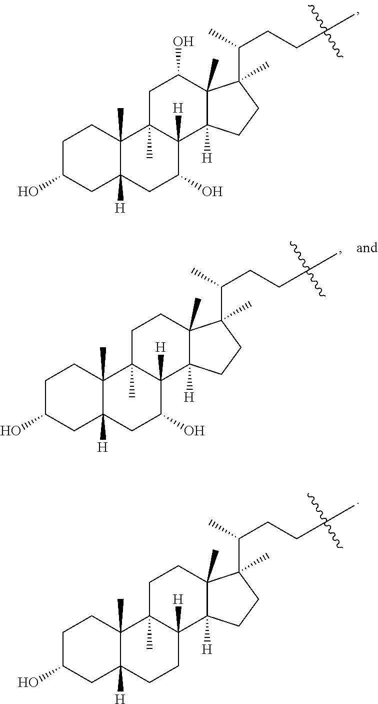

In certain embodiments, the moiety Z in Formula VI is methyl. In certain embodiments, R¹ in Formula V is —H. In certain embodiments, Z¹ in Formula V is methyl, the moiety R¹ is —H, and the moiety -St-L- is selected from the group consisting of:

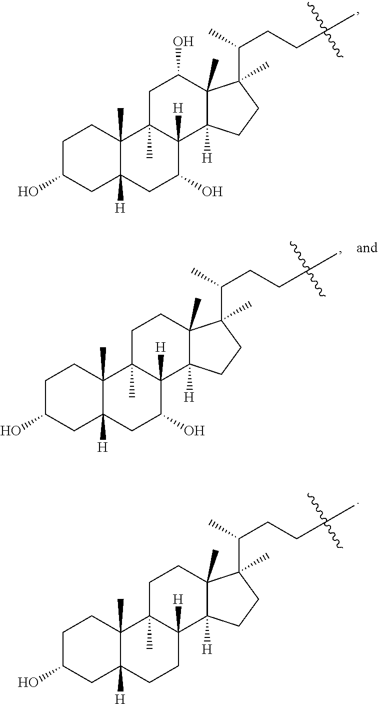

In certain embodiments, provided therapeutic compositions comprise a compound selected from Table 2:

TABLE 2

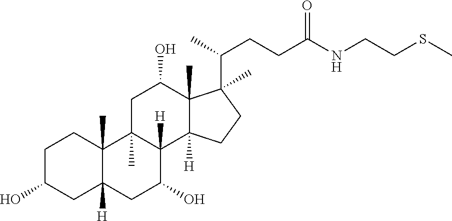

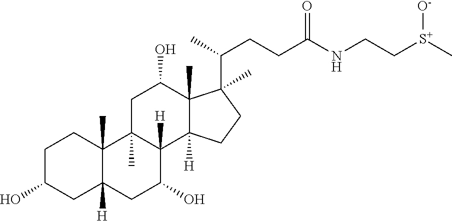

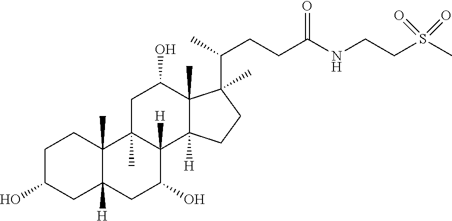

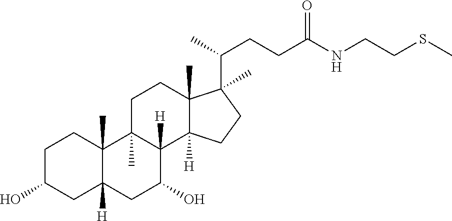

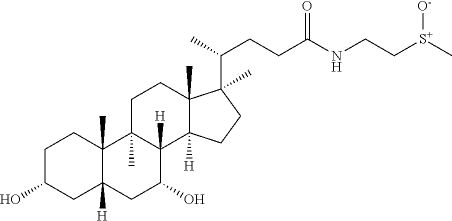

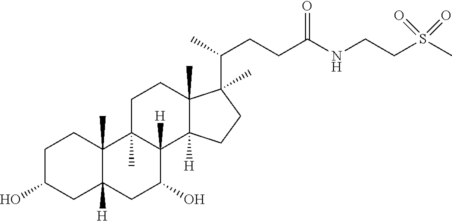

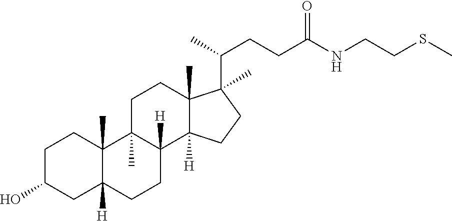

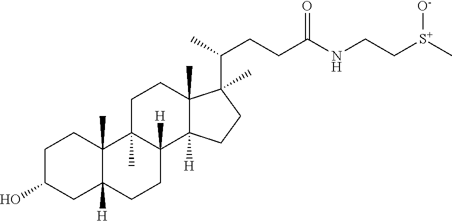

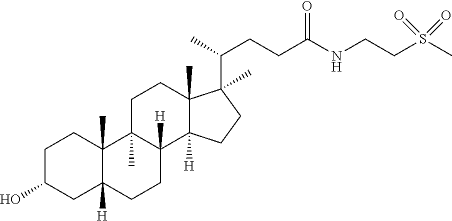

In certain embodiments, provided therapeutic compositions comprise a compound of formula:

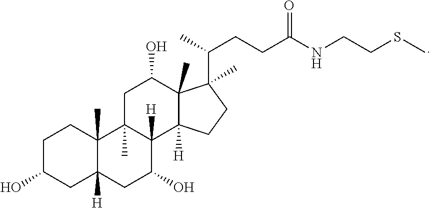

In certain embodiments, provided therapeutic compositions comprise a compound of formula:

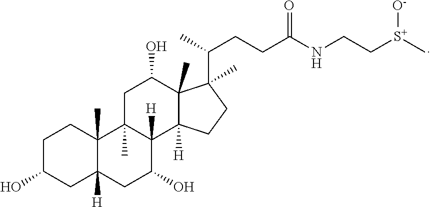

In certain embodiments, provided therapeutic compositions comprise a compound of formula:

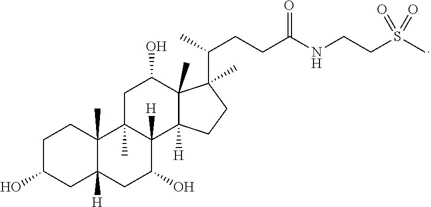

In certain embodiments, the present invention provides therapeutic compositions comprising a mixture of any of the cysteamides described above in combination with an S-oxidized congener of the same compound. In certain embodiments, such compositions comprise a mixture of an un-oxidized cysteamide and a sulfoxide of that cysteamide. In certain embodiments, such compositions comprise a mixture of an un-oxidized cysteamide and a sulfone of that cysteamide. In certain embodiments, such compositions comprise a mixture of an un-oxidized cysteamide, in combination with the sulfoxide and the sulfone of that cysteamide.

In another aspect, the present invention provides pharmaceutical compositions containing cysteamides (e.g. any one or more compounds provided herein and/or conforming to Formulae I, II, III, IIIa, IIIb, IIIc, IV, V, or VI described above). In certain embodiments, the invention encompasses a pharmaceutical composition or a single unit dosage form of any of the cysteamide compounds described above. In certain embodiments, pharmaceutical compositions and single unit dosage forms of the invention comprise a prophylactically or therapeutically effective amount of one or more of the cysteamides describe above, or their pro-drugs, and typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmacopeia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This invention further encompasses anhydrous pharmaceutical compositions and dosage forms comprising active ingredients (e.g. any of the cysteamides described above), since water can facilitate the degradation of some compounds. For example, the addition of water (e.g., 5%) is widely accepted in the pharmaceutical arts as a means of simulating long-term storage to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise any one or more cysteamides and one or more compounds that reduce the rate by which an active ingredient will decompose. Such compounds, herein referred to as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In certain embodiments, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In certain embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocane to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the invention comprise a compound of the invention, or a pharmaceutically acceptable salt, solvate or hydrate thereof lie within the range of from about 1 mg to about 10,000 mg per day, given as a single once-a-day dose in the morning but preferably as divided doses throughout the day taken with food.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103.TM. and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal, Topical & Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Dietary Supplement and Functional Food Forms

In certain embodiments, compounds of the present invention can be added to food or compounded with food additives and provided as a part of a subject's diet. In certain embodiments, a cysteamide derivative of the present invention (e.g. any one or more compounds provided herein and/or conforming to Formulae I, II, III, IIIa, IIIb, IIIc, IV, V, or VI described above) is added to a prepared or packaged food item or included in a vitamin or dietary supplement. Representative examples include adding the cysteamides to formula, milk or other dairy products, powdered milk, protein powder, beverages, health drinks, teas, juices and the like. In certain embodiments, the cysteamides are added to multivitamins or to other dietary supplements and similar products.

### Chemical Compositions of Matter

In another aspect, the present invention encompasses novel compositions of matter including compositions of novel molecules. While some of the cysteamides described above are naturally occurring molecules that have been detected in the bodies of mice or other animals or have been shown to be produced by organisms in the microbiomes of mice or other creatures, pure samples of these molecules and, in particular, bulk samples of pure cysteamides free from other biological materials are not found in nature. Additionally, many of the Cysteamides and related compounds described above have not been detected in nature, even with the aid of highly sensitive and selective analytical techniques such as HPLC-coupled high resolution mass spectroscopy. As such, many of the compounds described above constitute novel non-natural compositions of matter.

In certain embodiments, the present invention provides pure samples of any of the cysteamides, S-alkyl cysteamides, cysteamide S-oxides, S-alkyl cysteamide S-oxides, cysteamide sulfones, and S-alkyl cysteamide sulfones described above and in the genera and subgenera herein. In certain embodiments, the present invention provides samples comprising bulk quantities of such molecules in substantially pure form. In certain embodiments, the present invention provides samples comprising at least 100 mg, at least 1 g, at least 10 g, at least 50 g, at least 200 g, at least 500 g, or at least 1 kg of such molecules in substantially pure form.

In certain embodiments, the present invention provides novel compositions comprising one or more compounds depicted in Table 1. In certain embodiments, the present invention provides novel compositions comprising one or more compounds depicted in Table 2. In certain embodiments, the present invention provides novel compositions comprising mixtures of between two and ten different cysteamides.

In certain embodiments, the present invention provides novel compositions comprising a mixture of an un-oxidized cysteamide as described herein and an S-oxidized congener of the same cysteamide. In certain embodiments, such compositions comprise a mixture of an un-oxidized cysteamide and a sulfoxide of that cysteamide. In certain embodiments, such compositions comprise a mixture of an un-oxidized cysteamide and a sulfone of that cysteamide. In certain embodiments, such compositions comprise a mixture of an un-oxidized cysteamide, in combination with the sulfoxide and the sulfone of that cysteamide. In certain embodiments, the present invention provides compositions comprising a mixture of sulfoxide of a cysteamide as described herein and a sulfone of that cysteamide.

### Therapeutic Methods

In another aspect, the present invention encompasses methods of improving the health of an animal or of treating or ameliorating a health disorder in an animal by administering to the animal an effective amount of any one or more of the therapeutic compositions described above (e.g. a composition comprising any one or more compounds provided herein and/or conforming to Formulae I, II, III, IIIa, IIIb, IIIc, IV and V described above). In certain embodiments, the method comprises administering such a composition to a mammal. In certain embodiments, the method comprises administering such a composition to a human.

In certain embodiments, provided therapeutic methods comprise administration of cysteamides of short-chain fatty acids (SCFAs) (e.g. administration of any of the compounds of formulae I, II, III, IIIa, IIIb, IIIc, IV and V where -A comprises an SCFA moiety). SFCAs are known to be bacterial fermentation products, which are chemically composed of a carboxylic acid moiety and a small hydrocarbon chain. Among them, acetic, propionic and butyric acids are the most studied, presenting, respectively, two, three and four carbons in their chemical structure. These metabolites are found in high concentrations in the intestinal tract, and are taken up by intestinal epithelial cells (IECs). The biological roles of SCFAs in mammals are varied and therefore suggest a number of potential therapeutic areas where the provided therapeutic compositions have utility.

The SCFAs are known to modify several cellular processes including gene expression, chemotaxis, differentiation, proliferation and apoptosis. For example, SCFAs are partially used as a source of ATP by IEC cells. In addition, these molecules act as a link between the microbiota and the immune system. Provided compositions can therefore modulate different aspects of IECs and modulate leukocyte development, survival and function (for example, through activation of G protein coupled receptors (e.g. FFAR2, FFAR3, GPR109a and Olfr78) and by modulation of the activity of enzymes and transcription factors (including, for example the histone acetyltransferase and deacetylase, and the recently described stabilization of the hypoxia-inducible factor (HIF) are implicated in their effects (see, for example: Donohoe D R, Collins L B, Wali A, Bigler R, Sun W, Bultman S J. The warburg effect dictates the mechanism of butyrate-mediated histone acetylation and cell proliferation. Mol Cell 2012; 48: 612-626; inolo M A, Rodrigues H G, Nachbar R T, Curi R. Regulation of inflammation by short chain Fatty acids. Nutrients 2011; 3: 858-876; Vinolo M A, Hirabara S M, Curi R. G-protein-coupled receptors as fat sensors. Curr Opin Clin Nutr Metab Care 2012; 15: 112-116; and Kelly C J, Zheng L, Campbell E L, Saeedi B, Scholz C C, Bayless A J et al. Crosstalk between microbiota-derived short-chain fatty acids and intestinal epithelial HIF augments tissue barrier function. Cell Host Microbe 2015; 17: 662-671, each of which is incorporated herein by reference). SCFAs activate at least four different GPCRs: the free fatty acid receptors (FFAR)-2 and -3, which are also known as GPR43 and GPR41, respectively, the niacin/butyrate receptor GPR109a (also known as HCA2) and the olfatory receptor (Olfr)-78 (see, for example. Pluznick J. A novel SCFA receptor, the microbiota, and blood pressure regulation. Gut Microbes 2014; 5: 202-207; and Thangaraju M, Cresci G A, Liu K, Ananth S, Gnanaprakasam J P, Browning D D et al. GPR109A is a G-protein-coupled receptor for the bacterial fermentation product butyrate and functions as a tumor suppressor in colon. Cancer Res 2009; 69: 2826-2832, each of which is incorporated herein by reference). These receptors show distinct patterns of expression and they have been partially associated with the effects of the SCFAs on leukocytes and intestinal epithelial cells (IECs). More details on the molecular mechanisms and on the effects of SCFAs in other tissues can be found in different reviews at the literature, see for example: den Besten G, van Eunen K, Groen A K, Venema K, Reijngoud D J, Bakker B M. The role of short-chain fatty acids in the interplay between diet, gut microbiota, and host energy metabolism. J Lipid Res 2013; 54: 2325-2340 which is also incorporated herein by reference)).

Considering this, administration of the compositions provided herein (and in particular those containing cysteamides of SCFAs) provides valuable new methods to maintain intestinal homeostasis and to treat disorders arising from imbalances in this system including, but not limited to: periodontal disease, bacterial vaginosis, inflammatory bowel disease, rheumatoid arthritis, obesity, asthma/allergy, psoriasis, multiple sclerosis and cancer.

The SCFAs are known to modify several cellular processes including, but not limited to innate and adaptive lymphocyte development and function. Some SCFAs and their receptors can influence innate lymphoid cell functions that can regulate innate and adaptive immune cell responses and acute and chronic inflammatory processes associated with inflammatory bowel disease, rheumatoid arthritis, obesity, psoriasis, asthma/allergy, MS and cancer. In particular, group 3 innate lymphoid cell activation, recruitment and functional potential can be influenced by ligands of GPR43, GPR183 and other family members. In addition, multiple functions of regulatory T cells that limit inflammation can be regulated by the microbiota, their metabolites and signaling via GPCRs.

The inventors have shown that administration of SCFA-MCY conjugates reduce levels of resident regulatory T cells (Tregs) in different tissues, including the brain. Tregs are key regulators of immune responses and have been implicated in the control of immune cell activation, proliferation, and effector function. These activity involve a wide range of signaling pathways and mechanisms, e.g., inhibitory cytokines, cytotoxic molecules, modulators of cAMP and cytokine competition. Promotion or suppression of different aspects of Treg functions can be an important component of therapeutic strategies (ncbi.nlm.nih.gov/pmc/articles/PMC2434375/).

Thus, compositions provided herein (and in particular those containing cysteamides of SCFAs) can be used to inhibit, reduce, and/or suppress Tregs. In certain embodiments, the methods are in vitro. In certain embodiments, the methods are in vivo. In certain embodiments, the methods comprise administering a composition provided herein (and in particular those containing cysteamides of SCFAs) to an animal in need thereof (such as those described hereinbelow).

While Tregs have central roles in the maintenance of self-tolerance, as they protect hosts from developing autoimmune diseases and allergies, they hinder immune surveillance against cancer in healthy individuals and prevent the development of effective antitumor immunity in tumor-bearing patients. Treg have been shown to infiltrate into the tumor microenvironment (TME) in multiple murine and human tumors and impede anti-tumor immunity via diverse mechanisms including suppression of antigen-presenting cells, production of immune inhibitory cytokines, and T-cell exhaustion (PMID: 30705439, 30936494). Therefore, interventions to deplete Treg cells offer new perspectives to increase anti-tumor immune responses, addressing an urgent need in the cancer immunotherapy field (pubmed.ncbi.nlm.nih.gov/31102428/). Specifically, blocking and/or depleting Tregs has emerged as a viable strategy to enhance antitumor immunity. Based on these insights, administration of the compositions provided herein (and in particular those containing cysteamides of SCFAs) provide valuable new methods for the development of Treg cell-targeted cancer immunotherapies. For the treatment of cancer and other malignancies, the compositions provided herein (and in particular those containing cysteamides of SCFAs) can be administered systemically or targeted to specific sites, e.g. specific tissues or tumors and metastases. Specific targeting of cysteamide derivatives to the tumor can be achieved by conjugating the cysteamide to a tumor-targeting antibody, e.g., using a photocleavable linkage through the amide nitrogen (onlinelibrary.wiley.com/doi/full/10.1002/eji.202048992). For example, adapting known approaches for tumor targeting (Sato, et al., Sci. Transl. Med. (2016) 8:352), antibody targeting a tumor-specific antigen is conjugated to a photoactivatable dye that is sensitive to near infrared (NIR) light. NIR irradiation is then administered locally to the tumor, thus, enabling intratumoral release of the cysteamide.

In addition, the suppression of Treg cells by compositions provided herein (and in particular those containing cysteamides of SCFAs) can improve the efficacy of vaccinations. Vaccination is known to induce expansion of Treg populations, e.g., at the site of vaccine injection (pubmed.ncbi.nlm.nih.gov/29467445/). Further, it has been demonstrated that suppression of such Treg expansion upon vaccination can increase efficacy of vaccination, e.g., in the case of influenza viral clearance. Thus, compositions provided herein (and in particular those containing cysteamides of SCFAs) can offer medical benefits as vaccine additives or coadminstered agent, for example by inhibiting Treg development and enhancing T cell immunity. This method is particularly promising for development of vaccines against parasites including malaria, filaria, and helminths for which no vaccines are available to date (PMID 16766171).

In addition, the suppression of Treg cells by compositions provided herein (and in particular those containing cysteamides of SCFAs) provides valuable new methods for the treatment of immunosuppressive disease. For example, while Tregs are considered both beneficial and detrimental during acute HIV infection (ncbi.nlm.nih.gov/pmc/articles/PMC5908895/#:˜:text=Treg %20Suppression %20in %20HIV %20Infection,118%2C%20149%E2%80%93152), expansion of Tregs driven by human immunodeficiency virus (HIV) suppress HIV-specific CD4 T-cell responses in HIV-infected patients (sciencedirect.com/science/article/pii/S0006497120558850). Moreover, Tregs are promoting the generation of the latent HIV/SIV reservoir, the seeding of which starts as early as 3 days post-infection (pubmed.ncbi.nlm.nih.gov/25042999/), which represents the ultimate obstacle for HIV cure research.

In addition, the suppression of Treg cells by compositions provided herein (and in particular those containing cysteamides of SCFAs) provides valuable new methods for the treatment of chronic infectious disease. For example, Tregs are considered detrimental for the control of tuberculosis infection since the immunosuppressive effects of Tregs at the early stage of infection can promote dissemination of bacteria in the lung tissue (PMID 31572365).

In addition, the suppression of Treg cells by compositions provided herein (and in particular those containing cysteamides of SCFAs) provides valuable new methods for the treatment of idiopathic pulmonary fibrosis (IPF). For example, Tregs have been shown to play a profibrotic role by promoting collagen accumulation and histological determinants of tissue remodeling in the fibrotic mammalian lung therefore exacerbating IPF progression (PMID 27344432). Given the paucity of effective treatments for IPF and there multitude of poorly tolerated side effects, the compositions provided herein provide a novel therapeutic avenue.

In addition, the suppression of Treg cells by compositions provided herein (and in particular those containing cysteamides of SCFAs) provides valuable new methods for the modulation of aging-induced immune dysregulation. Regulatory T cells are known to accumulate over the lifetime of humans and may play important roles in increased susceptibility to cancer, infections, reduced vaccine-mediated immunity, and increased tissue degeneration. Therefore, the compositions provided herein provide novel approaches for modulating declining immune function and its consequences in aging humans (PMID: 33584708).

In certain embodiments, provided therapeutic methods comprise administration of cysteamides of bile acids (BAs). BAs and their taurine-conjugated derivatives play diverse roles in human metabolism. As ligands of the nuclear receptor FXR, bile acids regulate cholesterol homeostasis, liver regeneration, and inflammation as well as lipid metabolism and glucose metabolism, and furthermore contribute to inter-organ communication, In addition, regulation of FXR activity plays a role in cancer. For example, regulation of FXR activity by bile acids has been linked to differential outcomes in colorectal cancer. Moreover, FXR expression is positively correlated with tumor size and the proliferative rate of e.g. breast cancer, and FXR expression is significantly increased in some types of lung cancer (Int J Mol Sci. 2018 July; 19(7): 2069). FXR has been shown to be associated with a higher tumor grade, greater tumor size and lymph node metastasis in esophageal adenocarcinomas. In addition, FXR is hypothesized to serve functional roles in the nervous system.

Cysteamides of BAs function as potent antagonists of FXR. FXR antagonists have been shown to protect against liver injury in cholestasis, lower cholesterol, suppresses gluconeogenesis in mouse primary hepatocytes, and improves glucose homeostasis in HFD/STZ-induced T2DM mice. Moreover FXR antagonists have anti-cancer activities that suggest use in both prevention and treatment of cancer. For example, a natural product-derived FXR antagonist inhibited proliferation of cancer cell lines, and induced cell apoptosis in esophageal cancer, pancreatic cancer, and head and neck cancer, and other cancers.

In certain embodiments, provided therapeutic methods comprise administration of cysteamides of bile acids (BAs). BAs and their taurine-conjugated derivatives play diverse roles in human metabolism. As ligands of the nuclear receptor FXR, bile acids regulate cholesterol homeostasis, liver regeneration, and inflammation as well as lipid metabolism and glucose metabolism, and furthermore contribute to inter-organ communication, In addition, regulation of FXR activity plays a role in cancer. For example, regulation of FXR activity by bile acids has been linked to differential outcomes in colorectal cancer. Moreover, FXR expression is positively correlated with tumor size and the proliferative rate of e.g. breast cancer, and FXR expression is significantly increased in some types of lung cancer (Int J Mol Sci. 2018 July; 19(7): 2069). FXR has been shown to be associated with a higher tumor grade, greater tumor size and lymph node metastasis in esophageal adenocarcinomas. In addition, FXR is hypothesized to serve functional roles in the nervous system.

Cysteamides of BAs function as potent antagonists of FXR. FXR antagonists have been shown to protect against liver injury in cholestasis, lower cholesterol, suppresses gluconeogenesis in mouse primary hepatocytes, and improves glucose homeostasis in HFD/STZ-induced T2DM mice. Moreover FXR antagonists have anti-cancer activities that suggest use in both prevention and treatment of cancer. For example, a natural product-derived FXR antagonist inhibited proliferation of cancer cell lines, and induced cell apoptosis in esophageal cancer, pancreatic cancer, and head and neck cancer, and other cancers.

### Cysteamides as Therapies to Modulate the Immune System

In certain embodiments, provided compositions (e.g. one or more compounds provided herein and/or conforming to Formulae I, II, III, IIIa, IIIb, IIIc, IV, V, and VI described above), or compositions comprising one or more of these compounds, have utility as therapeutics to modulate the immune system of an animal.

In certain embodiments, the present invention comprises a method of reducing or ameliorating chronic inflammatory diseases ranging from the skin (psoriasis and atopic dermatitis), oral cavity (periodontal disease), airways (asthma/allergy), gastrointestinal tract (food allergy, IBD, IBS, celiac disease, cancer), obesity and cancer. The impact of specific compounds on the type of inflammation but be context dependent and dictated by the specific tissue and nature of the inflammatory lesions. Immediate examples include manipulation of T helper subsets (Th1, Th2, Th17, Treg), innate lymphoid cells (ILC1, 2, 3) and myeloid and granulocyte lineages.

### Cysteamides as Modulators of Histone Acetylation

In certain embodiments, provided cysteamides (e.g. one or more compounds provided herein and/or conforming to Formulae I, II, III, IIIa, IIIb, IIIc, IV, V, and VI described above), or compositions comprising one or more of these compounds, have utility as therapeutics that act by modulating histone acetylation or deacetylation.

Butyric acid cysteamide derivatives could modulate histone acetylation (more generally, acylation) levels, either by inhibiting acyl transferases (which attach the acyl groups to the lysines of the histones) or by modulating (likely inhibiting, as an antagonist) sirtuins and other histone deactylases (HDACs). This makes therapeutic agents and methods based on these molecules highly relevant to the treatment of cancer.

### SCFA Cysteamides to Treat or Prevent Cancer and Proliferative Disorders

Taking into account the fact that carboxylic acids and SCFAs in particular are important energetic substrates for epithelial cells and the fact that they are regulators of their proliferation, it is believed treatment with cysteamides can modulate cellular proliferation. For example, cysteamides of SCFAs, (particularly of butyrate), may exert different effects on the growth of normal and tumoral colonocytes. For example, butyrate inhibits the growth of cancerous colonic cells, but not of normal colonocytes and, depending on the concentration, it actually increases the proliferation of this latter cell type. Treatment with cysteamides of SCFA's therefore provides a useful method of modulating proliferation of colonic cells and therefore of treating diseases such as colon cancer.

Similar patterns features are known to be present in other cancerous cells and therefore treatment with the provided cysteamide compositions (e.g. compositions containing one or more compounds provided herein and/or conforming to Formulae I, II, III, IIIa, IIIb, IIIc, IV, V, and VI described above) provide a useful therapy for treating or preventing cancer.

Based on the established role of FXR activation in a wide range of cancers, e.g., esophageal cancer, pancreatic cancer, lung cancer, and head and neck cancer, BA cysteamides as FXR antagonists have utility as therapeutics for the prevention and treatment of diverse cancers and related proliferative disorders. Thus, in certain embodiments, the present invention comprises a method of treating or preventing cancer and other proliferative disorders by treatment via oral or intravenous administration of a composition containing BA cysteamides, optionally in combination with other cancer treatments, e.g. surgery, radiation treatment, chemotherapy, hormonal treatment, and/or immunotherapy.

### Bile Acid Cysteamides as Therapies for Cancer

Based on the established role of FXR activation in a wide range of cancers, e.g., esophageal cancer, pancreatic cancer, lung cancer, and head and neck cancer, BA cysteamides as FXR antagonists have utility as therapeutics for the prevention and treatment of diverse cancers and related proliferative disorders. Thus, in certain embodiments, the present invention comprises a method of treating or preventing cancer and other proliferative disorders by treatment via oral or intravenous administration of a composition containing BA cysteamides, optionally in combination with other cancer treatments, e.g. surgery, radiation treatment, chemotherapy, hormonal treatment, and/or immunotherapy.

### Bile Acid Cysteamides as Therapies to Regulate Metabolic Disorders

The ligand-regulated nuclear receptor FXR plays a central role in lipid and glucose metabolism as well as cholesterol homeostasis, and BA cysteamides are potent FXR antagonists. High cholesterol levels underlie a wide range of human diseases, including blood clots, cardiovascular disease, peripheral arterial disease, type 2 diabetes, and stroke. FXR antagonists have been shown to reduce liver cholesterol (PMID 11988537) and therefore have immense therapeutic potential.

In certain embodiments, the present invention comprises a method of lowering cholesterol levels, including low-density lipoprotein and triglyceride levels. In certain embodiments, the present invention comprises a method for treating or preventing hyperlipidemia, blood clots, cardiovascular disease, fatty liver disease, peripheral arterial disease, and stroke. In addition, the present invention in certain embodiments comprises a method for treating or preventing hyperglycemia and/or type 2 diabetes.

### Methods of Enhancing the Ability of Therapeutics to Cross the Blood Brain Barrier.

As noted above, there is evidence that cysteamide derivatization of molecules such as SCFAs and bile acids can enhance the ability of those molecule to cross the blood brain barrier. The inventors believe this phenomenon can be generalized and that cysteamide derivatization may provide a valuable strategy for enhancing the ability of molecules to cross the blood brain barrier. As such, in one aspect the present invention provides methods of enhancing the ability of a therapeutic molecule to cross the blood brain barrier, the method comprising forming a cysteamide derivative of the therapeutic molecule. In certain embodiments, the method comprises derivatizing therapeutic agents that are neurologically active (e.g. psychoactive drugs, antidepressants and the like). In certain embodiments, the method comprises forming a cysteamide derivative of a neuropharmacological agent to increase its ability to cross the blood brain barrier. For molecules that do not contain a free carboxylate group that can be directly derivatized as a cysteamide, the derivatization may be accomplished by utilizing a bifunctional linker, for example, a hydroxyl group or an amide might be derivatized with a dicarboxylic acid derivative to form an ester or amide respectively with the other carboxylic group then being functionalized as a cysteamide as described herein. In a related aspect, the present invention provides methods of treating a patient in need of neuropharmacological treatment with the cysteamide derivative of a relevant neuropharmacological agent. In certain embodiments, such methods are characterized in that the bioavailability of the neuropharmacological agent in the brain is higher when the subject is treated with the cysteamide derivative of the agent, compared to treatment with the underivatized neuropharmacological agent.

### Cysteamides and Related Molecules as Treatments to Improve Mood or Mental State, and to Treat Neurological Disorders

As noted above, cysteamide derivatives have enhanced ability to cross the blood brain barrier and thus may have utility as neuropharmacological agents. Therefore, in certain embodiments, provided cysteamides (e.g. one or more compounds conforming to Formulae I, II, III, IIIa, IIIb, IIIc, IV, V, and VI described above), or compositions comprising one or more of these compounds have utility as therapeutics to cure or ameliorate neurological diseases.

In certain embodiments, the present invention comprises a method of reducing or ameliorating symptoms from anxiety, depression, bipolar disorder, attention deficit disorder, insomnia, schizophrenia, or other similar conditions and disorders, comprising the step of administering an effective amount of one of the therapeutic compositions described above to a subject suffering from one or more of these conditions.

In certain embodiments, the present invention comprises a method of improving cognitive development or function by administering an effective amount of one of the therapeutic compositions described above.

### EXAMPLES

The following Examples are useful to confirm aspects of the disclosure described above and to exemplify certain embodiments of the disclosure.

### Methods

Metabolite extraction from mouse brain. Intact mouse brain was frozen and stored at −80° C. before processing. Frozen brain was crushed and grinded with a pre-chilled mortar and pestle. Dry ice was added to mortar and pestle throughout the homogenization process to prevent thawing. Resulting powdered brain samples were sonicated for 1 min with 5 mL methanol in 20 mL glass scintillation vials, using 10 μL solvent per mg, followed by another 10 min of vigorous stirring. Extracts were pelleted at 5,000 g for 5 min, and supernatants were transferred to another 20 mL glass vials. Remaining pellets were further extracted with another 10 min of vigorous stirring in 5 mL ethanol. The supernatants were combined and then dried in a SpeedVac (ThermoFisher Scientific) vacuum concentrator. Dried materials were resuspended in 300 μL of methanol. Samples were pelleted at 5,000 g for 5 min and clarified extracts were transferred to fresh HPLC vials and stored at −20° C. until analysis.

Metabolite extraction from mouse serum samples. 800 μL of methanol were added to 200 μL of serum in 1.7 mL Eppendorf tubes. The tubes were sonicated for 1 min followed by another 10 min of vigorous stirring. Extracts were pelleted at 5,000 g for 5 min, and supernatants were transferred to 2 mL HPLC vials. Remaining pellets were further extracted with another 10 min of vigorous stirring in 0.5 mL ethanol. Extracts were pelleted at 5,000 g for 5 min, and the combined supernatants were then dried in a SpeedVac (ThermoFisher Scientific) vacuum concentrator. Samples were then resuspended in 150 L of methanol. Samples were pelleted at 5,000 g for 5 min and clarified extracts were transferred to fresh HPLC vials and stored at −20° C. until analysis.

Analytical methods and equipment overview. (a) Mass spectrometry: High resolution LC-MS was performed on a Thermo Fischer Scientific Vanquish UHPLC system coupled with a Thermo Q-Exactive HF hybrid quadrupole-orbitrap high-resolution mass spectrometer equipped with a HESI ion source. Metabolites were separated using a water-acetonitrile gradient on a Thermo Scientific Hypersil GOLD C18 column (150 mm×2.1 mm, particle size 1.8 m) maintained at 40° C.; solvent A: 0.1% formic acid in water; solvent B: 0.1% formic acid in acetonitrile. The A/B gradient started at 1% B for 3 min after injection and increased linearly to 100% B at 20 min, then 100% B for 5 min, and down to 1% B for 3 min using a flow rate of 0.5 mL/min was applied for a C18 column. Mass spectrometer parameters: spray voltage 3.5 kV, capillary temperature 380° C., prober heater temperature 400° C.; 60 sheath flow rate, 20 auxiliary flow rate, and one spare gas; S-lens RF level 50, resolution 240,000, AGC target 3×10⁶. The instrument was calibrated weekly with positive and negative ion calibration solutions (ThermoFisher). Each sample was analyzed in negative and positive ionization modes using a m z range of 100 to 800. (b) NMR spectroscopy: NMR spectroscopy was performed on a Varian INOVA 600 MHz NMR spectrometer (600 MHz ¹H reference frequency, 151 MHz for ¹³C) equipped with an HCN indirect-detection probe. Non-gradient phase-cycled dqfCOSY spectra were acquired using the following parameters: 0.6 s acquisition time; 400-600 complex increments; 8, 16 or 32 scans per increment. HSQC and HMBC spectra were acquired with these parameters: 0.25 s acquisition time, 200-500 increments, 8-64 scans per increment. ¹H, ¹³C-HMBC spectra were optimized for J_{H,C}=6 Hz. HSQC spectra were acquired with or without decoupling. NMR spectra were processed and baseline corrected using MestreLabs MNOVA software packages.

Feature detection and characterization. LC-MS RAW files for all brain and serum samples were converted to mzXML format (centroid mode) using MSconvert (ProteoWizard), followed by analysis using the XCMS analysis feature in Metaboseek (metaboseek.com) based on the centWave XCMS algorithm to extract features^{1,2}. Peak detection values were set as: 4 ppm, 3 to 20 peakwidth, 3 snthresh, 3 and 100 prefilter, FALSE fitgauss, 1 integrate, TRUE firstBaselineCheck, 0 noise, wMean mzCenterFun, −0.005 mzdiff. XCMS feature grouping values were set as: 0.2 minfrac, 2 bw, 0.002 mzwid, 500 max, 1 minsamp, FALSE usegroup. Metaboseek peak filling values set as: 5 ppm_m, 5 rtw, TRUE rtrange. Resulting tables of all detected features were then processed with the Metaboseek data explorer. To select differential features, a filter was applied retaining entries with peak area ratios smaller than 1/3 (down in GF mice) or larger than 3 (up in GF mice), with a retention time window of 1 to 20 min, and >0.97 Peak Quality as calculated by METABOseek³. The resulting list was manually curated to remove false positive entries, i.e., features that upon manual inspection of raw data were not differential. For verified differential features, elution profiles, isotope patterns, and MS1 spectra were examined to find molecular ions and remove adducts, fragments, and isotope peaks. Remaining masses were put on the inclusion list for MS/MS (ddMS2) characterization. Positive and negative ionization mode data were processed separately. To acquire MS2 spectra, a top-10 data dependent MS2 method was run on a Thermo Q-exactive-HF mass spectrometer with MS1 resolution 60,000, AGC target 1×10⁶, maximum IT (injection time) 50 ms, MS2 resolution 45,000, AGC target 5×10⁵, maximum IT 80 ms, isolation window 1.0 m z, stepped NCE (normalized collision energy) 10 and 30 for positive and negative ionization mode, dynamic exclusion 3 s.

MS2-based molecular networking. A MS2 molecular network was created using Metaboseek version 0.9.7 and visualized in Cytoscape⁴. Features were matched with their respective MS2 scan within an m z window of 5 ppm and a retention time window of 15 s, using the MS2 scans function. To construct the molecular network, tolerance of the fragment peaks was set to m/z of 0.002 or 5 ppm, minimum number of peaks was set to 3, with a 2% noise level. Once the network was constructed, a cosine value of 0.7 was used, and the number of possible connections was constrained to 6 for both negative ion mode and positive ion mode.

General synthetic procedures. Unless noted otherwise, all chemicals and reagents were purchased from Sigma-Aldrich. Solutions and solvents sensitive to moisture and oxygen were transferred via standard syringe and cannula techniques. Acetic acid (AcOH), acetonitrile (ACN), dichloromethane (DCM), and methanol (MeOH) used for chromatography and as a reagent or solvent were purchased from Fisher Scientific. Flash chromatography was performed using Teledyne Isco CombiFlash systems and Teledyne Isco RediSep Rf silica and C18 columns.

### REFERENCES

The following references are relevant to certain aspects of the Examples presented herein. The entirety of each of these references, and their supporting information is incorporated herein by reference.
1. Tautenhahn, R., Bottcher, C. & Neumann, S. Highly sensitive feature detection for high resolution LC/MS. BMC Bioinformatics 9, 1-16 (2008).
2. Wang, M. et al. Sharing and community curation of mass spectrometry data with Global Natural Products Social Molecular Networking. Nat. Biotechnol. 34, 828-837 (2016).
3. Helf, M. J., Fox, B. W., Artyukhin, A. B., Zhang, Y. K. & Schroeder, F. C. Comparative metabolomics with Metaboseek reveals functions of a conserved fat metabolism pathway in C. elegans. Nat. Commun. 13, 782 (2022).
4. Shannon, P. et al. Cytoscape: A Software Environment for Integrated Models of Biomolecular Interaction Networks. Genome Res. 13, 2498-2504 (2003).

TABLE 3
HPLC-HRMS data for cysteamide derivatives

| | HR- | | | | | |
|---|---|---|---|---|---|---|
| | ESI(+/−) | | | | Retention | |
| | observed | | Calculated | Calculated | time | Schymanski |
| Compound | (m/z) | Ion | ion formula | m/z | (min) | rule |
| BU-MCY (1) | 162.0947 | [M + H]⁺ | C₇H₁₆ONS⁺ | 162.0947 | 3.5 | Level 1 |
| BU-MCYO (2) | 178.0899 | [M + H]⁺ | C₇H₁₆O₂NS⁺ | 178.0896 | 4.9 | Level 1 |
| BU-MCYO2 (3) | 194.0849 | [M + H]⁺ | C₇H₁₆O₃NS⁺ | 194.0845 | 5.3 | Level 1 |
| Taurobutyric acid (4) | 194.0494 | [M − H]⁻ | C₆H₁₂O₄NS⁻ | 194.0493 | 1.8 | Level 1 |
| CA-MCY (5) | 482.3294 | [M + H]⁺ | C₂₇H₄₈NO₄S⁺ | 482.3299 | 12.4 | Level 1 |
| CA-MCYO (6) | 498.3250 | [M + H]⁺ | C₂₇H₄₈NO₅S⁺ | 498.3248 | 10.8 | Level 1 |
| CA-MCYO2 (7) | 514.3207 | [M + H]⁺ | C₂₇H₄₈NO₆S⁺ | 514.3197 | 11.3 | Level 1 |
| BMCA-MCY (8) | 482.3294 | [M + H]⁺ | C₂₇H₄₈NO₄S⁺ | 482.3299 | 11.7 | Level 1 |
| CDCA-MCYO (9) | 482.3294 | [M + H]⁺ | C₂₇H₄₈NO₄S⁺ | 482.3299 | 11.9 | Level 1 |
| UDCA-MCYO (10) | 482.3294 | [M + H]⁺ | C₂₇H₄₈NO₄S⁺ | 482.3299 | 10.7 | Level 1 |
| DCA-MCYO (11) | 482.3294 | [M + H]⁺ | C₂₇H₄₈NO₄S⁺ | 482.3299 | 12.1 | Level 1 |
| KDCA-MCYO (12) | 496.3093 | [M + H]⁺ | C₂₇H₄₆NO₅S⁺ | 496.3095 | 10.1 | Level 1 |

The abbreviations in Table 3 refer to the chemical structures shown below:

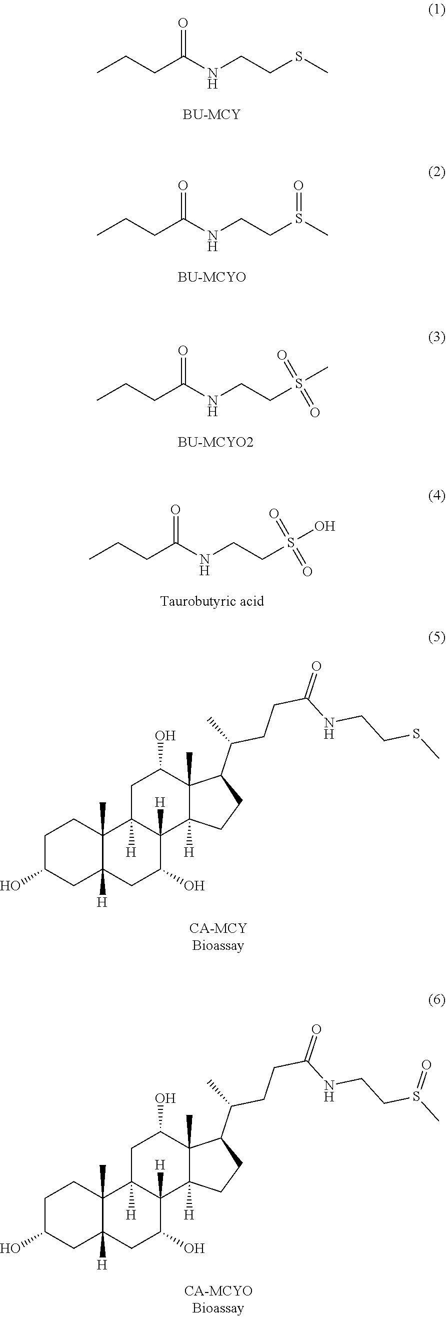

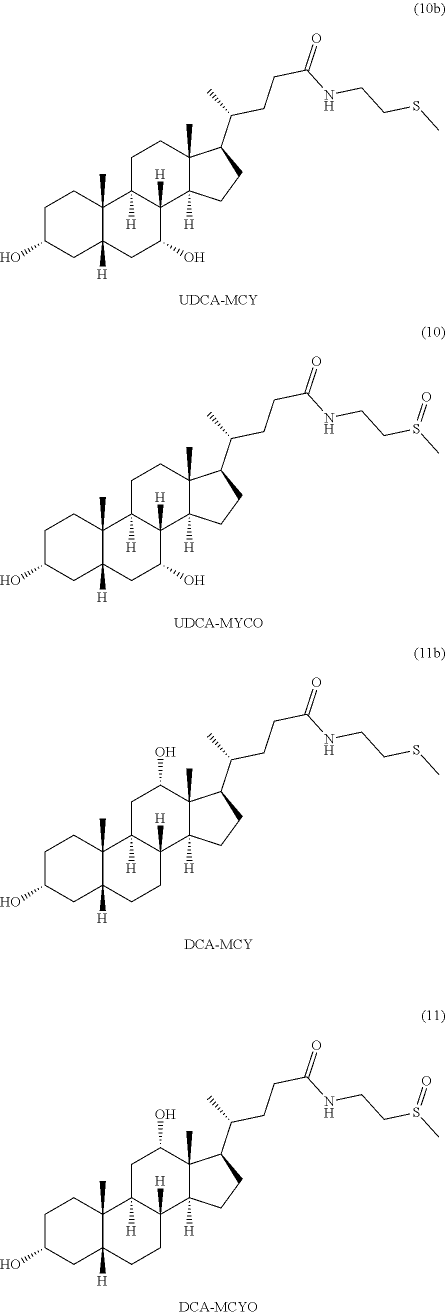

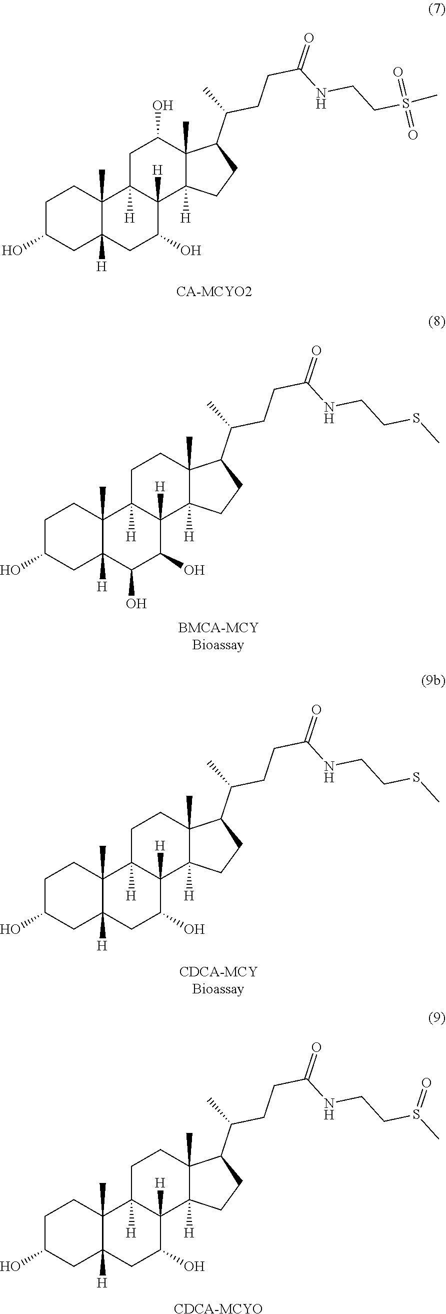

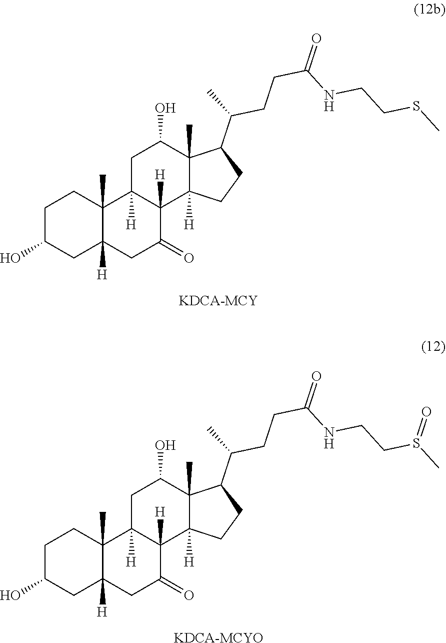

### Synthetic Procedures

### Example 1. Synthesis of BU-MCY (1) and BU-MCYO (2)

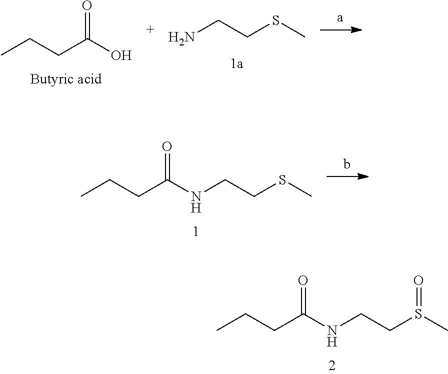

(a) To a stirred solution of butyric acid (Sigma, 77.5 μL, 1 mmol), 4-dimethylaminopyridine (488 mg, 4 mmol, 4.0 equiv.), and EDC-HCl (383 mg, 2 mmol, 2.0 equiv.) in 2 mL of DCM was added S-methylcysteamine (la, Sigma, 100 mg, 1.1 mmol, 1.1 equiv.). The reaction mixture was stirred at room temperature for 24 hr and concentrated in vacuo. Purification by flash chromatography on a reverse-phase column (C18) using a gradient of 0-50% ACN in 0.1% acetic acid afforded BU-MCY (1, 120 mg, 79%). BU-MCY (1): HRMS (ESI) m/z: [M+H]⁺ calcd for C₇H₁₆NOS⁺ 162.0947; found 162.0947.

(b) To a stirred solution of BU-MCY (Sigma, 60 mg, 0.37 mmol) in 1 mL of a mixture of 9:1 ACN:MeOH was added tert-butyl hydroperoxide solution (5.0-6.0 M in decane, Sigma, 0.5 ml, 2.5 mmol, 7 equiv.). The reaction mixture was stirred at room temperature for 24 hr and concentrated in vacuo. Purification by flash chromatography on a reverse-phase column (C18) using a gradient of 0-30% ACN in 0.1% acetic acid afforded BU-MCYO (2, 50 mg, 76%). MS (ESI) m/z: [M+H]⁺ calcd for C₇H₁₆NO₂S⁺ 178.0896; found 178.0899. See below for NMR spectroscopic data.

### Example 2. Synthesis of BU-MCYO2 (3) and taurobutyric acid (4)

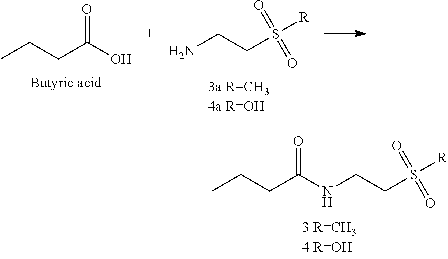

(c) To a stirred solution of butyric acid (Sigma, 77.5 μL, 1 mmol), 4-dimethylaminopyridine (366 mg, 3 mmol, 6.0 equiv.), and EDC-HCl (192 mg, 1 mmol, 2.0 equiv.) in 1 mL of DMF was added 2-(methylsulfonyl)ethanamine hydrochloride (3a, Synthonix, 240 mg, 1.5 mmol, 1.5 equiv.). The reaction mixture was stirred at room temperature for 24 hr and concentrated in vacuo. Purification by flash chromatography on a reverse-phase column (C18) using a gradient of 0-30% ACN in 0.1% acetic acid afforded BU-MCYO2 (3, 180 mg, 93%). The same procedure was performed using taurine (4a, Sigma, 242 mg, 1.5 mmol) instead of 2-(methylsulfonyl)ethanamine hydrochloride for synthesis of taurobutyric acid (4, 78 mg, 40%). BU-MCYO2 (3): MS (ESI) m/z: [M+H]⁺ calcd for C₇H₁₆NO₃S⁺ 194.0845; found 194.0849. Taurobutyric acid (4): MS (ESI) m/z: [M−H]⁻ calcd for C₆H₁₂NO₄S⁻ 194.0493; found 194.0494. See below for NMR spectroscopic data.

### Example 3. Synthesis of BA(Bile Acid)-MCY (5, 8, and 9b-12b) and BA-MCYO (6, and 9-12)

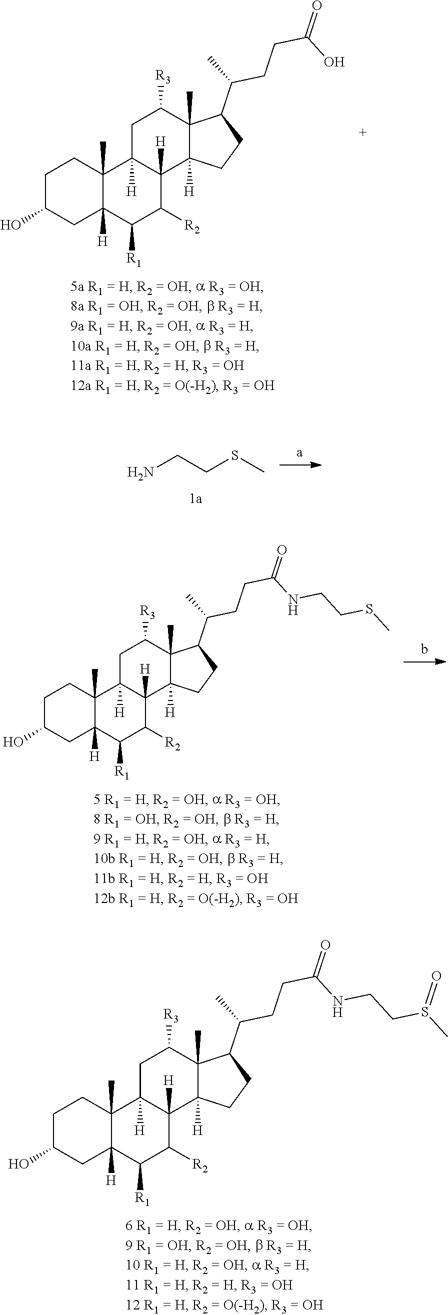

(a) To a stirred solution of cholic acid (5a, Sigma, 204 mg, 0.5 mmol), 4-dimethylaminopyridine (244 mg, 2 mmol, 4.0 equiv.), and EDC-HCl (192 mg, 1 mmol, 2.0 equiv.) in 1 mL of DCM was added S-methylcysteamine (la, Sigma, 55 mg, 0.6 mmol, 1.1 equiv.). The reaction mixture was stirred at room temperature for 24 hr and concentrated in vacuo. Purification by flash chromatography on a reverse-phase column (C18) using a gradient of 30-80% ACN in 0.1% acetic acid afforded CA-MCY (5, 200 mg, 82%). The same procedure was performed using P-muricholic acid (8a, Sigma, 15 mg, 0.037 mmol), chenodeoxycholic acid (9a, Sigma, 186 mg, 0.5 mmol), ursodeoxycholic acid (10a, Sigma, 186 mg, 0.5 mmol), deoxycholic acid (11a, Sigma, 93 mg, 0.25 mmol), or 7-ketodeoxycholic acid (12a, Sigma, 20 mg, 0.05 mmol) instead of cholic acid for synthesis of OMCA-MCY (8, 17 mg, 94%), CDCA-MCY (9b, 200 mg, 82%), UDCA-MCY (10b, 200 mg, 82%), DCA-MCY (11b, 95 mg, 79%), and KDCA-MCY (12b, 21 mg, 86%), respectively. CA-MCY (5): HRMS (ESI) m/z: [M+H]⁺ calcd for C₂₇H₄₈NO₄S⁺ 482.3299; found 482.3294. OMCA-MCY (8): HRMS (ESI) m/z: [M+H]⁺ calcd for C₂₇H₄₈NO₄S⁺ 482.3299; found 482.3294. CDCA-MCY (9b): HRMS (ESI) m/z: [M+H]⁺ calcd for C₂₇H₄₈NO₄S⁺ 482.3299; found 482.3294. UDCA-MCY (10b): HRMS (ESI) m/z: [M+H]⁺ calcd for C₂₇H₄₈NO₄S⁺ 482.3299; found 482.3294. DCA-MCY (11b): HRMS (ESI) m/z: [M+H]⁺ calcd for C₂₇H₄₈NO₄S⁺ 482.3299; found 482.3294. KDCA-MCY (12b): HRMS (ESI) m/z: [M+H]⁺ calcd for C₂₇H₄₆NO₄S⁺ 480.3142; found 480.3137. See below for NMR spectroscopic data.

(b) To a stirred solution of CA-MCY (5, 90 mg, 0.19 mmol) in 1 mL of DCM was added tert-butyl hydroperoxide solution 5.0-6.0 M in decane (Sigma, 0.3 ml, 1.5 mmol, 7 equiv.). The reaction mixture was stirred at room temperature for 24 hr and concentrated in vacuo. Purification by flash chromatography on a reverse-phase column (C18) using a gradient of 20-80% ACN in 0.1% acetic acid afforded CA-MCYO (6, 80 mg, 85%). The same procedure was performed using CDCA-MCY (9b, 48 mg, 0.1 mmol), UDCA-MCY (10b, 48 mg, 0.1 mmol), DCA-MCY (11b, 24 mg, 0.05 mmol), or KDCA-MCY (12b, 12 mg, 0.025 mmol) instead of CA-MCY for synthesis of CDCA-MCYO (9, 44 mg, 89%), UDCA-MCYO (10, 42 mg, 85%), DCA-MCYO (11, 19 mg, 80%), and KDCA-MCYO (12, 11 mg, 88%), respectively. CA-MCYO (6): MS (ESI) m/z: [M+H]⁺ calcd for C₂₇H₄₈NO₆S 498.3248; found 498.3250. CDCA-MCYO (9): MS (ESI) m/z: [M+H]⁺ calcd for C₂₇H₄₈NO₆S⁺ 498.3248; found 498.3250. UDCA-MCYO (10): MS (ESI) m/z: [M+H]⁺ calcd for C₂₇H₄₈NO₆S⁺ 498.3248; found 498.3250. DCA-MCYO (11): MS (ESI) m/z: [M+H]⁺ calcd for C₂₇H₄₈NO₆S⁺ 498.3248; found 498.3250. KDCA-MCYO (12): MS (ESI) m/z: [M+H]⁺ calcd for C₂₇H₄₆NO₆S⁺ 496.3091; found 496.3093. See below for NMR spectroscopic data.

### Example 4. Synthesis of CA-MCYO2 (7)

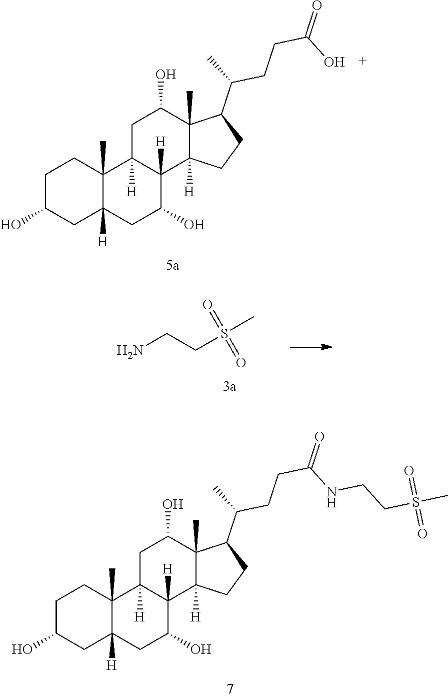

To a stirred solution of cholic acid (5a, Sigma, 204 mg, 0.5 mmol), 4-dimethylaminopyridine (366 mg, 3 mmol, 6.0 equiv.), and EDC-HCl (192 mg, 1 mmol, 2.0 equiv.) in 1 mL of DMF was added 2-(methylsulfonyl)ethanamine hydrochloride (3a, Synthonix, 96 mg, 0.6 mmol, 1.1 equiv.). The reaction mixture was stirred at room temperature for 24 hr and concentrated in vacuo. Purification by flash chromatography on a reverse-phase column (C18) using a gradient of 30-80% ACN in 0.1% aqueous acetic acid afforded CA-MCYO2 (7, 180 mg, 75%). HRMS (ESI) m/z: [M+H]⁺ calcd for C₂₇H₄₈NO₆S⁺ 514.3197; found 514.3197. See below for NMR spectroscopic data.

### Example 5. Assay for Activity Against Human FXR

Tauro-β-muricholic acid (Sigma) and synthetic MCY conjugates including CA-MCY, CA-MCYO, CDCA-MCY, and PMCA-MCY were submitted for a cell-based assay to test for activity as an antagonist or agonist of human farnesoid X receptor (FXR). Known ligands, GW4064 and DY268 were used as controls. Compound activity was detected by chemiluminescent signals indicating ligand binding that induces FXR activation, translocation, and co-activator interaction. Agonist mode measures percentage activity relative to maximum value activated by GW4064 (100% activation). For antagonist determination, cells were pre-incubated with antagonist followed by agonist challenge at the GW4064 (0.37 μM, EC80). For agonist mode assays, percentage activity was calculated using the following formula: activity (%)=100×(mean RLU of test sample−mean RLU of vehicle control)/(mean MAX control ligand −mean RLU of vehicle control). For antagonist mode assays, percentage inhibition was calculated using the following formula: inhibition (%)=100×(1−(mean RLU of test sample−mean RLU of vehicle control)/(mean RLU of EC80 control−mean RLU of vehicle control).

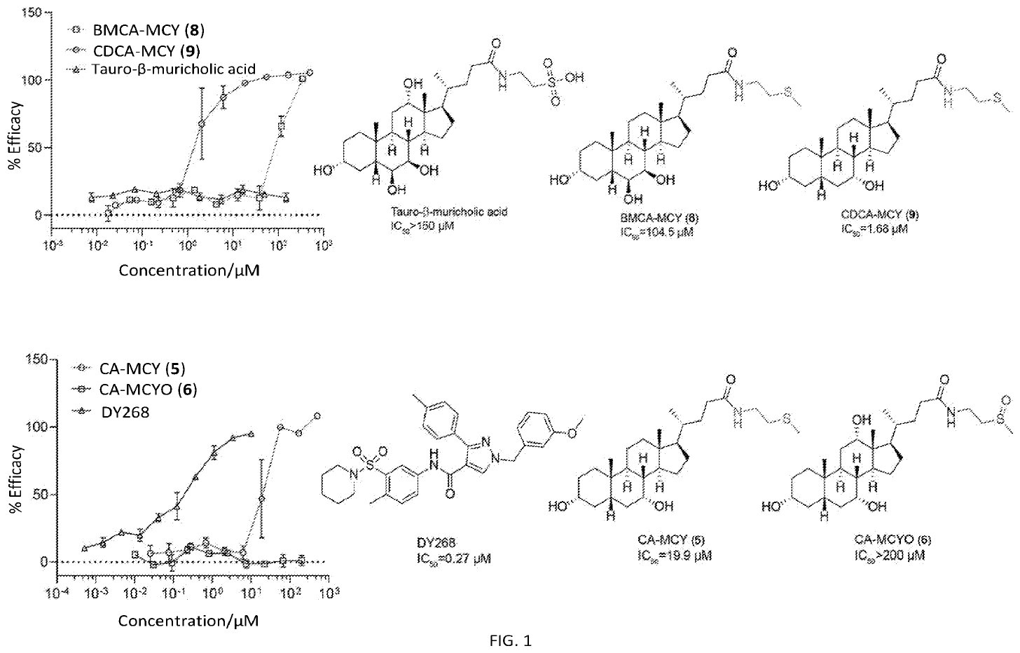

The results of this assay are shown in FIG. 1. These data show that the tested BA cysteamides are potent antagonists of FXR. Because FXR antagonistism is known to reduce bile acid production and to modulate cholesterol production/transport as well as fat metabolism, these results are evidence that the compounds shown in FIG. 1 and other related molecules described herein have utility in therapies for a wide range of health disorders and diseases related to bile acid production, cholesterol and fat.

### Example 6. NMR Spectroscopic Data of Certain Compounds of the Invention

### Example 6.1 ¹H (600 MHz) and ¹³C (151 MHz) NMR Spectroscopic Data for BU-MCY (Structure Below) in Methanol-d₄

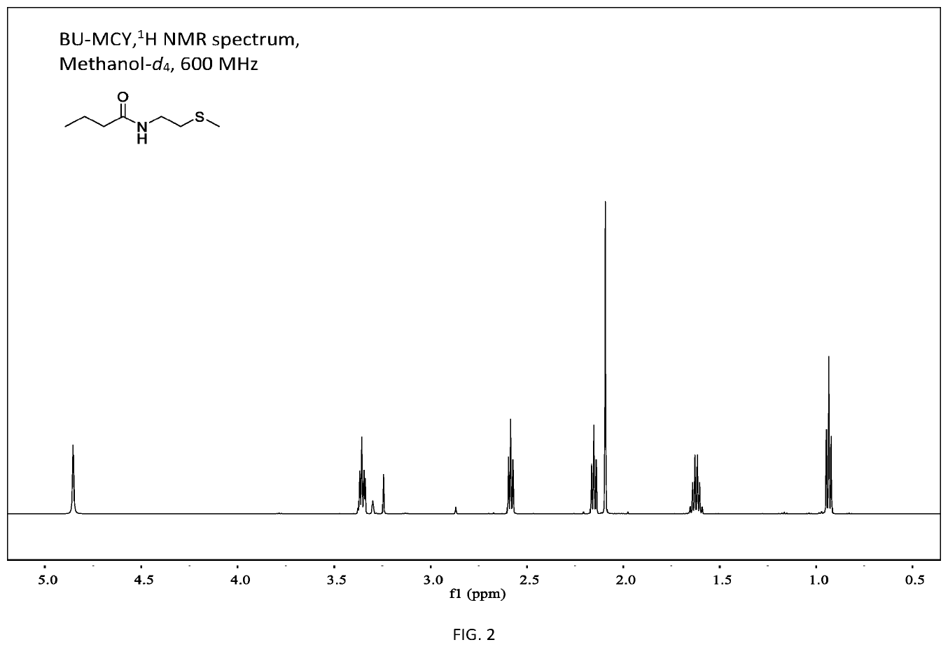

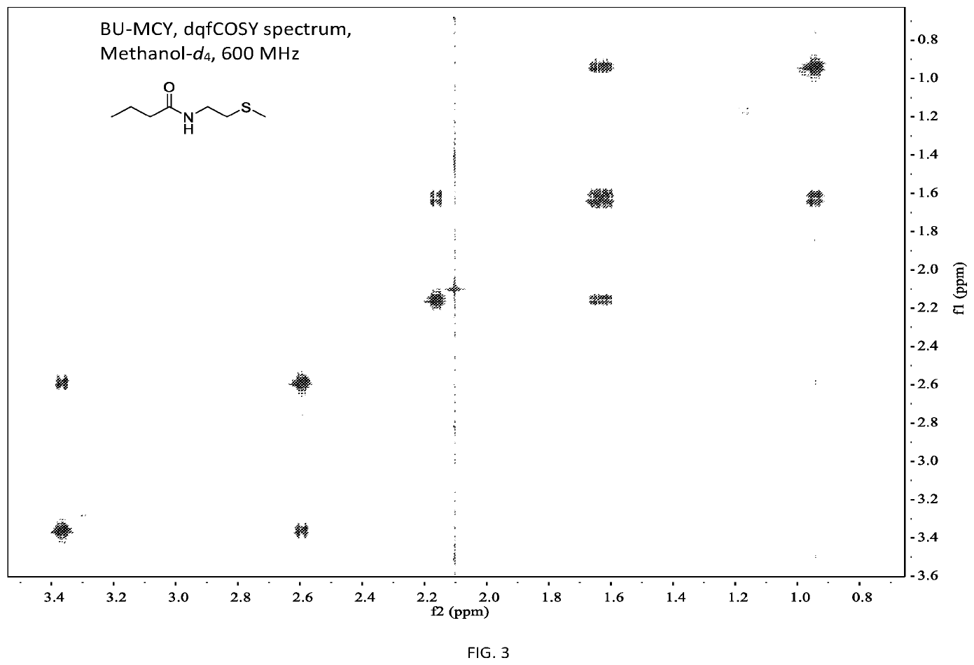

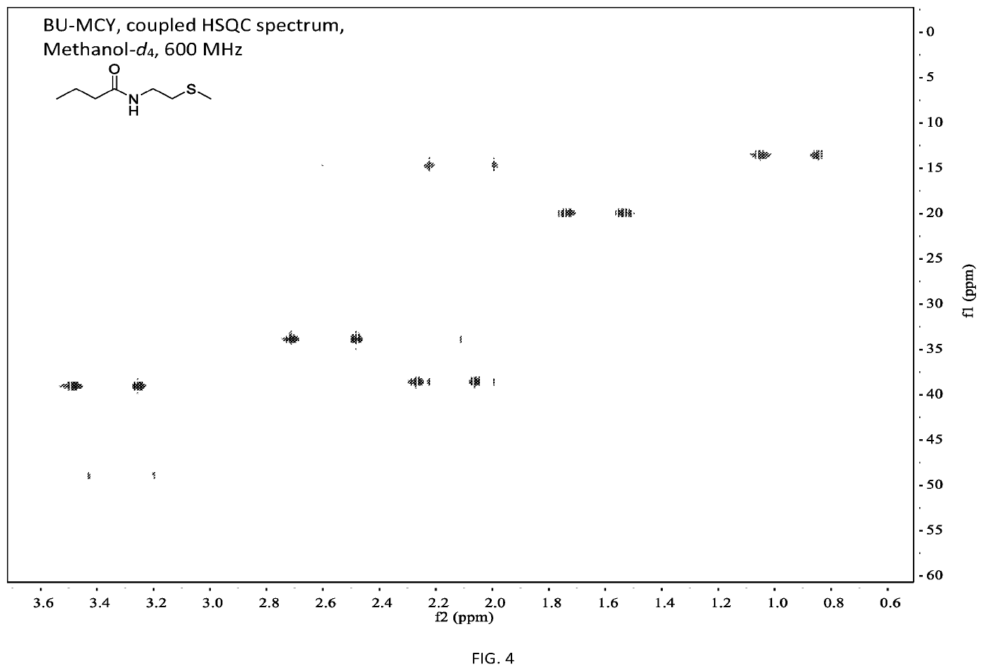

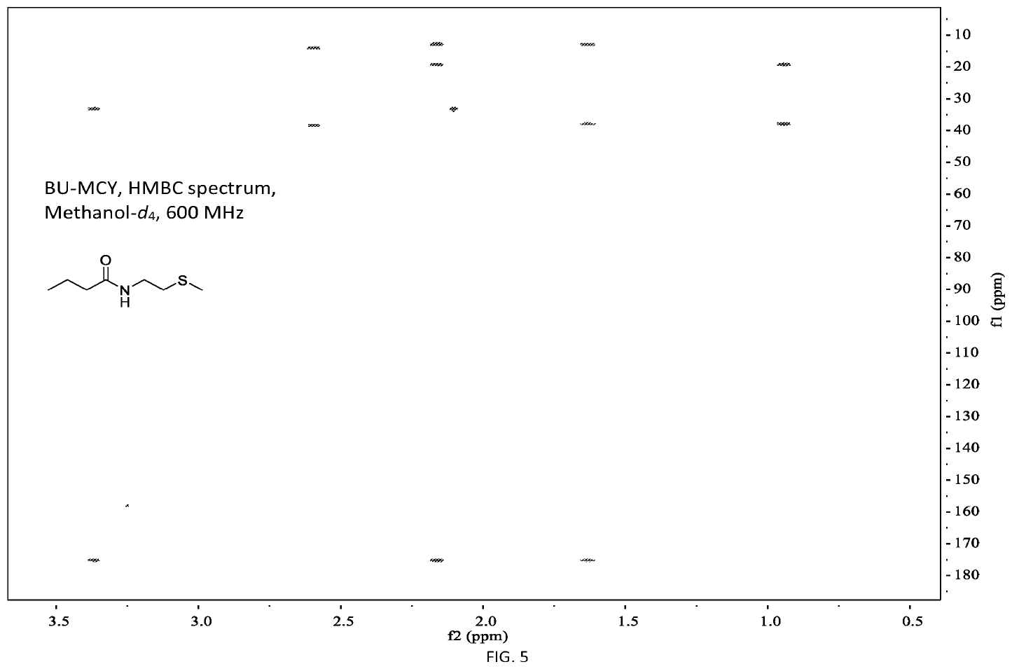

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The ¹H, dqfCOSY, HSQC and HMBC spectra of BU-MCY are shown in FIG. 2 through FIG. 5.

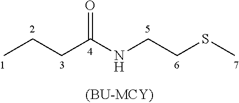

| No. | δc | Proton | δH (J_{HH}[Hz]) | HMBC |
|---|---|---|---|---|
| 1 | 13.9 | 1-H | 0.94 (J_{1,2} = 7.3 Hz) | 2, 3 |
| 2 | 20.1 | 2-H | 1.63 (J_{2,3} = 7.3 Hz, J_{2,1} = 7.3 Hz) | 1, 3 |
| 3 | 38.7 | 3-H | 2.16 (J_{3,2} = 7.3 Hz) | 1, 2 |
| 4 | 175.9 | | | 2, 3, 5 |
| 5 | 39.2 | 5-H | 3.37 (J_{5,6} = 7.0 Hz) | 6 |
| 6 | 34.0 | 6-H | 2.59 (J_{6,5} = 7.0 Hz) | 5, 7 |
| 7 | 14.8 | 7-H | 2.10 | 6 |

### Example 6.2 ¹H (600 MHz) and ¹³C (151 MHz) NMR Spectroscopic Data for BU-MCYO (Structure Below) in Methanol-d₄

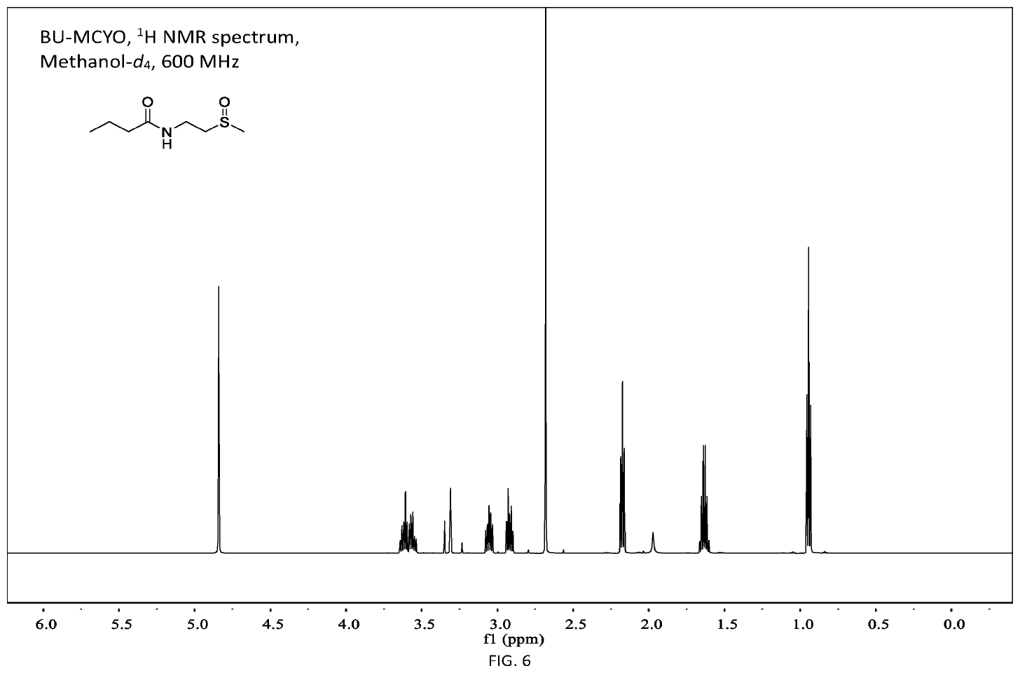

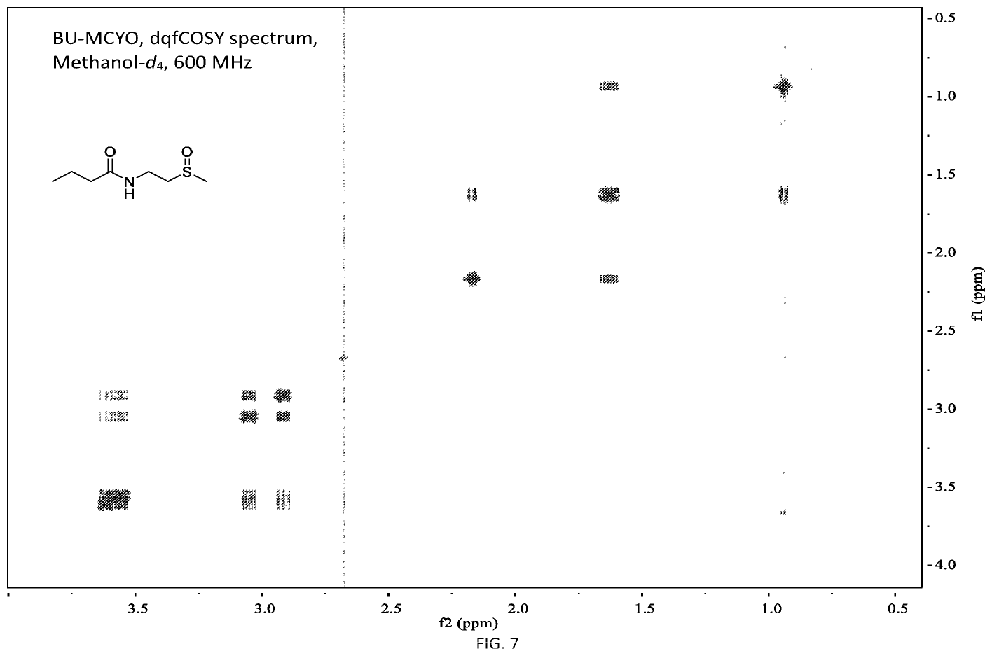

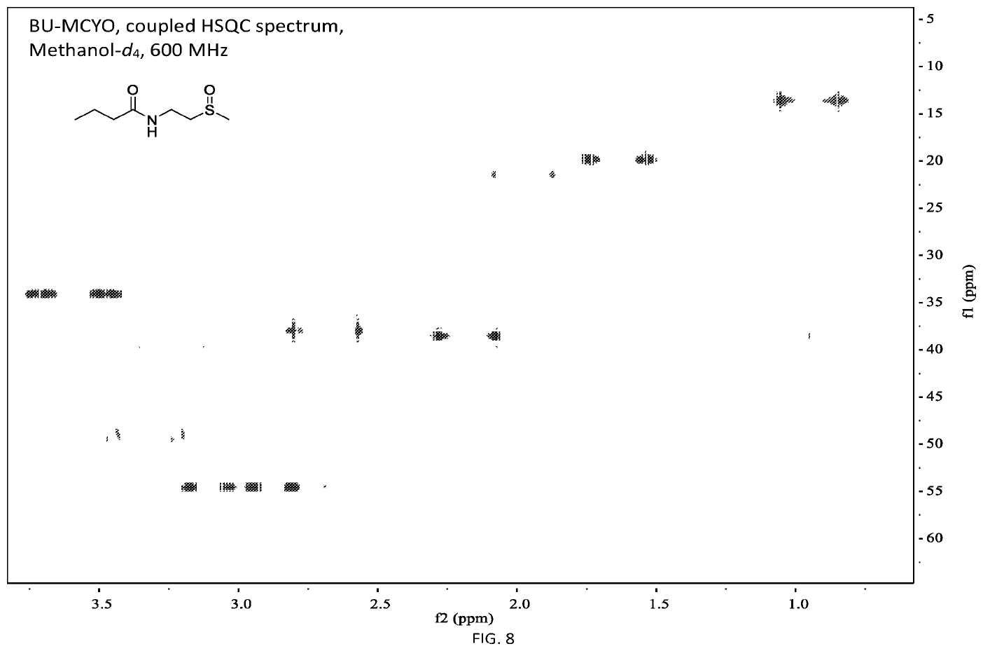

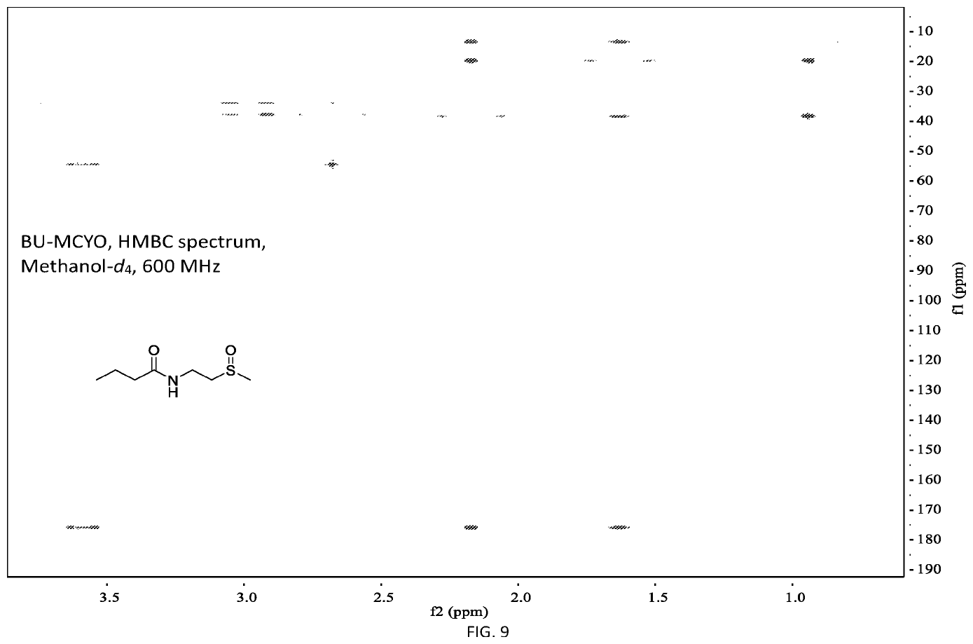

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of BU-MCYO are shown in FIG. 6 through FIG. 9.

(BU-MCYO)

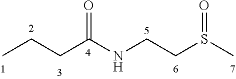

| No. | δc | Proton | δH (J_{HH}[Hz]) | HMBC |
|---|---|---|---|---|
| 1 | 13.7 | 1-H | 0.94 (J_{1,2} = 7.3 Hz) | 2, 3 |
| 2 | 20.0 | 2-H | 1.63 (J_{2,3} = 7.3 Hz, J_{2,1} = 7.3 Hz) | 1, 3 |
| 3 | 38.6 | 3-H | 2.17 (J_{3,2} = 7.3 Hz) | 1, 2 |
| 4 | 176.1 | | | 2, 3, 5a, 5b |
| 5 | 34.2 | 5-Ha | 3.57 (J_{5a,6a} = 6.0 Hz, J_{5a,6b} = 6.5 Hz, J_{5a,5b} = 12.5 Hz) | 6, 7 |
| | | 5-Hb | 3.61 (J_{5b,6a} = 6.0 Hz, J_{5b,6b} = 6.5 Hz, J_{5b,5a} = 12.5 Hz) | |
| 6 | 54.6 | 6-Ha | 2.92 (J_{6a,6b} = 12.8 Hz, J_{6a,5a} = 6.0 Hz, J_{6a,5b} = 6.0 Hz) | 5a, 5b, 7 |
| | | 6-Hb | 3.05 (J_{6b,6a} = 12.8 Hz, J_{6b,5a} = 6.5 Hz, J_{6b,5b} = 6.5 Hz) | |
| 7 | 38.1 | 7-H | 2.68 | 6 |

### Example 6.3 ²H (600 MHz) and ¹³C (151 MHz) NMR Spectroscopic Data for BU-MCYO2 (Structure Below) in Methanol-d₄

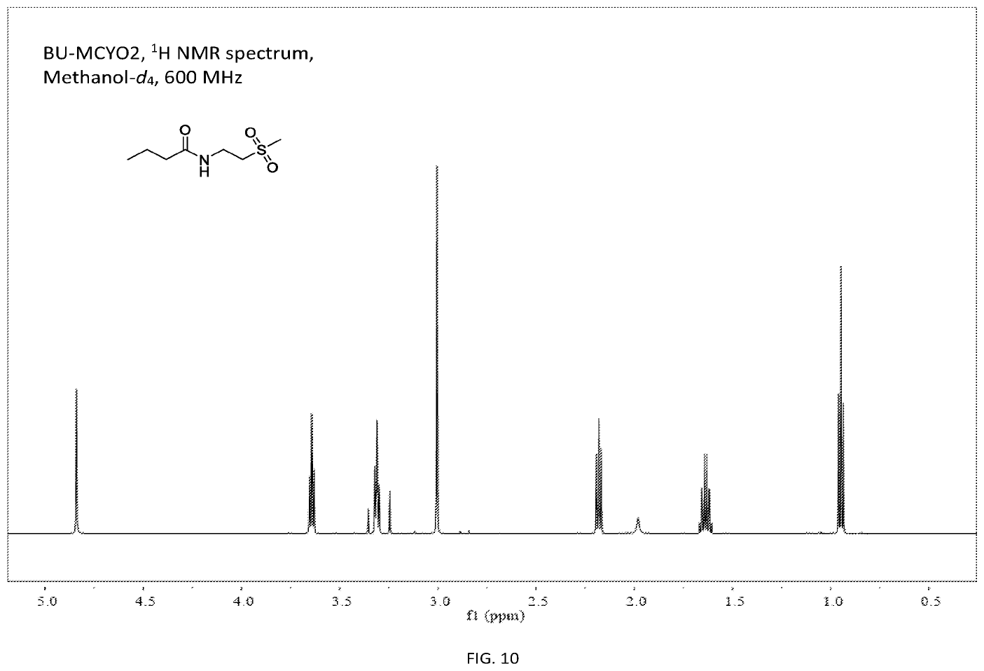

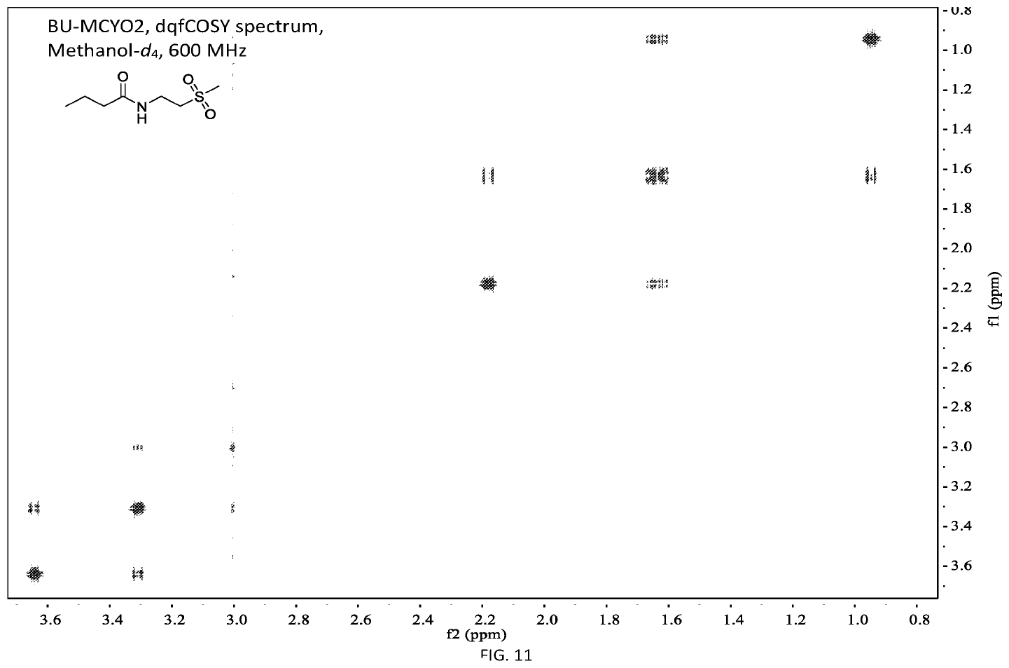

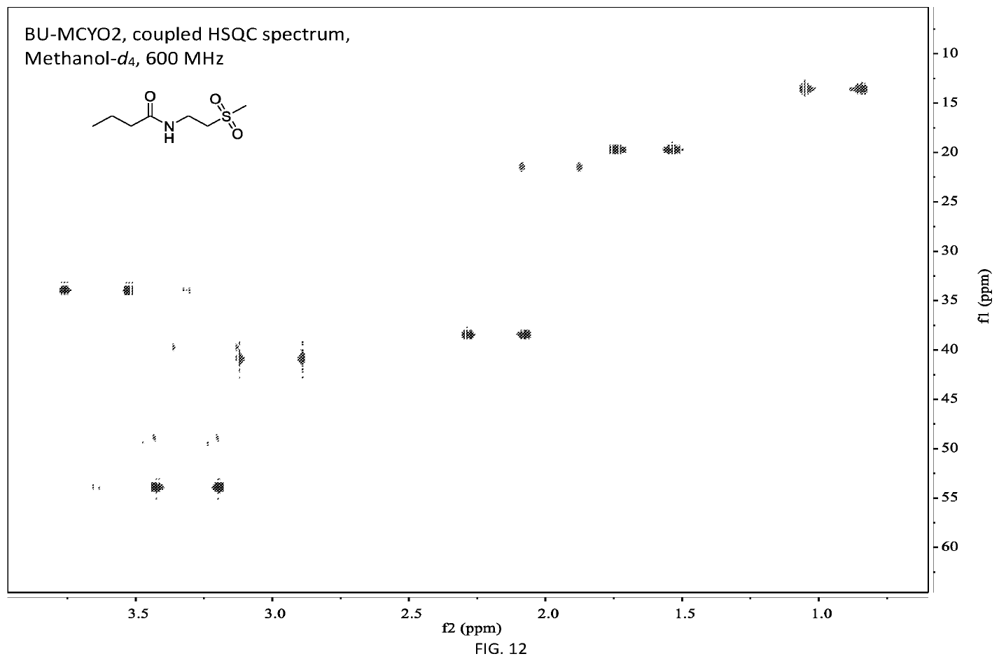

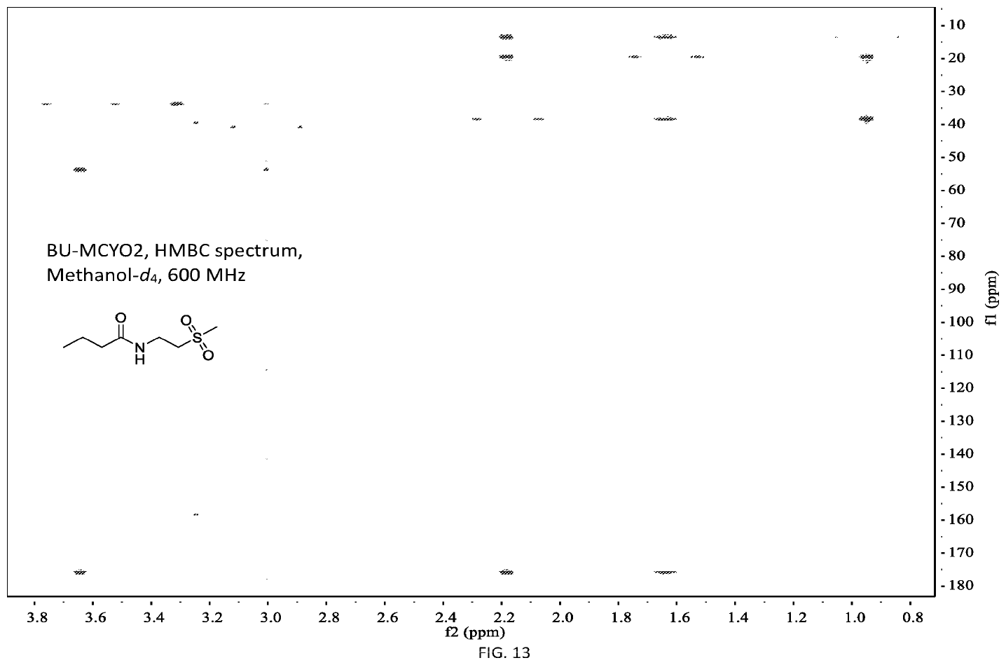

Chemical shifts were referenced to δ(CHD₂OD)=3.31 and δ(¹³CHD₂OD)=49.0. ¹³C chemical shifts were determined via MBC and HSQC spectra. ¹H, ¹H-J-coupling constants were determined from the acquired ¹H or dqfCOSY spectra. TMBC correlations are from the proton(s) stated to the indicated ¹³C atom. The corresponding ¹H, dqfCOSY, HSQC and HMBC spectra of BU-MCYO2 are shown in FIG. 10 through FIG. 13.

(BU-MYCO2)

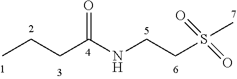

| No. | δc | Proton | δH (J_{HH}[Hz]) | HMBC |
|---|---|---|---|---|
| 1 | 13.7 | 1-H | 0.94 (J_{1,2} = 7.3 Hz) | 2, 3 |
| 2 | 19.9 | 2-H | 1.63 (J_{2,3} = 7.3 Hz, | 1, 3 |
| | | | J_{2,1} = 7.3 Hz) | |
| 3 | 38.6 | 3-H | 2.17 (J_{3,2} = 7.3 Hz) | 1, 2 |
| 4 | 176.1 | | | 2, 3, 5 |
| 5 | 34.1 | 5-H | 3.63 (J_{5,6} = 6.5 Hz) | 6, 7 |
| 6 | 54.1 | 6-H | 3.30 (J_{6,5} = 6.5 Hz) | 5, 7 |
| 7 | 41.0 | 7-H | 3.00 | |

### Example 6.4 ¹H (600 MHz) and ¹³C (151 MHz) NMR Spectroscopic Data for Taurobutyric Acid in Methanol-d₄

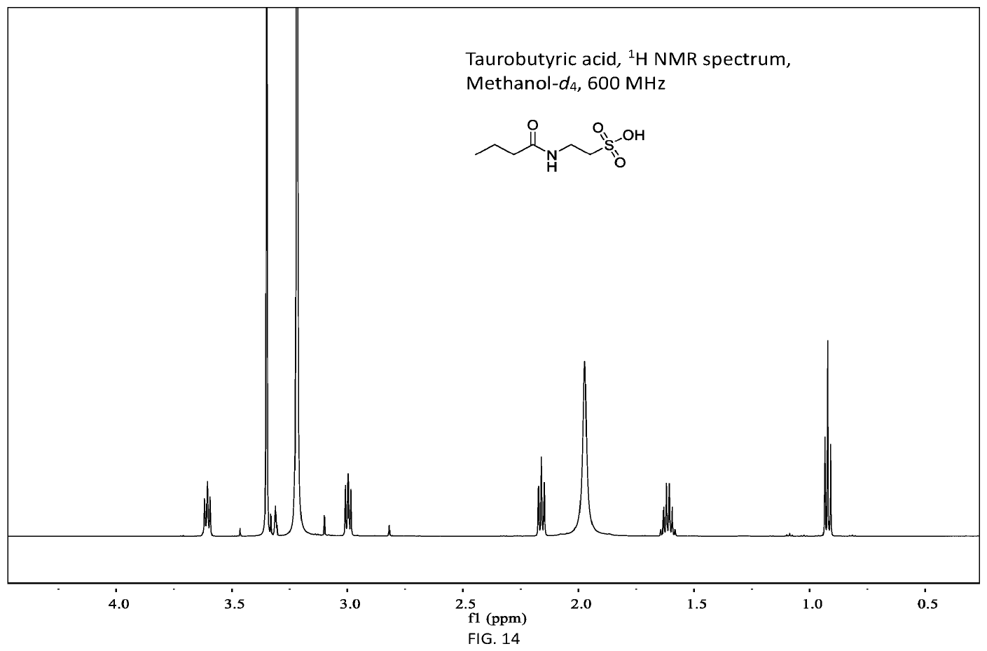

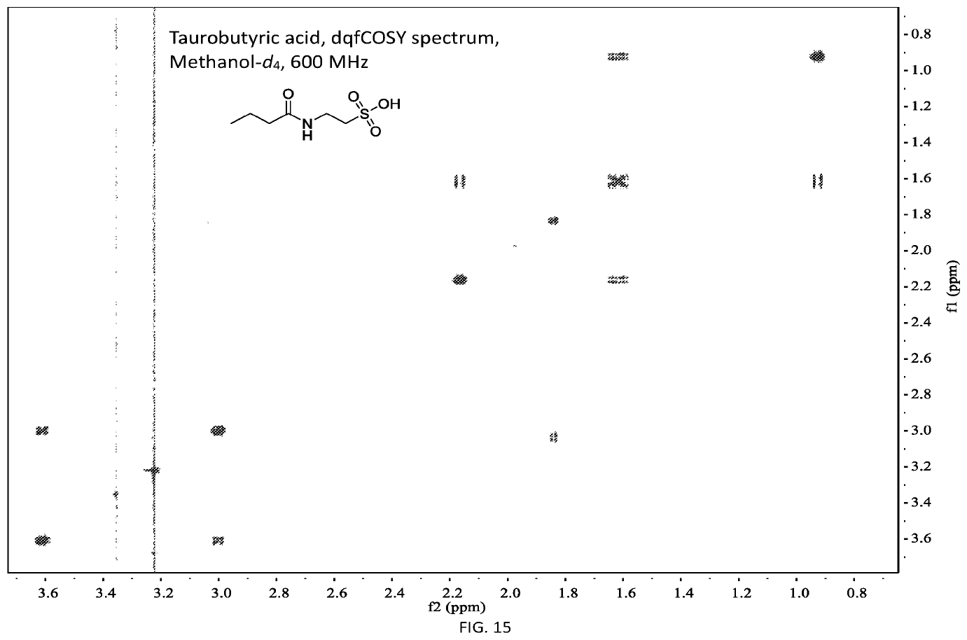

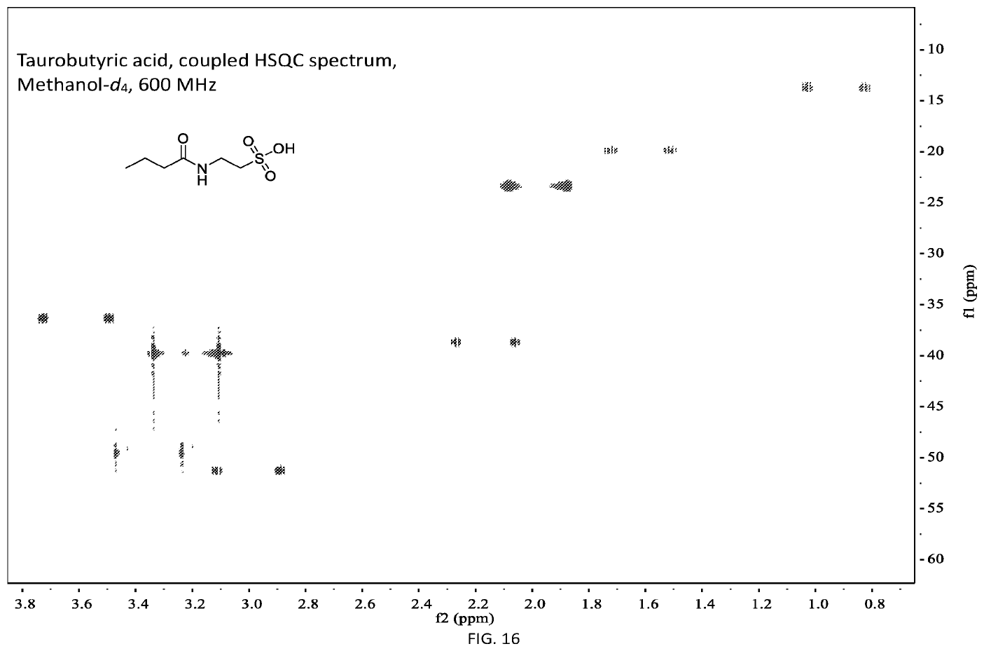

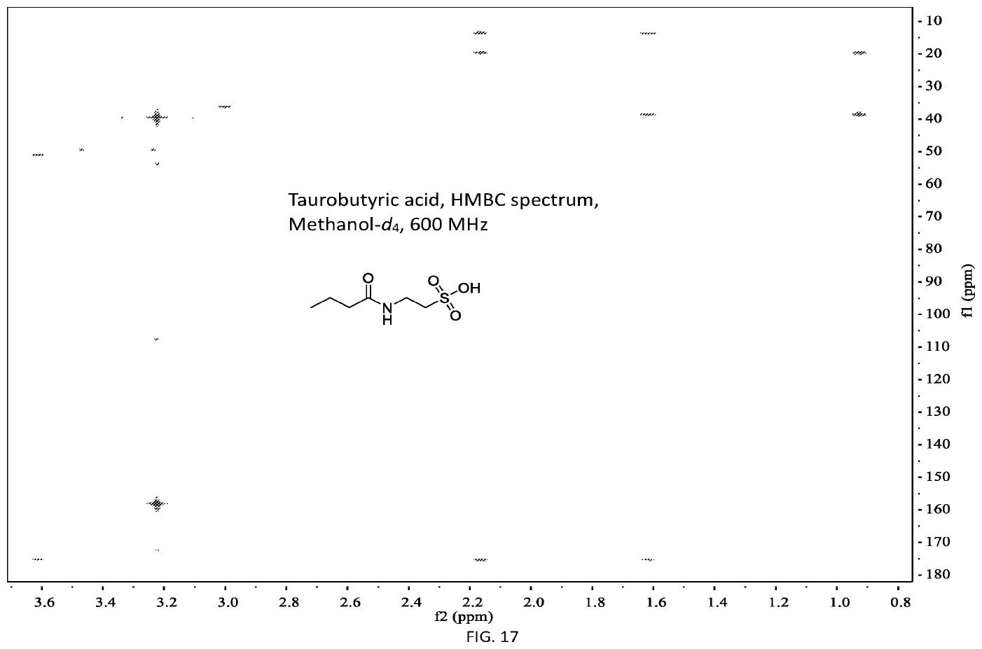

Chemical shifts were referenced to δ(CHD₂OD)=3.31 and δ(¹³CHD₂OD)=49.0. ¹³C chemical shifts were determined via HMBC and HSQC spectra. ¹H, ¹H₁-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of Taurobutyric acid are shown in FIG. 14 through FIG. 17.

(Taurobutyric acid)

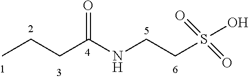

| No. | δc | Proton | δH (J_{HH}[Hz]) | HMBC |
|---|---|---|---|---|
| 1 | 13.8 | 1-H | 0.92 (J_{1,2} = 7.3 Hz) | 2, 3 |
| 2 | 19.9 | 2-H | 1.61 (J_{2,3} = 7.3 Hz, | 1, 3 |
| | | | J_{2,1} = 7.3 Hz) | |
| 3 | 38.7 | 3-H | 2.16 (J_{3,2} = 7.3 Hz) | 1, 2 |
| 4 | 175.3 | | | 2, 3, 5 |
| 5 | 36.4 | 5-H | 3.61 (J_{5,6} = 7.0 Hz) | 6 |
| 6 | 51.4 | 6-H | 3.00 (J_{6,5} = 7.0 Hz) | 5 |

### Example 6.5 ¹H (600 MHz) and ¹³C (151 MHz) NMR Spectroscopic Data for CA-MCY (Structure Below) in Methanol-d₄

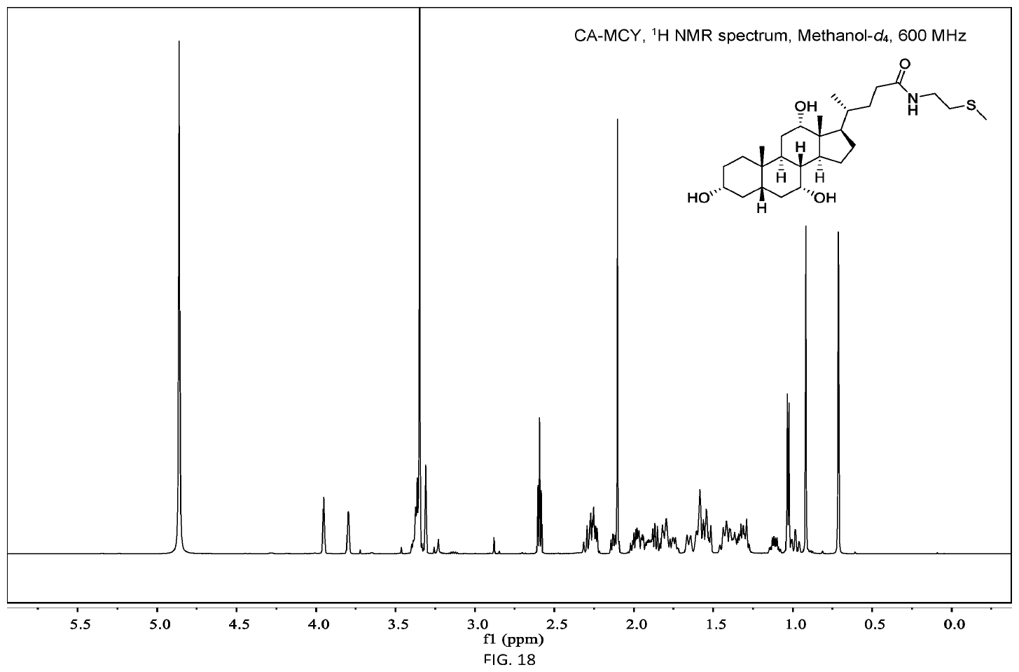

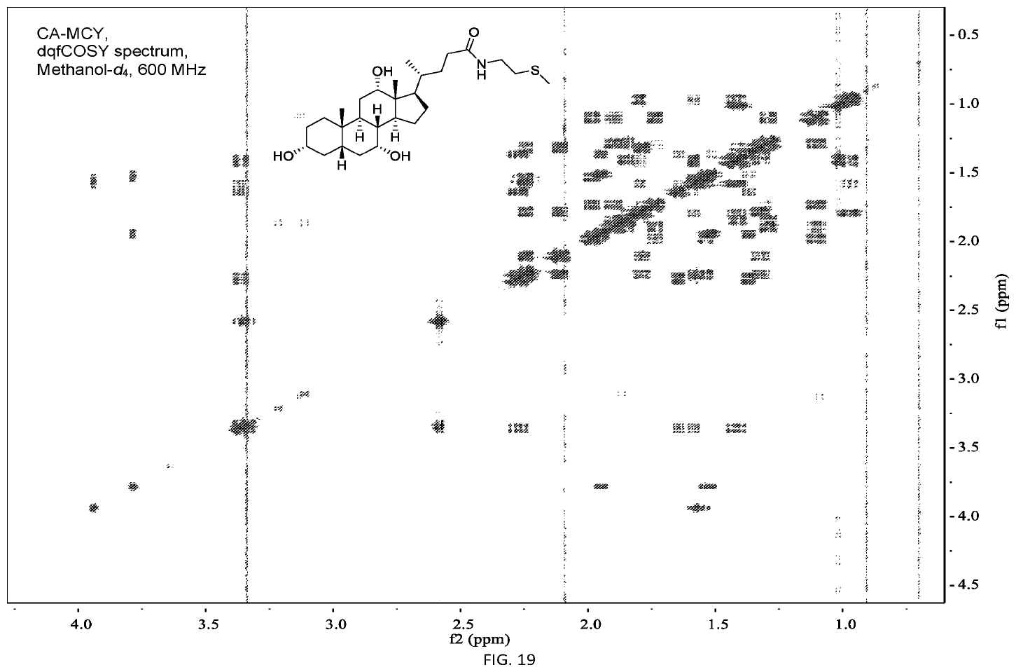

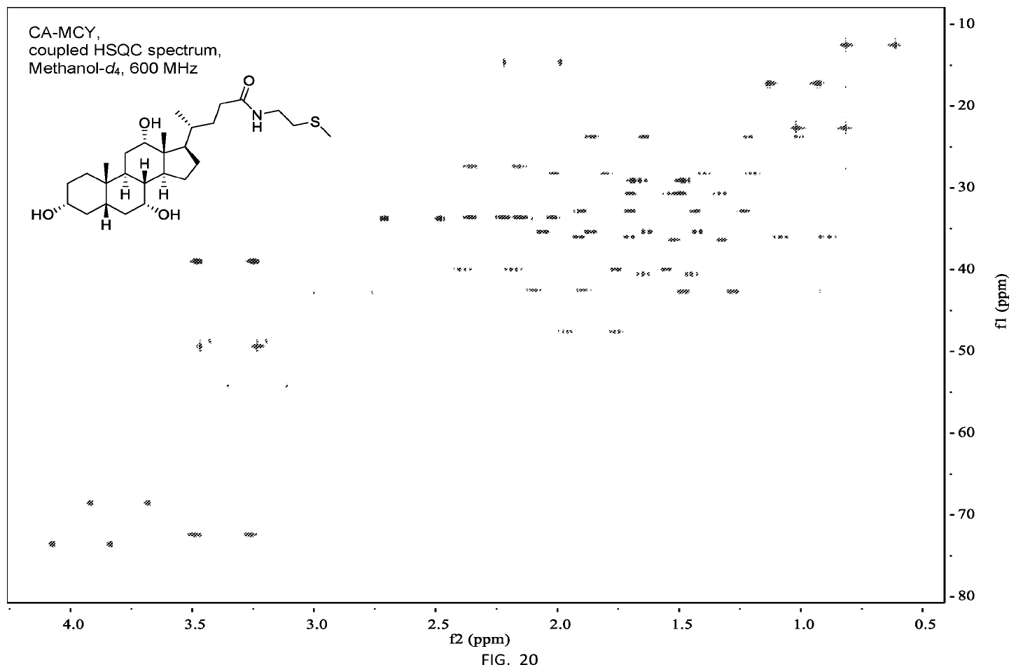

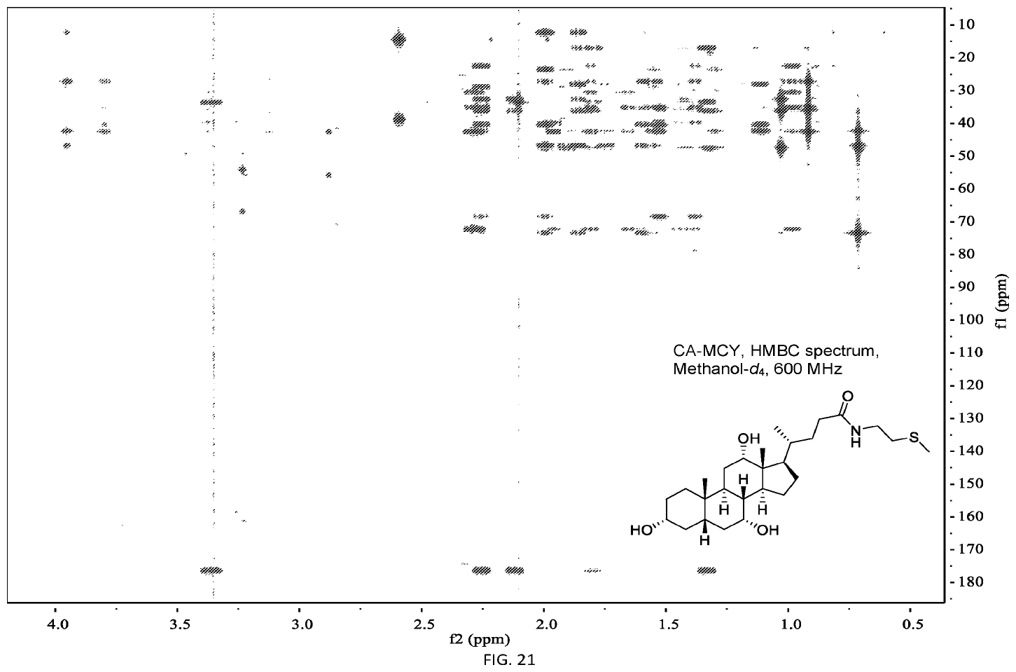

Chemical shifts were referenced to δ(CHD₂OD)=3.31 and δ(¹³CHD₂OD)=49.0. ¹³C chemical determined from the acquired ¹H or dqfCOSY spectra. HMBC correlations are from the proton(s) stated to the indicated ¹³C atom. The corresponding ¹H, dqfCOSY, HSQC and HMBC spectra of CA-MCY are shown in FIG. 18 through FIG. 21.

(CA-MCY)

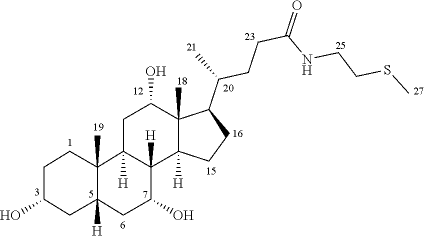

| No. | δc | Proton | δH (J_{HH}[Hz]) | HMBC |
|---|---|---|---|---|
| 1 | 36.2 | 1-Ha | 0.98 (J_{1a,2a} = 13.8 Hz, J_{1a,2b} = 3.3 Hz, J_{1a,1b} = 13.8 Hz) | 19 |
| | | 1-Hb | 1.81 (m) | |
| 2 | 30.9 | 2-Ha | 1.44 (m) | 1a, 4b |
| | | 2-Hb | 1.58 (m) | |
| 3 | 72.6 | 3-H | 3.38 (m) | 1, 4b |
| 4 | 40.2 | 4-Ha | 1.65 (m) | 6a |
| | | 4-Hb | 2.27 (m) | |
| 5 | 42.9 | 5-H | 1.38 (m) | 1, 4b, 6, 19 |
| 6 | 35.6 | 6-Ha | 1.53 (m) | 4b |
| | | 6-Hb | 1.96 (m) | |
| 7 | 68.8 | 7-H | 3.80 (m) | 6a, 8 |
| 8 | 40.8 | 8-H | 1.54 (m) | 6a, 7, 9, 11, 14, 15a |
| 9 | 27.6 | 9-H | 2.26 (m) | 1a, 7, 8, 11, 12, 14, 19 |
| 10 | 35.6 | | | 1, 4b, 5, 6a, 9, 19 |
| 11 | 29.3 | 11-H | 1.60 (m) | 9 |
| 12 | 73.8 | 12-H | 3.94 (m) | 11, 14, 17, 18 |
| 13 | 47.2 | | | 11, 12, 14, 17, 18 |
| 14 | 42.7 | 14-H | 1.99 (m) | 8, 12, 15a, 18 |
| 15 | 24.0 | 15-Ha | 1.11 (J_{15a,16a} = 11.5 Hz, J_{15a,16b} = 5.5 Hz, J_{15a,15b} = 11.5 | 14 |
| | | | Hz, J_{15a,14} = 11.5 Hz) | |
| | | 15-Hb | 1.76 (m) | |
| 16 | 28.5 | 16-Ha | 1.30 (m) | 15a, 17 |
| | | 16-Hb | 1.90 (m) | |
| 17 | 47.8 | 17-H | 1.86 (m) | 18, 21 |
| 18 | 12.7 | 18-H | 0.71 | 12, 14, 17 |
| 19 | 22.9 | 19-H | 0.92 | 1a, 9 |
| 20 | 36.6 | 20-H | 1.42 (m) | 17, 21, 22a |
| 21 | 17.5 | 21-H | 1.02 (J_{21,20} = 6.6 Hz) | 22 |
| 22 | 33.1 | 22-Ha | 1.33 (m) | 21, 23 |
| | | 22-Hb | 1.80 (m) | |
| 23 | 33.9 | 23-Ha | 2.12 (m) | 22a |
| | | 23-Hb | 2.24 (m) | |
| 24 | 176.7 | | | 22a, 23, 25 |
| 25 | 39.2 | 25-H | 3.37 (J_{25,26} = 7.0 Hz) | 26 |
| 26 | 34.0 | 26-H | 2.59 (J_{26,25} = 7.0 Hz) | 25, 27 |
| 27 | 14.9 | 27-H | 2.10 | 26 |

### Example 6.6 ¹H (600 MHz) and ¹³C (151 MHz) NMR Spectroscopic Data for CA-MCYO2 (Structure Below) in Methanol-d₄

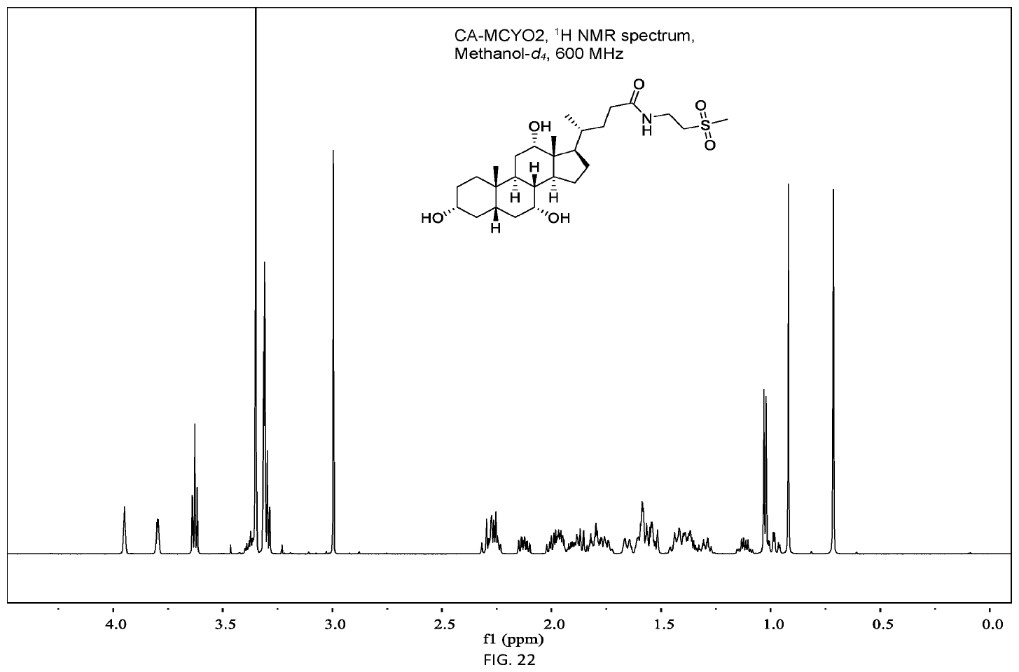

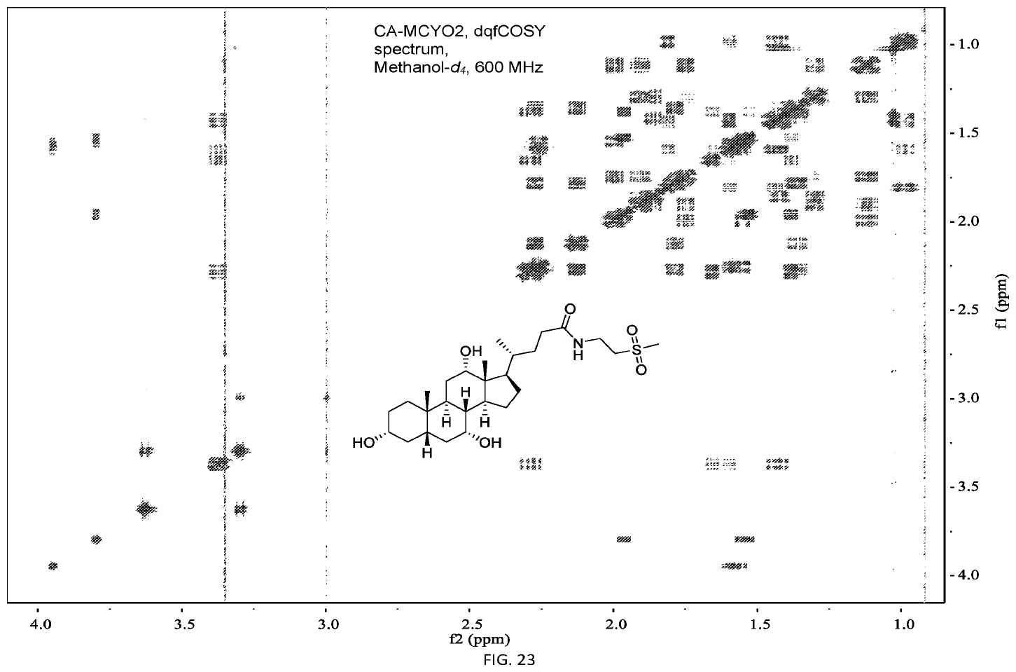

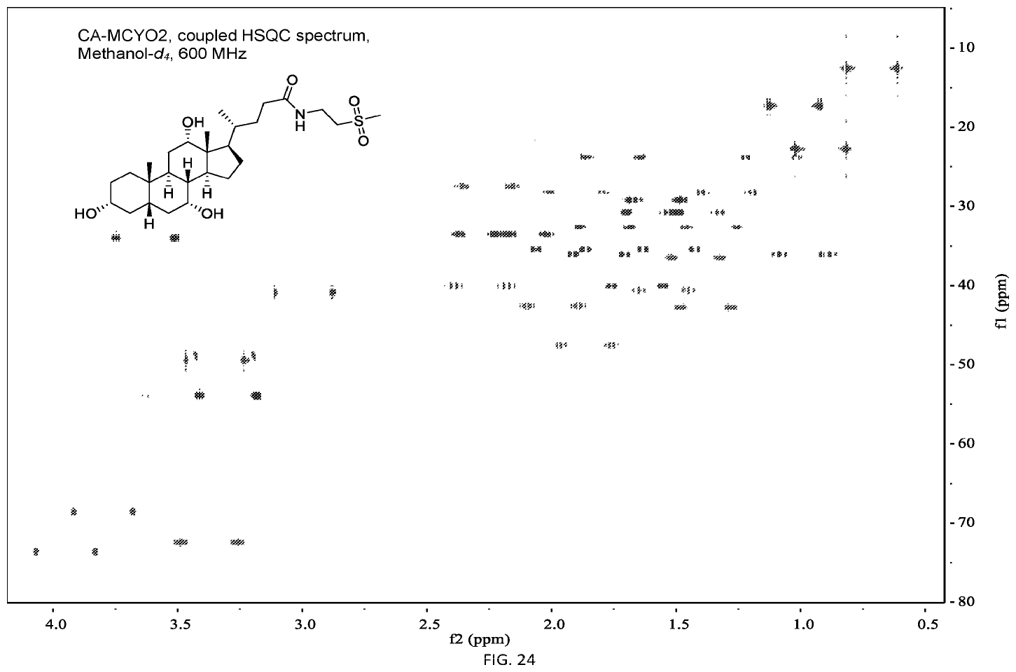

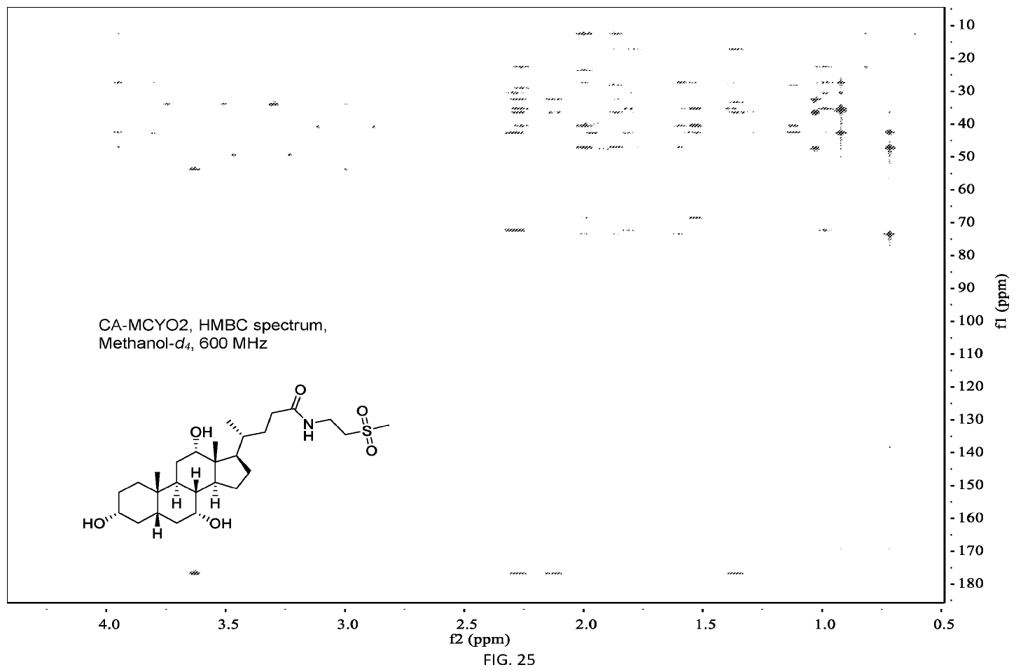

Chemical shifts were referenced to δ(CHD₂D)=3.31 and δ(¹³CHD₂OD)=49.0. ¹³C chemical determined from the acquired ¹H or dqfCOSY spectra. HMBC correlations are from the proton(s) stated to the indicated ¹³C atom. The corresponding ¹H, dqfCOSY, HSQC and HMBC spectra of CA-MCYO2 are shown in FIG. 22 through FIG. 25.

(CA-MCYO2)

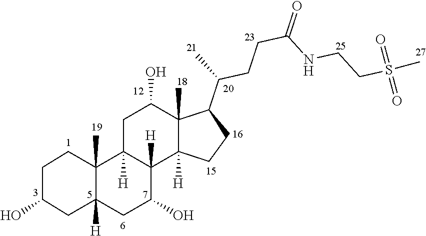

| No. | δc | Proton | δH (J_{HH}[Hz]) | HMBC |
|---|---|---|---|---|
| 1 | 36.2 | 1-Ha | 0.98 (J_{1a,2a} = 3.5 Hz, J_{1a,2b} = 14.0 Hz, J_{1a,1b} = 14.0 Hz) | 19 |
| | | 1-Hb | 1.81 (m) | |
| 2 | 30.9 | 2-Ha | 1.43 (m) | 1a, 4b |
| | | 2-Hb | 1.60 (m) | |
| 3 | 72.6 | 3-H | 3.36 (m) | 1, 4b, 6, 19 |
| 4 | 40.2 | 4-Ha | 1.64 (m) | 6a |
| | | 4-Hb | 2.28 (m) | |
| 5 | 42.8 | 5-H | 1.37 (m) | 1, 4b, 6, 19 |
| 6 | 35.5 | 6-Ha | 1.52 (m) | 4b |
| | | 6-Hb | 1.96 (m) | |
| 7 | 68.6 | 7-1 | 3.80 (m) | 6a, 8 |
| 8 | 40.7 | 8-H | 1.55 (m) | 6a, 7, 9, 11, 14, 15a |
| 9 | 27.6 | 9-H | 2.26 (m) | 1a, 7, 8, 11, 12, 14, |
| | | | | 19 |
| 10 | 35.7 | | | 1, 4b, 5, 6a, 9, 19 |
| 11 | 29.3 | 11-H | 1.58 (m) | 9 |
| 12 | 73.7 | 12-H | 3.95 (m) | 11, 14, 17, 18 |
| 13 | 47.2 | | | 11, 12, 14, 17, 18 |
| 14 | 42.7 | 14-H | 1.99 (m) | 8, 12, 15a, 18 |
| 15 | 23.9 | 15-Ha | 1.12 (J_{15a,16a} = 5.2 Hz, J_{15a,16b} = 11.8 Hz, J_{15a,15b} = | 14 |
| | | | 11.8 Hz, J_{15a,14} = 11.8 Hz) | |
| | | 15-Hb | 1.74 (m) | |
| 16 | 28.4 | 16-Ha | 1.29 (m) | 15a, 17 |
| | | 16-Hb | 1.90 (m) | |
| 17 | 47.6 | 17-H | 1.86 (m) | 18, 21 |
| 18 | 12.7 | 18-H | 0.71 | 12, 14, 17 |
| 19 | 22.9 | 19-H | 0.92 | 1a, 9 |
| 20 | 36.6 | 20-H | 1.41 (m) | 17, 21, 22a |
| 21 | 17.3 | 21-H | 1.03 (J_{21,20} = 6.5 Hz) | 22a |
| 22 | 32.8 | 22-Ha | 1.34 (m) | 21, 23 |
| | | 22-Hb | 1.78 (m) | |
| 23 | 33.7 | 23-Ha | 2.13 (J_{23a,23b} = 13.9 Hz, J_{23a,22a} = 6.6 Hz, J_{23a,22b} = | 22a |
| | | | 10.0 Hz) | |
| | | 23-Hb | 2.26 (m) | |
| 24 | 176.7 | | | 22a, 23, 25 |
| 25 | 34.2 | 25-H | 3.63 (J_{25,26} = 7.0 Hz) | 26 |
| 26 | 54.0 | 26-H | 3.30 (J_{26,25} = 7.0 Hz) | 25, 27 |
| 27 | 41.0 | 27-H | 3.00 | 26 |

### Example 6.7 ¹H (600 MHz) and ¹³C (151 MHz) NMR Spectroscopic Data for CDCA-MCY (Structure Below) in Methanol-d₄

Chemical shifts were referenced to δ(CHD₂OD)=3.31 and δ(¹³CHD₂D)=49.0. ¹³C chemical shifts were determined via 5MBC and HSQC spectra. ¹H, ¹H-J-coupling constants were determined from the acquired ¹H or dqfCOSY spectra. TIM/BC correlations are from the

(CDCA-MCY)

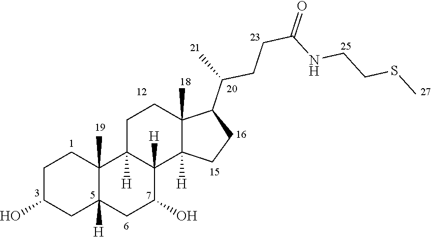

| No. | δc | Proton | δH (J_{HH}[Hz]) | HMBC |
|---|---|---|---|---|
| 1 | 36.3 | 1-Ha | 0.98 (m) | 19 |
| | | 1-Hb | 1.85 (m) | |
| 2 | 31.0 | 2-Ha | 1.33 (m) | 1, 4b |
| | | 2-Hb | 1.60 (m) | |
| 3 | 72.5 | 3-H | 3.36 (m) | 1, 4b |
| 4 | 40.1 | 4-Ha | 1.64 (m) | 5, 6a |
| | | 4-Hb | 2.26 (m) | |
| 5 | 42.8 | 5-H | 1.37 (m) | 1, 4b, 6, 19 |
| 6 | 35.6 | 6-Ha | 1.52 (m) | 4b, 5 |
| | | 6-Hb | 1.95 (m) | |
| 7 | 68.7 | 7-H | 3.78 (m) | 6a, 8 |
| 8 | 40.5 | 8-H | 1.51 (m) | 6a, 7, 9, 11, 14, 15a |
| 9 | 33.8 | 9-H | 1.86 (m) | 1a, 7, 8, 11, 12, |
| | | | | 14, 19 |
| 10 | 33.6 | | | 1, 4b, 5, 6, 9, 19 |
| 11 | 21.5 | 11-Ha | 1.31 (m) | 9 |
| | | 11-Hb | 1.49 (m) | |
| 12 | 40.7 | 12-Ha | 1.20 (m) | 9, 11, 14, 17, 18 |
| | | 12-Hb | 2.00 (m) | |
| 13 | 43.4 | | | 11, 12, 14, 17, 18 |
| 14 | 51.2 | 14-H | 1.49 (m) | 8, 12, 15a, 18 |
| 15 | 24.3 | 15-Ha | 1.09 (m) | 14, 16b |
| | | 15-Hb | 1.73 (m) | |
| 16 | 29.0 | 16-Ha | 1.32 (m) | 15a, 17 |
| | | 16-Hb | 1.91 (m) | |
| 17 | 57.0 | 17-H | 1.17 (m) | 16, 18, 21 |
| 18 | 11.9 | 18-H | 0.70 | 12, 14, 17 |
| 19 | 23.1 | 19-H | 0.93 | 1a, 9 |
| 20 | 36.6 | 20-H | 1.45 (m) | 17, 21, 22a |
| 21 | 18.6 | 21-H | 0.98 (J_{21,20} = 6.6 Hz) | 22 |
| 22 | 32.9 | 22-Ha | 1.29 (m) | 21, 23 |
| | | 22-Hb | 1.80 (m) | |
| 23 | 33.8 | 23-Ha | 2.10 (m) | 22a |
| | | 23-Hb | 2.24 (m) | |
| 24 | 176.4 | | | 22a, 23, 25 |
| 25 | 39.1 | 25-H | 3.35 (J_{25,26} = 7.0 Hz) | 26 |
| 26 | 33.9 | 26-H | 2.58 (J_{26,25} = 7.0 Hz) | 25, 27 |
| 27 | 14.8 | 27-H | 2.10 | 26 |

### Example 6.8 ¹H (600 MHz) and ¹³C (151 MHz) NMR Spectroscopic Data for CDCA-MCYO (Structure Below) in Methanol-d₄

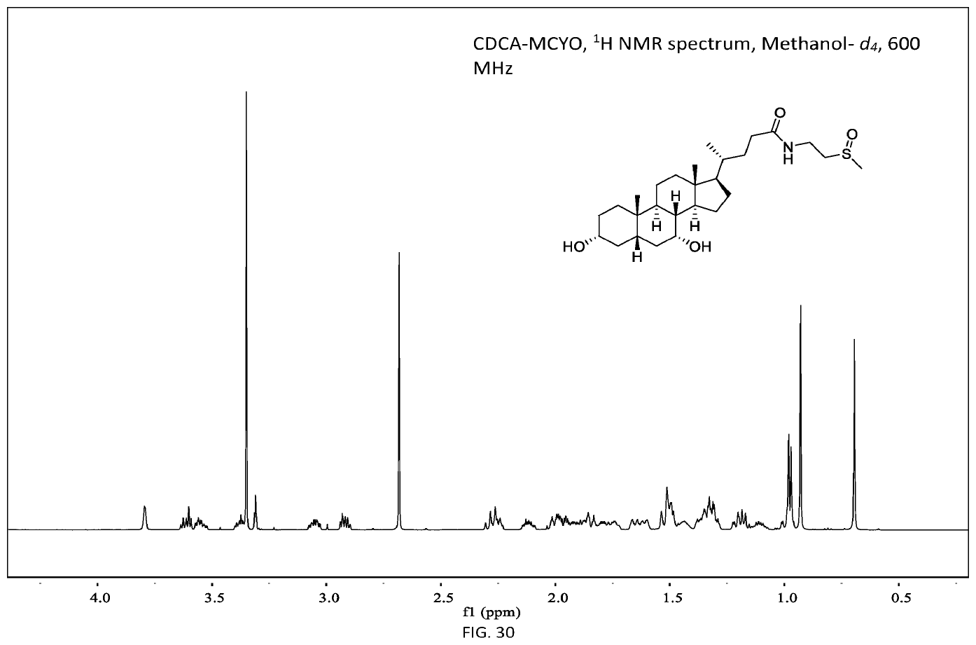

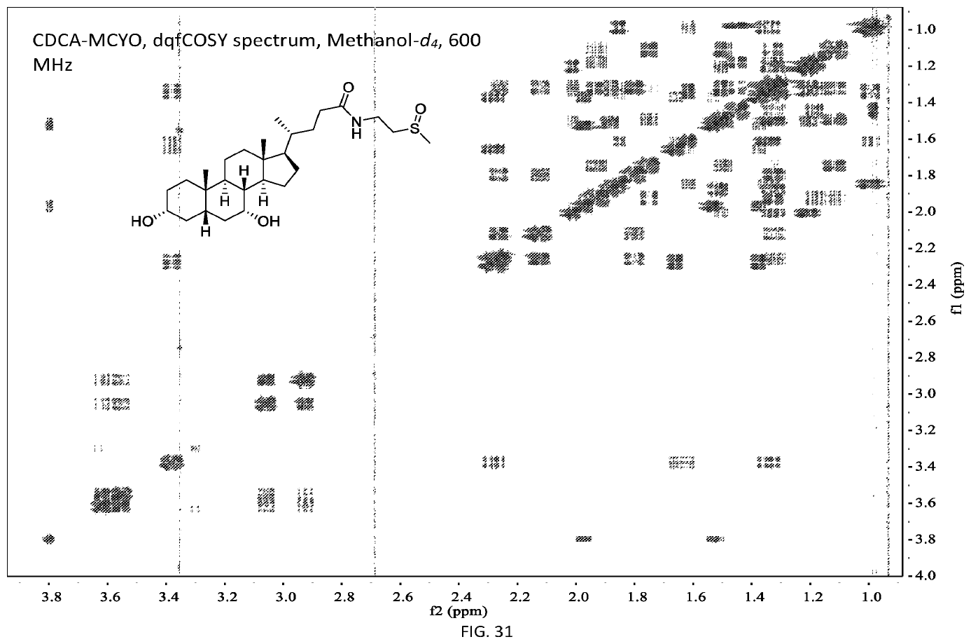

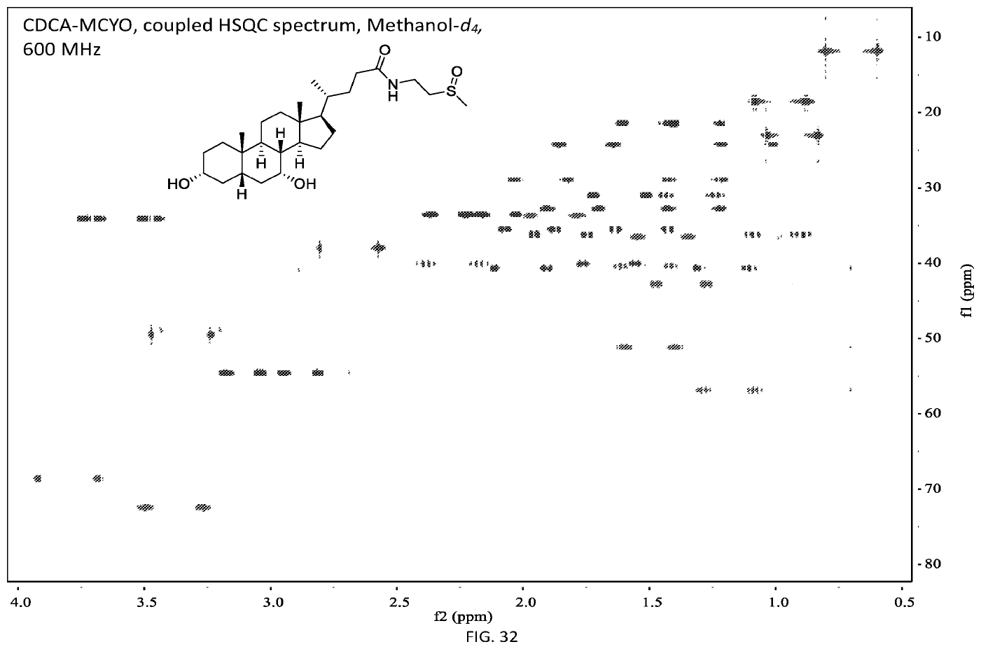

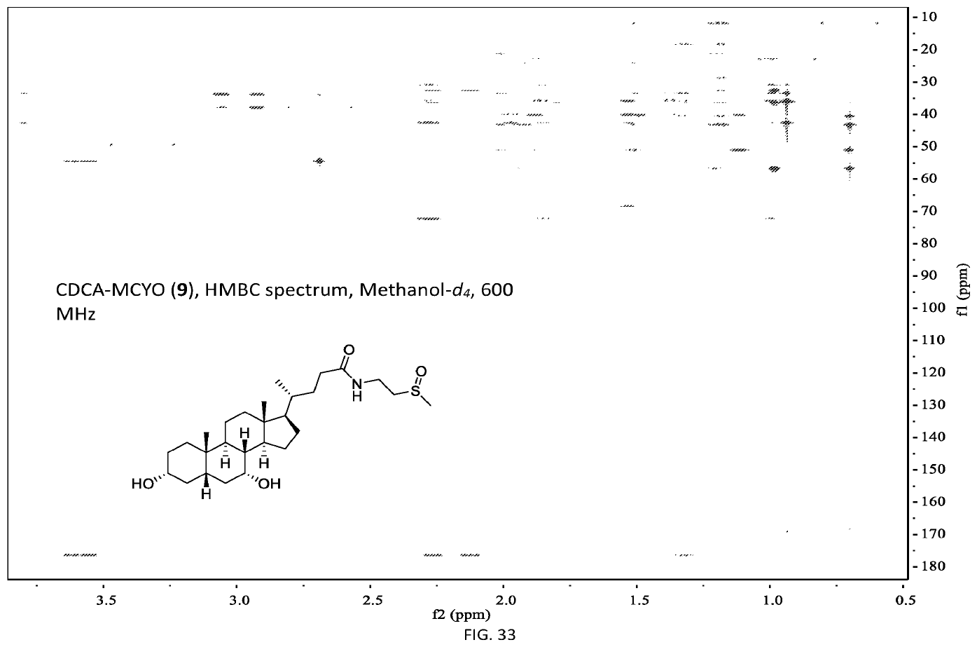

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of CDCA-MCYO are shown in FIG. 30 through FIG. 33.

(CDCA-MCYO)

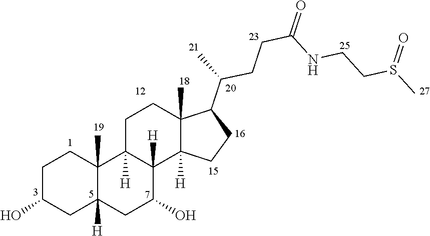

| No. | δc | Proton | δH (J_{HH}[Hz]) | HMBC |
|---|---|---|---|---|
| 1 | 36.3 | 1-Ha | 1.00 (m) | 19 |
| | | 1-Hb | 1.85 (m) | |
| 2 | 31.0 | 2-Ha | 1.33 (m) | 1, 4b |
| | | 2-Hb | 1.60 (m) | |
| 3 | 72.5 | 3-H | 3.36 (m) | 1, 4b |
| 4 | 40.1 | 4-Ha | 1.64 (m) | 5, 6a |
| | | 4-Hb | 2.26 (m) | |
| 5 | 42.8 | 5-H | 1.37 (m) | 1, 4b, 6, 19 |
| 6 | 35.6 | 6-Ha | 1.52 (m) | 4b, 5 |
| | | 6-Hb | 1.95 (m) | |
| 7 | 68.7 | 7-H | 3.78 (m) | 6a, 8 |
| 8 | 40.5 | 8-H | 1.51 (m) | 6a, 7, 9, 11, 14, 15a |
| 9 | 33.8 | 9-H | 1.86 (m) | 1a, 7, 8, 11, 12, 14, 19 |
| 10 | 33.7 | | | 1, 4b, 5, 6, 9, 19 |
| 11 | 21.5 | 11-Ha | 1.31 (m) | 9 |
| | | 11-Hb | 1.49 (m) | |
| 12 | 40.7 | 12-Ha | 1.20 (m) | 9, 11, 14, 17, 18 |
| | | 12-Hb | 2.00 (m) | |
| 13 | 43.4 | | | 11, 12, 14, 17, 18 |
| 14 | 51.2 | 14-H | 1.49 (m) | 8, 12, 15a, 18 |
| 15 | 24.3 | 15-Ha | 1.11 (m) | 14, 16b |
| | | 15-Hb | 1.74 (m) | |
| 16 | 29.0 | 16-Ha | 1.32 (m) | 15a, 17 |
| | | 16-Hb | 1.93 (m) | |
| 17 | 57.0 | 17-H | 1.17 (m) | 16, 18, 21 |
| 18 | 11.9 | 18-H | 0.69 | 12, 14, 17 |
| 19 | 23.1 | 19-H | 0.93 | 1a, 9 |
| 20 | 36.6 | 20-H | 1.44 (m) | 17, 21, 22a |
| 21 | 18.6 | 21-H | 0.98 (J_{21,20} = 6.6 Hz) | 22 |
| 22 | 32.9 | 22-Ha | 1.31 (m) | 21, 23 |
| | | 22-Hb | 1.80 (m) | |
| 23 | 33.6 | 23-Ha | 2.10 (m) | 22a |
| | | 23-Hb | 2.24 (m) | |
| 24 | 176.7 | | | 22a, 23, 25 |
| 25 | 34.2 | 25-Ha | 3.55 (m) | 26 |
| | | 25-Hb | 3.61 (J_{25b,25a} = 12.5 Hz, J_{25b,26a} = 6.0 Hz, J_{25a,26b} = 6.5 Hz) | 25, 27 |
| 26 | 54.7 | 26-Ha | 2.92 (J_{26a,26b} = 12.0 Hz, J_{26a,26a} = 6.0 Hz, J_{26a,26b} = 6.5 Hz) | 26 |
| | | 26-Hb | 3.05 (m) | 25, 27 |
| 27 | 38.2 | 27-H | 2.68 | 26 |

### Example 6.9 ¹H (600 MHz) and ¹³C (151 MHz) NMR Spectroscopic Data for UDCA-MCY (Structure Below) in Methanol-d₄

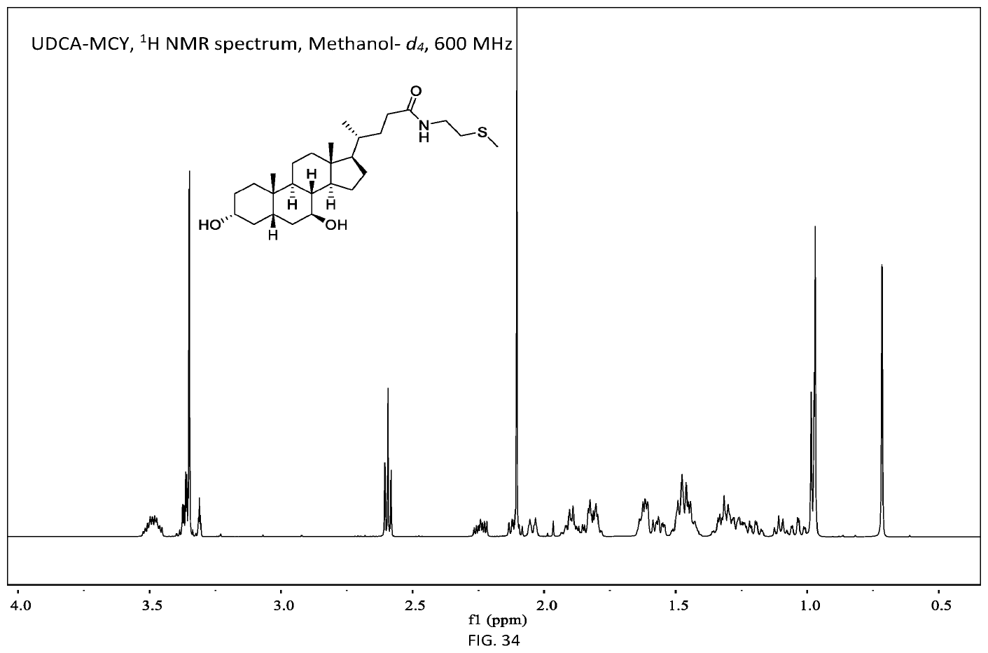

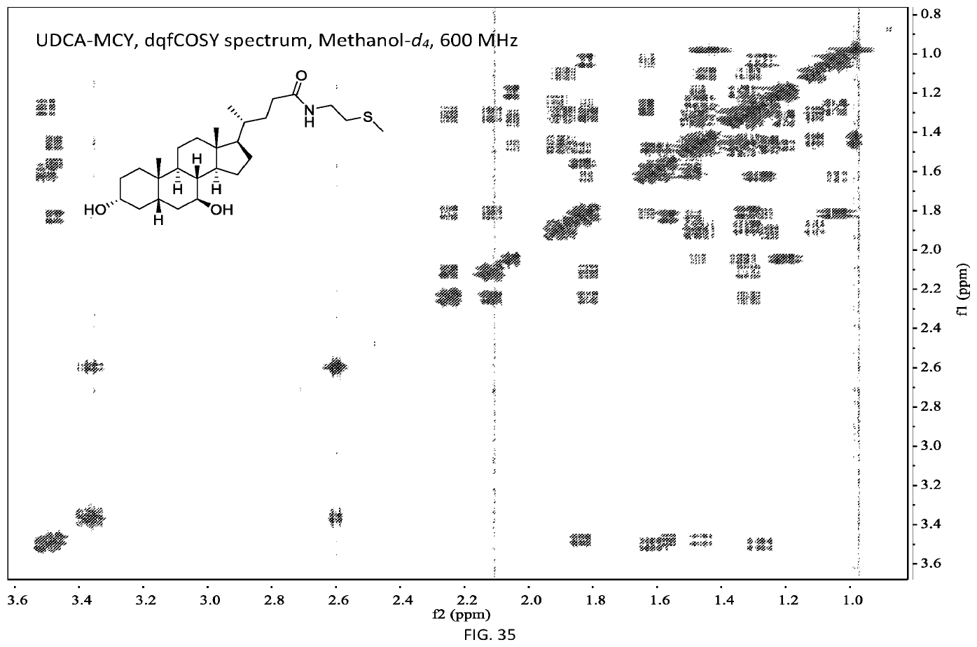

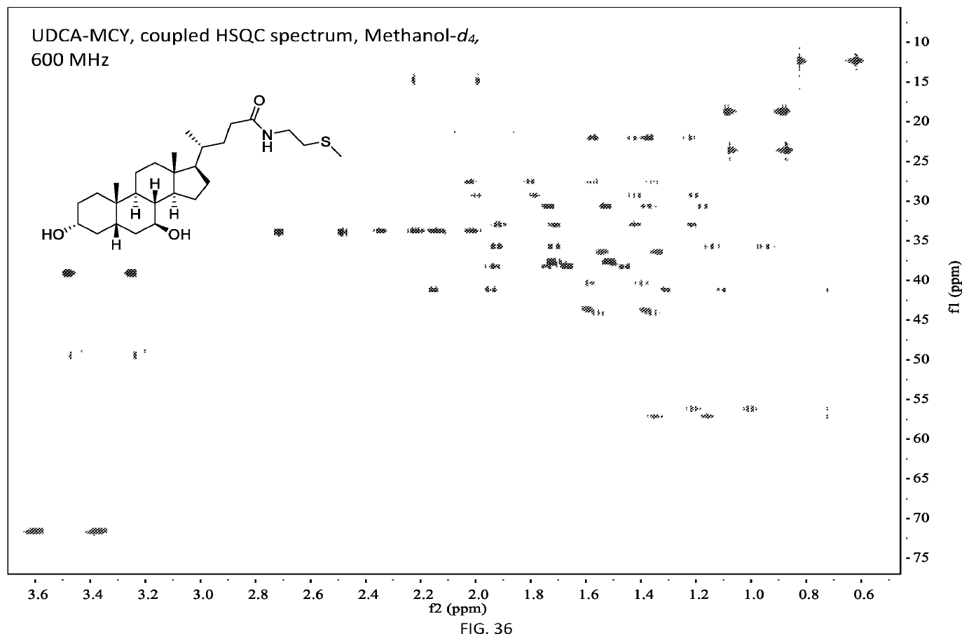

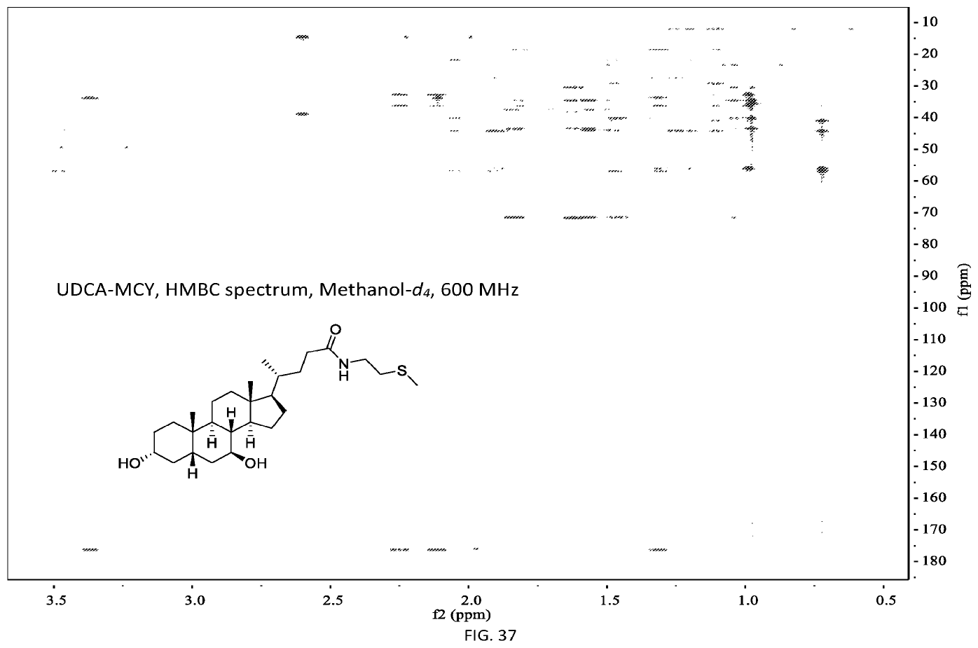

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of UDCA-MCY are shown in FIG. 34 through FIG. 37.

(UDCA-MCY)

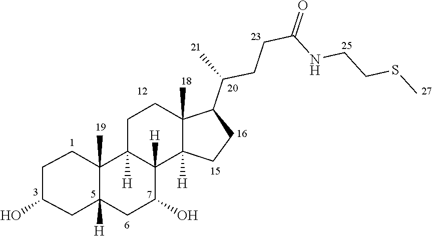

| No. | δc | Proton | δH (J_{HH}[Hz]) | HMBC |
|---|---|---|---|---|
| 1 | 35.8 | 1-Ha | 1.03 (m) | 2, 19 |
| | | 1-Hb | 1.82 (m) | |
| 2 | 30.7 | 2-Ha | 1.28 (m) | 1, 4 |
| | | 2-Hb | 1.64 (m) | |
| 3 | 71.6 | 3-H | 3.47 (m) | 1, 2b, 4 |
| 4 | 37.8 | 4-H | 1.62 (m) | 5, 6a |
| 5 | 43.8 | 5-H | 1.47 (m) | 1, 4, 6, 19 |
| 6 | 38.4 | 6-Ha | 1.57 (m) | 4,5 |
| | | 6-Hb | 1.83 (m) | |
| 7 | 71.8 | 7-H | 3.50 (m) | 6, 8 |
| 8 | 44.2 | 8-H | 1.45 (m) | 6a, 14, 15a |
| 9 | 40.5 | 9-H | 1.51 (m) | 1a, 8, 11, 12a, 19 |
| 10 | 34.9 | | | 1, 4, 5, 6, 9, 19 |
| 11 | 22.1 | 11-Ha | 1.31 (m) | 12 |
| | | 11-Hb | 1.47 (m) | |
| 12 | 41.3 | 12-Ha | 1.20 (m) | 11b, 17, 18 |
| | | 12-Hb | 2.04 (m) | |
| 13 | 44.5 | | | 12, 14, 17, 18 |
| 14 | 57.3 | 14-H | 1.25 (m) | 8, 12a, 15a, 18 |
| 15 | 27.6 | 15-Ha | 1.46 (m) | 14, 16b |
| | | 15-Hb | 1.91 (m) | |
| 16 | 29.4 | 16-Ha | 1.31 (m) | 15a, 17 |
| | | 16-Hb | 1.90 (m) | |
| 17 | 56.3 | 17-H | 1.10 (m) | 16, 18, 21 |
| 18 | 12.4 | 18-H | 0.72 | 14, 17 |
| 19 | 23.7 | 19-H | 0.97 | 1a |
| 20 | 36.5 | 20-H | 1.44 (m) | 17, 21, 22a, 23 |
| 21 | 18.7 | 21-H | 0.98 (J_{21,20} = 6.6 Hz) | 22 |
| 22 | 33.1 | 22-Ha | 1.31 (m) | 21, 23 |
| | | 22-Hb | 1.81 (m) | |
| 23 | 33.9 | 23-Ha | 2.11 (m) | 22a |
| | | 23-Hb | 2.24 (m) | |
| 24 | 176.4 | | | 22a, 23, 25 |
| 25 | 39.2 | 25-H | 3.36 (J_{25,26} = 7.0 Hz) | 26 |
| 26 | 34.0 | 26-H | 2.59 (J_{26,25} = 7.0 Hz) | 25, 27 |
| 27 | 14.9 | 27-H | 2.10 | 26 |

### Example 6.10, ¹H (600 MHz) and ¹³C (151 MHz) NMR Spectroscopic Data for UDCA-MCYO (Structure Below) in Methanol-d₄

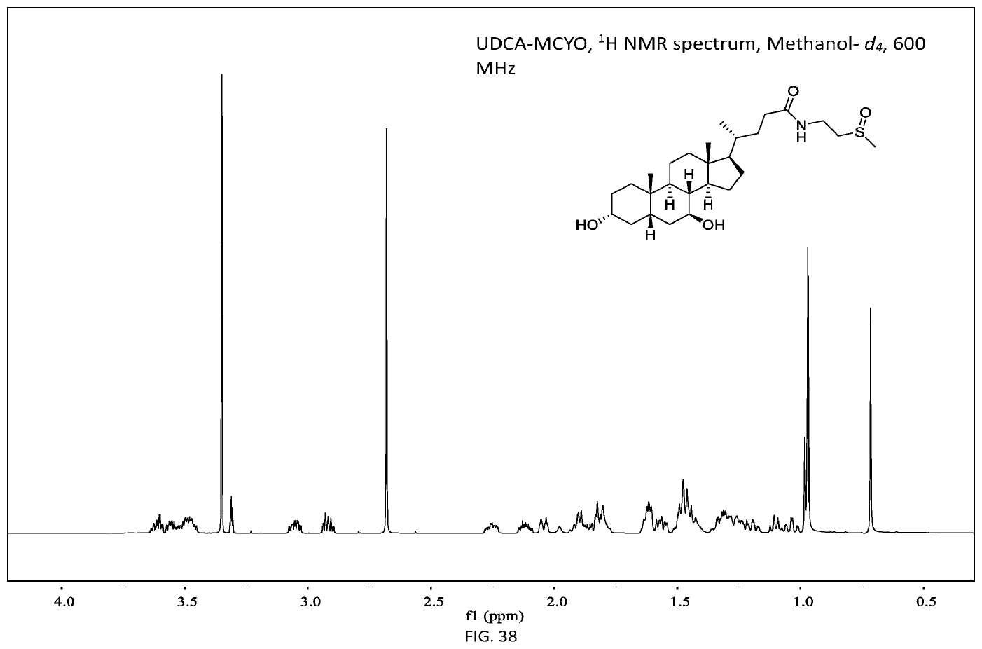

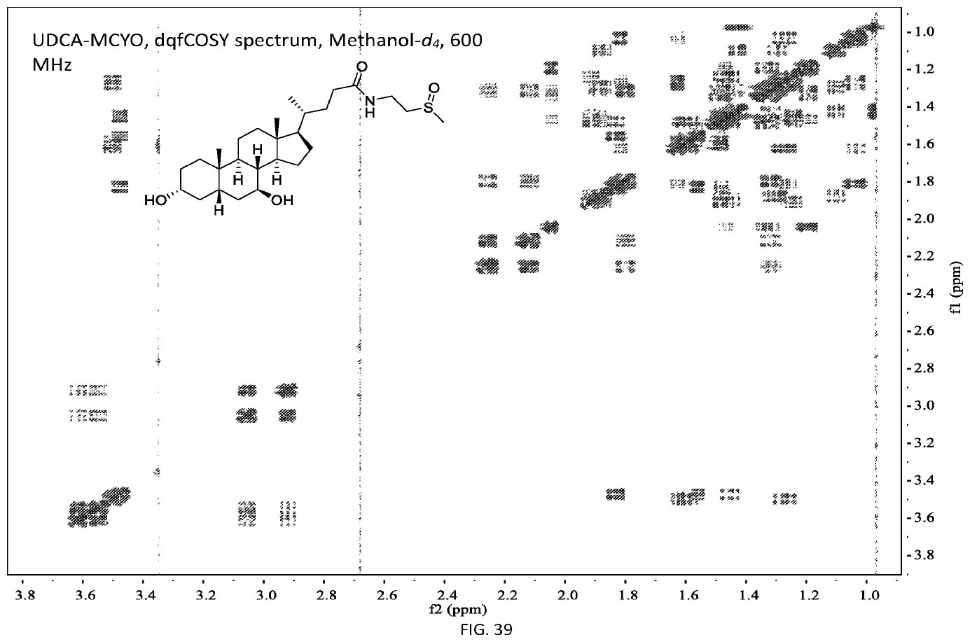

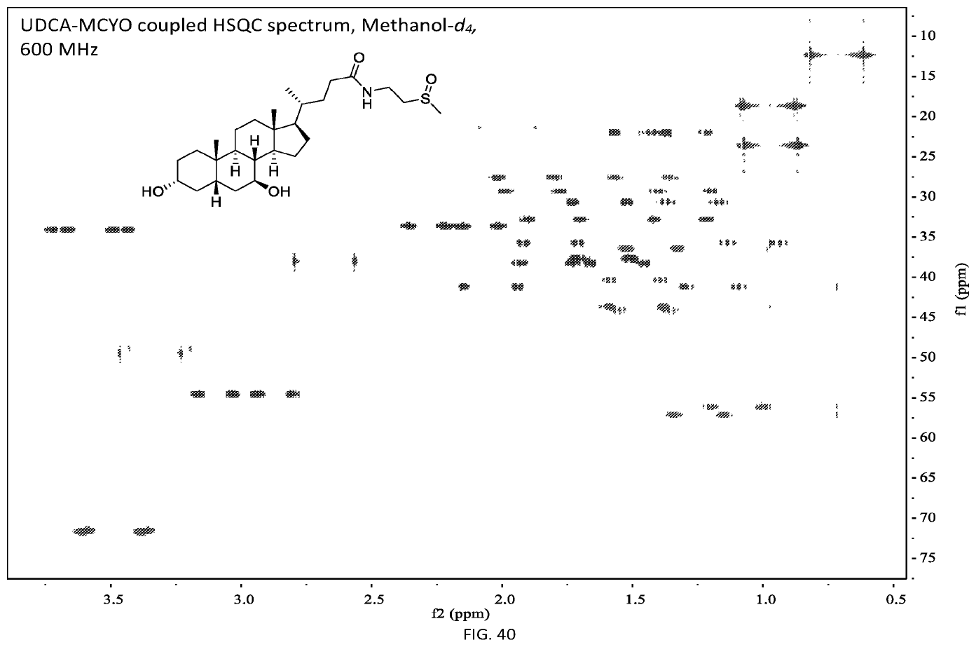

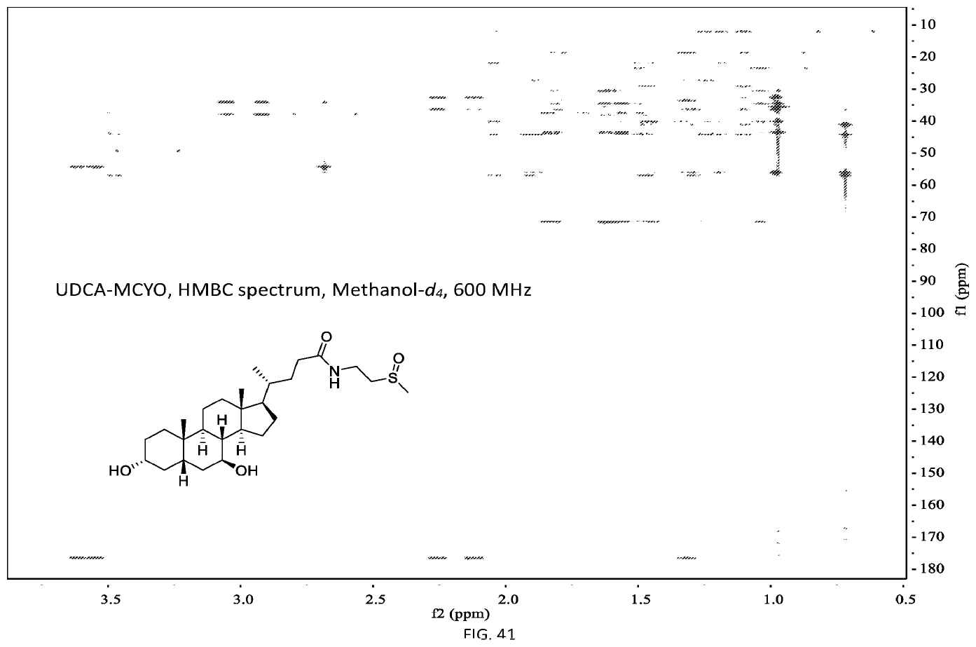

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of UDCA-MCYO are shown in FIG. 38 through FIG. 41.

(UDCA-MCYO)

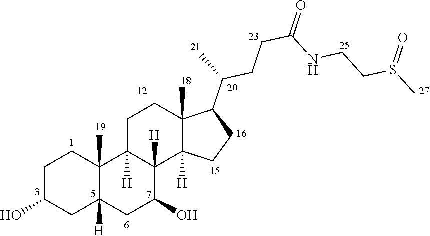

| No. | δc | Proton | δH (J_{HH}[Hz]) | HMBC |
|---|---|---|---|---|
| 1 | 35.8 | 1-Ha | 1.03 (m) | 2, 19 |
| | | 1-Hb | 1.82 (m) | |
| 2 | 30.7 | 2-Ha | 1.27 (m) | 1, 4 |
| | | 2-Hb | 1.62 (m) | |
| 3 | 71.6 | 3-H | 3.50 (m) | 1, 2b, 4 |
| 4 | 37.8 | 4-H | 1.62 (m) | 5, 6a |
| 5 | 43.8 | 5-H | 1.47 (m) | 1, 4, 6, 19 |
| 6 | 38.4 | 6-Ha | 1.57 (m) | 4, 5 |
| | | 6-Hb | 1.83 (m) | |
| 7 | 71.8 | 7-H | 3.50 (m) | 6, 8 |
| 8 | 44.2 | 8-H | 1.45 (m) | 6a, 14, 15a |
| 9 | 40.5 | 9-H | 1.49 (m) | 1a, 8, 11, 12a, 19 |
| 10 | 34.9 | | | 1, 4, 5, 6, 9, 19 |
| 11 | 22.1 | 11-Ha | 1.31 (m) | 12 |
| | | 11-Hb | 1.47 (m) | |
| 12 | 41.3 | 12-Ha | 1.20 (m) | 11b, 17, 18 |
| | | 12-Hb | 2.04 (m) | |
| 13 | 44.5 | | | 12, 14, 17, 18 |
| 14 | 57.3 | 14-H | 1.24 (m) | 8, 12a, 15a, 18 |
| 15 | 27.6 | 15-Ha | 1.46 (m) | 14, 16b |
| | | 15-Hb | 1.91 (m) | |
| 16 | 29.4 | 16-Ha | 1.31 (m) | 15a, 17 |
| | | 16-Hb | 1.90 (m) | |
| 17 | 56.3 | 17-H | 1.10 (m) | 16, 18, 21 |
| 18 | 12.4 | 18-H | 0.72 | 14, 17 |
| 19 | 23.7 | 19-H | 0.97 | 1a |
| 20 | 36.5 | 20-H | 1.43 (m) | 17, 21, 22a, 23 |
| 21 | 18.7 | 21-H | 0.98 (J_{21,20} = 6.5 Hz) | 22 |
| 22 | 33.1 | 22-Ha | 1.31 (m) | 21, 23 |
| | | 22-Hb | 1.80 (m) | |
| 23 | 33.9 | 23-Ha | 2.11 (m) | 22a |
| | | 23-Hb | 2.24 (m) | |
| 24 | 176.6 | | | 22a, 23, 25 |
| 25 | 34.2 | 25-H | 3.36 (J_{25,26} = 7.0 Hz) | 26 |
| 26 | 54.7 | 26-Ha | 2.92 (m) | 25, 27 |
| | | 26-Hb | 3.05 (m) | 26 |
| 27 | 38.1 | 27-H | 2.68 | 26 |

### Example 6.11, ¹H (600 MHz) and ¹³C (151 MHz) NMR Spectroscopic Data for DCA-MCY (Structure Below) in Methanol-d₄

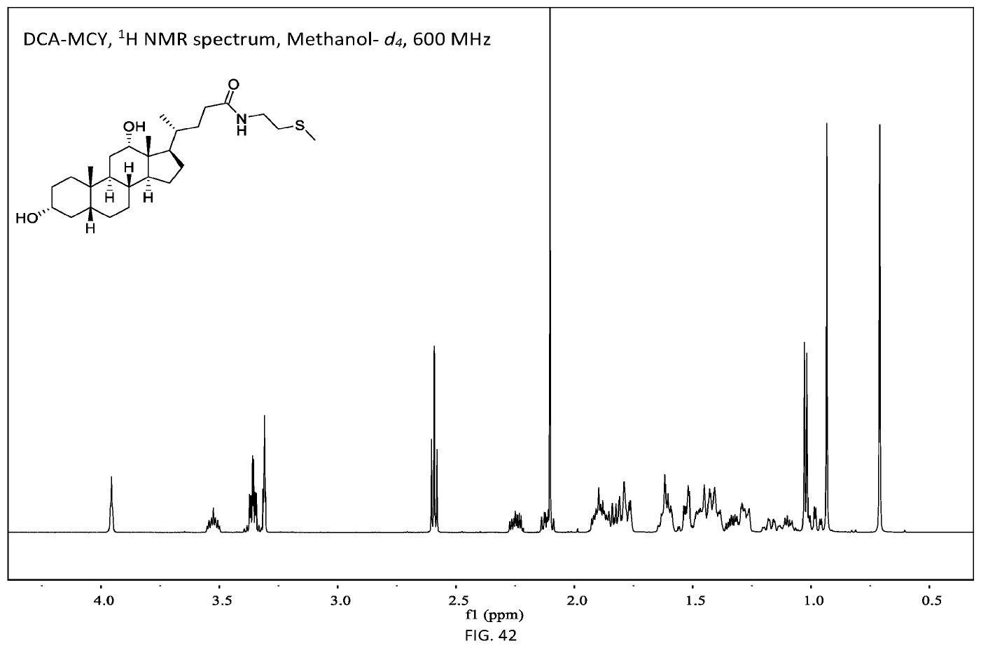

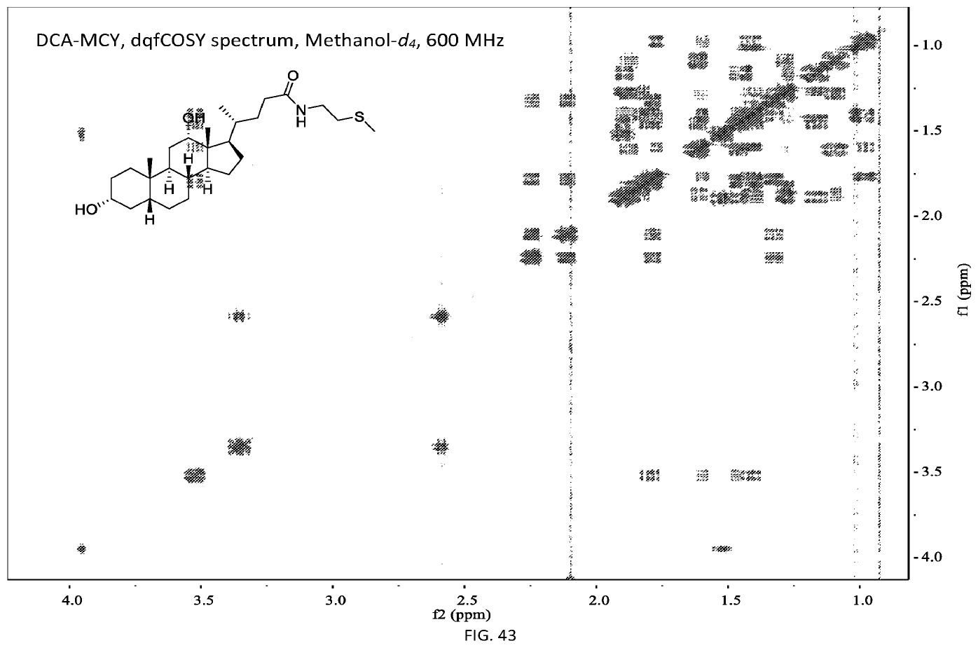

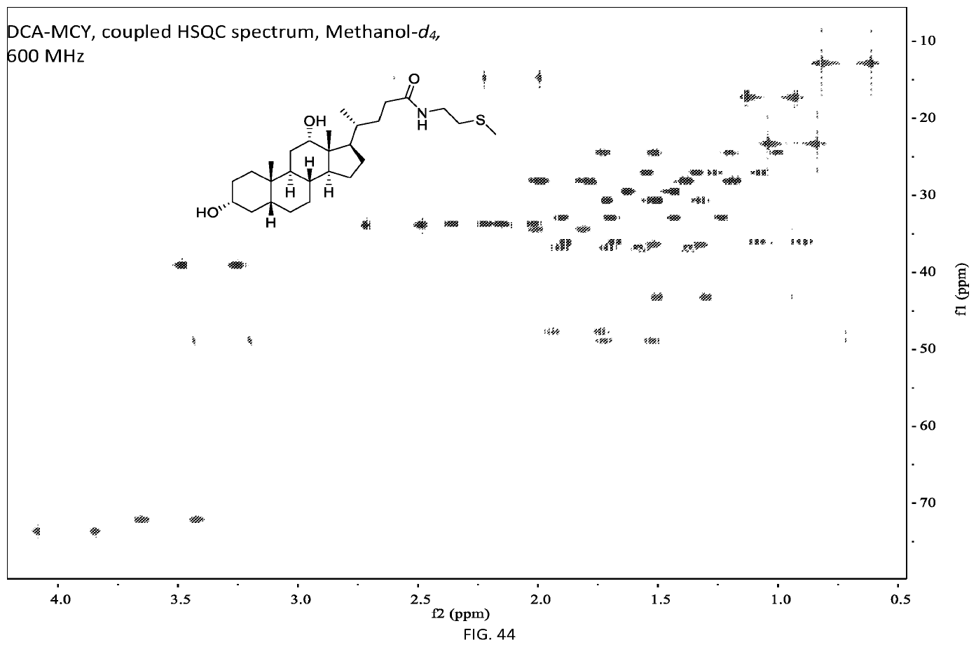

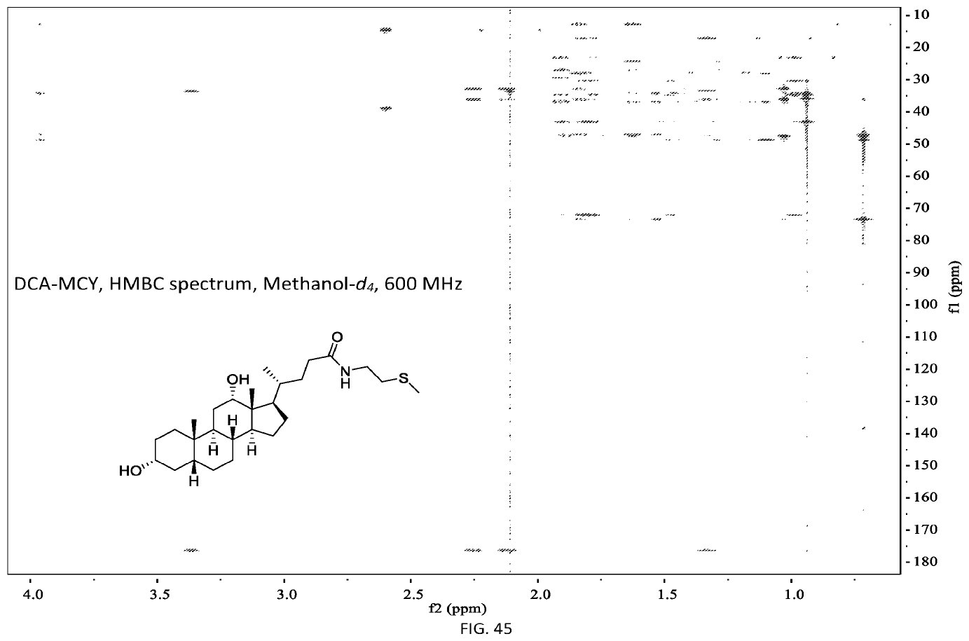

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of DCA-MCY are shown in FIG. 42 through FIG. 45.

(DCA-MCY)

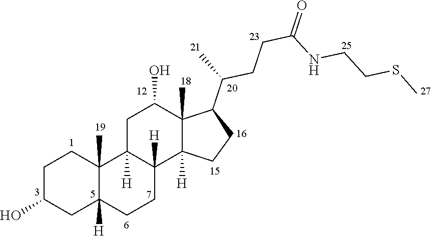

| No. | δc | Proton | δH (J_{HH}[Hz]) | HMBC |
|---|---|---|---|---|
| 1 | 36.1 | 1-Ha | 0.98 (J_{1a,2a} = 3.5 Hz, | 19 |
| | | | J_{1a,2b} = 14.0 Hz, | |
| | | | J_{1a,1b} = 14.0 Hz) | |
| | | 1-Hb | 1.78 (m) | |
| 2 | 30.8 | 2-Ha | 1.43 (m) | 1a, 4a |
| | | 2-Hb | 1.60 (m) | |
| 3 | 72.2 | 3-H | 3.53 (m) | 1, 4a |
| 4 | 36.9 | 4-Ha | 1.48 (m) | 6b |
| | | 4-Hb | 1.80 (m) | |
| 5 | 43.4 | 5-H | 1.40 (m) | 1b, 4a, 6b, 19 |
| 6 | 28.3 | 6-Ha | 1.29 (m) | 4a, 7a |
| | | 6-Hb | 1.88 (m) | |
| 7 | 27.1 | 7-Ha | 1.17 (m) | 6, 8 |
| | | 7-Hb | 1.44 (m) | |
| 8 | 37.4 | 8-H | 1.47 (m) | 6b, 9, 11, 14, 15a |
| 9 | 34.5 | 9-H | 1.91 (m) | 1a, 8, 11, 12, 19 |
| 10 | 34.8 | | | 1, 4b, 5, 6b, 9, 19 |
| 11 | 29.6 | 11-H | 1.52 (m) | 9 |
| 12 | 73.7 | 12-H | 3.96 (m) | 11, 17, 18 |
| 13 | 47.3 | | | 11, 12, 14, 17, 18 |
| 14 | 49.0 | 14-H | 1.62 (m) | 8, 12, 15a, 18 |
| 15 | 24.6 | 15-Ha | 1.10 (m) | 14 |
| | | 15-Hb | 1.62 (m) | |
| 16 | 28.2 | 16-Ha | 1.28 (m) | 15a, 17 |
| | | 16-Hb | 1.89 (m) | |
| 17 | 47.8 | 17-H | 1.84 (m) | 18, 21 |
| 18 | 12.9 | 18-H | 0.72 | 12, 14, 17 |
| 19 | 23.4 | 19-H | 0.93 | 1a, 9 |
| 20 | 36.6 | 20-H | 1.41 (m) | 17, 21, 22a |
| 21 | 17.4 | 21-H | 1.02 (J_{21,20} = 6.6 Hz) | 22a |
| 22 | 32.9 | 22-Ha | 1.33 (m) | 21, 23 |
| | | 22-Hb | 1.80 (m) | |
| 23 | 33.8 | 23-Ha | 2.12 (m) | 22a |
| | | 23-Hb | 2.25 (m) | |
| 24 | 176.5 | | | 22a, 23, 25 |
| 25 | 39.1 | 25-H | 3.37 (J_{25,26} = 7.0 Hz) | 26 |
| 26 | 34.0 | 26-H | 2.59 (J_{26,25} = 7.0 Hz) | 25, 27 |
| 27 | 14.9 | 27-H | 2.10 | 26 |

### Example 6.12, H (600 MHz) and ¹³C (151 MHz) NMR Spectroscopic Data for DCA-MCYO (Structure Below) in Methanol-d₄

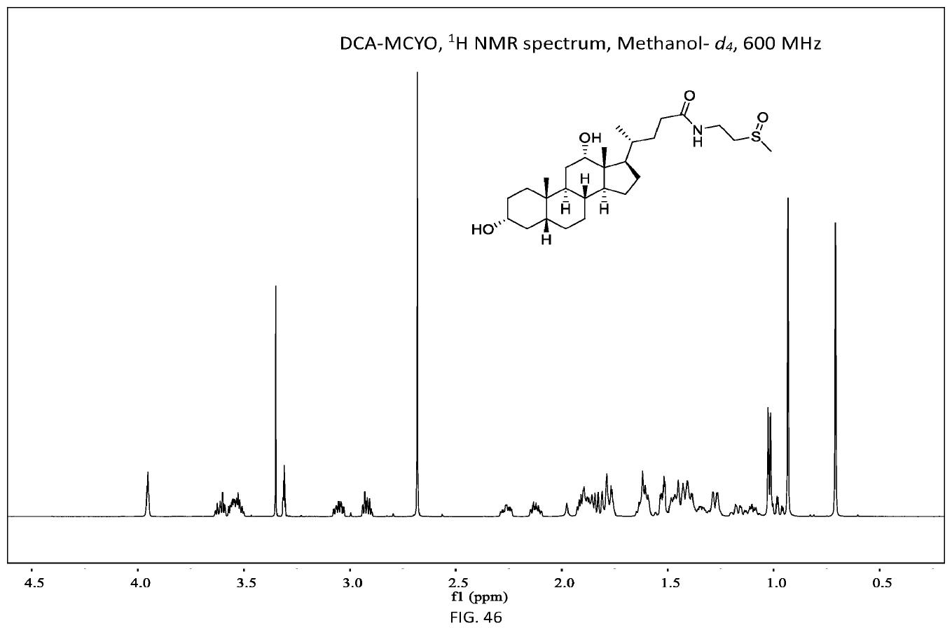

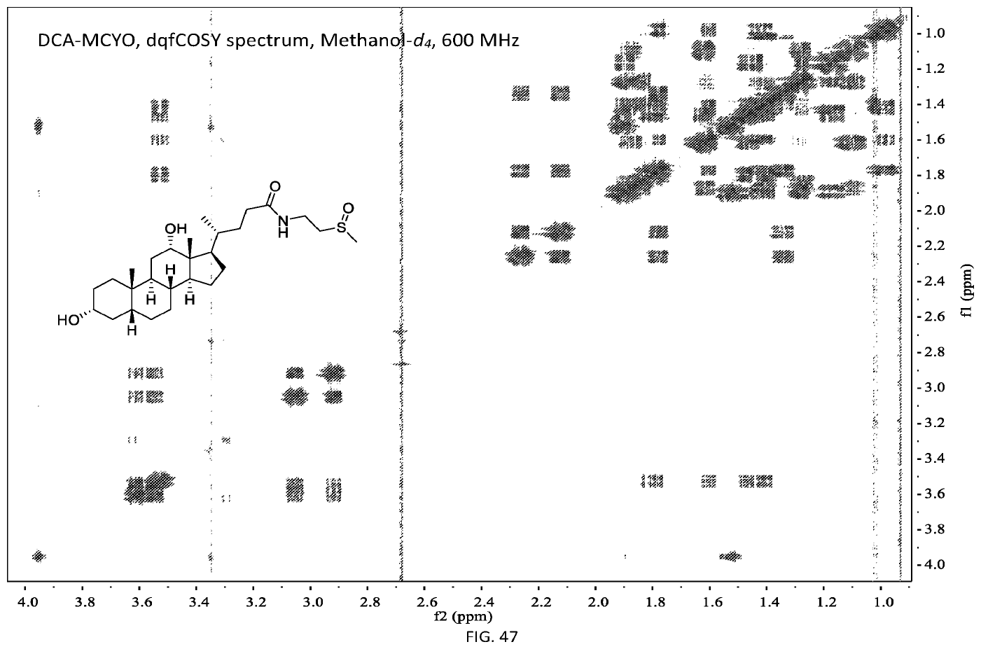

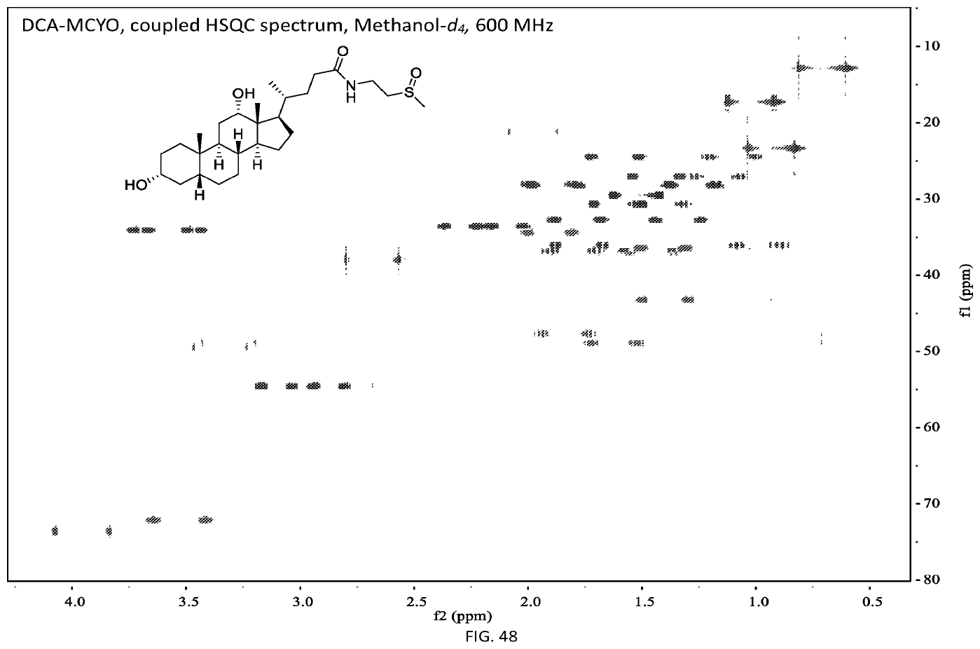

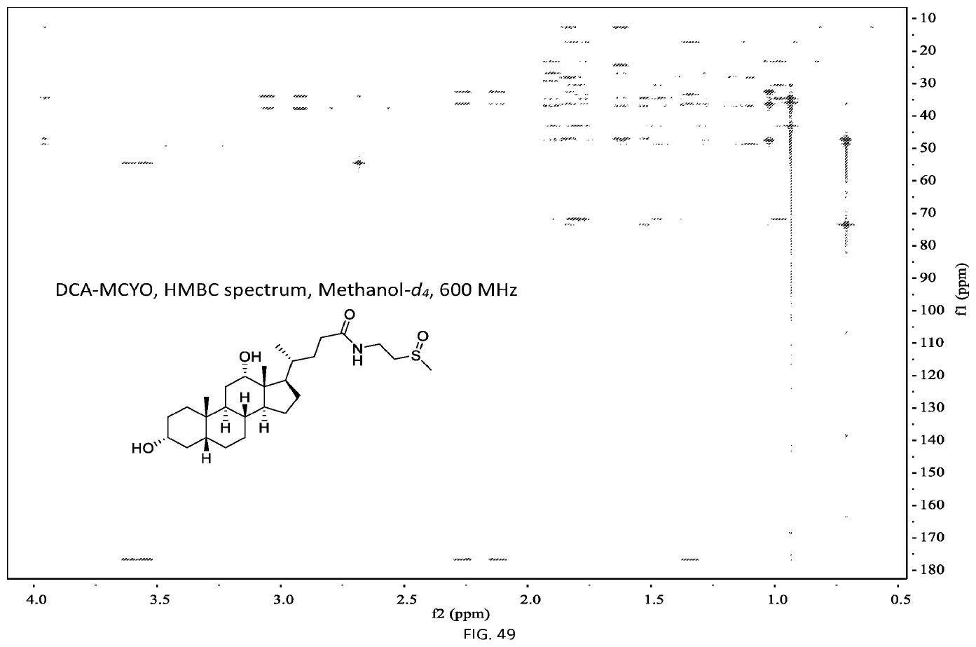

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of KDCA-MCY are shown in FIG. 46 through FIG. 49.

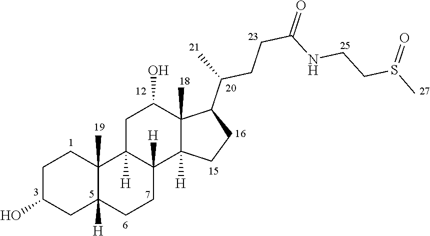

| No. | δc | Proton | δH (J_{HH}[Hz]) | HMBC |
|---|---|---|---|---|
| 1 | 36.1 | 1-Ha | 0.98 (m) | 19 |
| | | 1-Hb | 1.78 (m) | |
| 2 | 30.8 | 2-Ha | 1.43 (m) | 1a, 4a |
| | | 2-Hb | 1.60 (m) | |
| 3 | 72.2 | 3-H | 3.53 (m) | 1, 4a |
| 4 | 36.9 | 4-Ha | 1.48 (m) | 6b |
| | | 4-Hb | 1.80 (m) | |
| 5 | 43.4 | 5-1 | 1.40 (m) | 1b, 4a, 6b, 19 |
| 6 | 28.3 | 6-Ha | 1.29 (m) | 4a, 7a |
| | | 6-Hb | 1.88 (m) | |
| 7 | 27.1 | 7-Ha | 1.17 (m) | 6, 8 |
| | | 7-Hb | 1.44 (m) | |
| 8 | 37.3 | 8-H | 1.46 (m) | 6b, 9, 11, 14, 15a |
| 9 | 34.5 | 9-H | 1.90 (m) | 1a, 8, 11, 12, 19 |
| 10 | 34.8 | | | 1, 4b, 5, 6b, 9, 19 |
| 11 | 29.6 | 11-H | 1.52 (m) | 9 |
| 12 | 73.7 | 12-H | 3.95 (m) | 11, 17, 18 |
| 13 | 47.3 | | | 11, 12, 14, 17, 18 |
| 14 | 49.0 | 14-H | 1.62 (m) | 8, 12, 15a, 18 |
| 15 | 24.6 | 15-Ha | 1.10 (m) | 14 |
| | | 15-Hb | 1.62 (m) | |
| 16 | 28.2 | 16-Ha | 1.28 (m) | 15a, 17 |
| | | 16-Hb | 1.89 (m) | |
| 17 | 47.8 | 17-H | 1.84 (m) | 18, 21 |
| 18 | 12.9 | 18-H | 0.71 | 12, 14, 17 |
| 19 | 23.4 | 19-H | 0.93 | 1a, 9 |
| 20 | 36.6 | 20-H | 1.41 (m) | 17, 21, 22a |
| 21 | 17.4 | 21-H | 1.02 (J_{21,20} = 6.5 Hz) | 22a |
| 22 | 32.9 | 22-Ha | 1.33 (m) | 21, 23 |
| | | 22-Hb | 1.78 (m) | |
| 23 | 33.7 | 23-Ha | 2.12 (m) | 22a |
| | | 23-Hb | 2.25 (m) | |
| 24 | 176.7 | | | 22a, 23, 25 |
| 25 | 34.2 | 25-Ha | 3.53 (m) | 26 |
| | | 25-Hb | 3.61 (m) | 25, 27 |
| 26 | 54.7 | 26-Ha | 2.92 (m) | 26 |
| | | 26-Hb | 3.05 (m) | 25, 27 |
| 27 | 38.1 | 27-H | 2.68 | 26 |

### Example 6.13 ¹H (600 MHz) and ¹³C (151 MHz) NMR Spectroscopic Data for KDCA-MCY (Structure Below) in Methanol-d₄

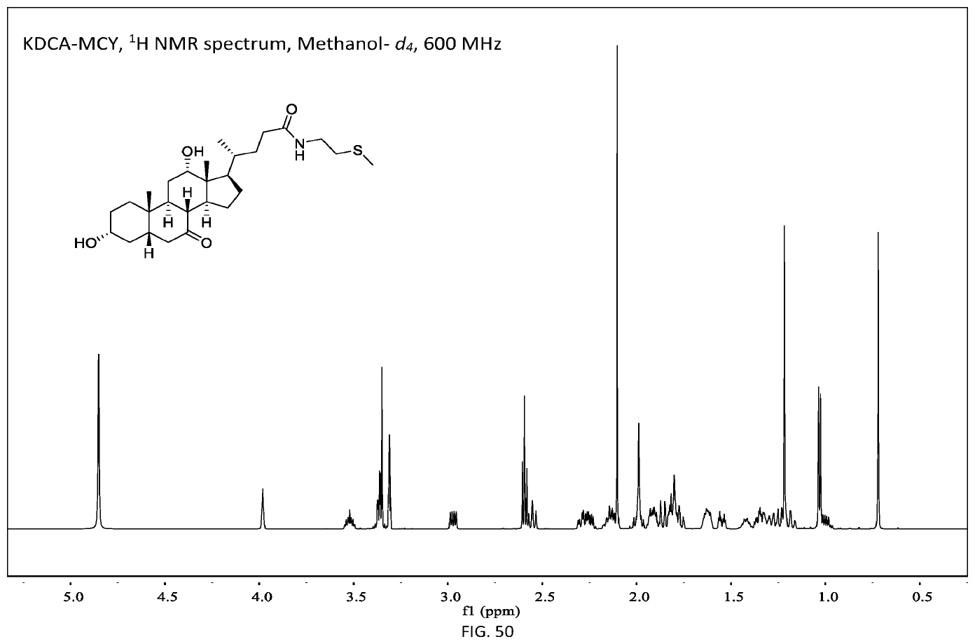

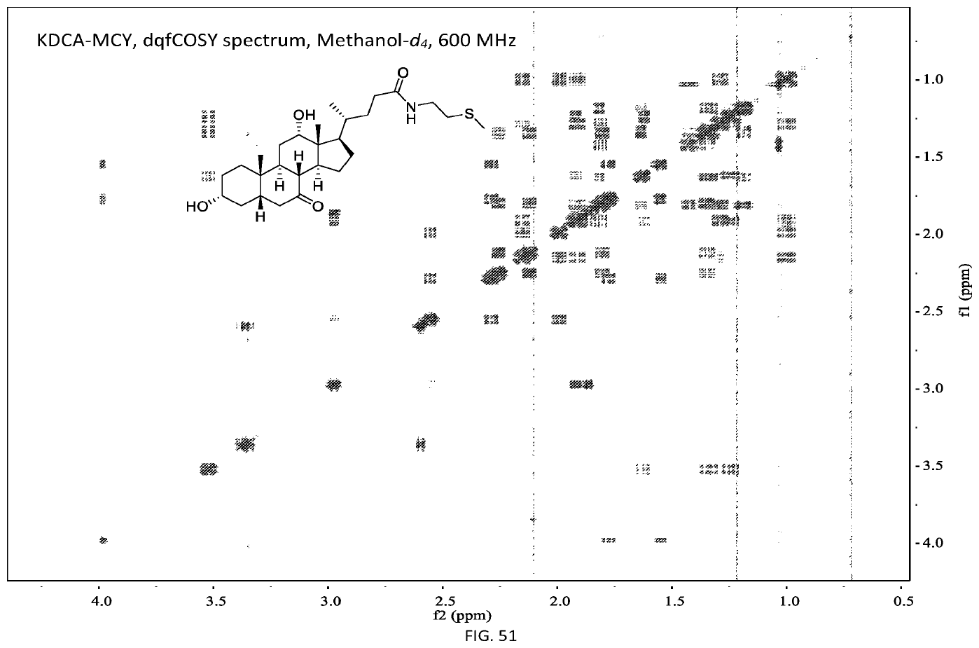

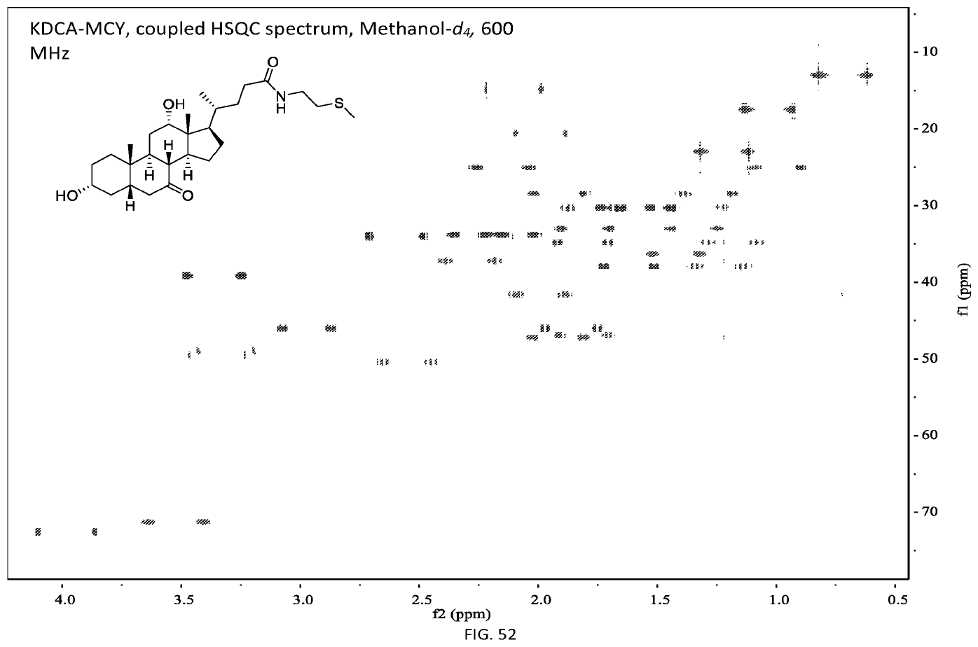

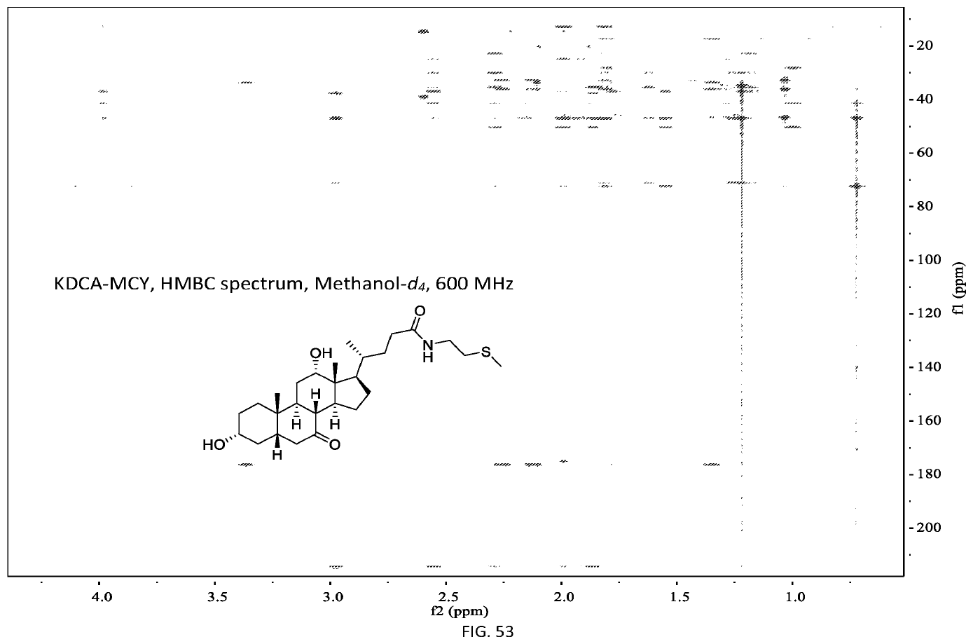

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of KDCA-MCY are shown in FIG. 50 through FIG. 53.

(KDCA-MCY)

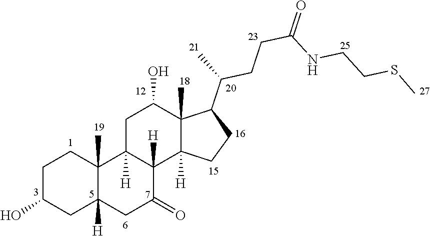

| No. | δc | Proton | δH (J_{HH}[Hz]) | HMBC |
|---|---|---|---|---|
| 1 | 34.8 | 1-Ha | 1.21 (m) | 19 |
| | | 1-Hb | 1.81 (m) | |
| 2 | 30.9 | 2-Ha | 1.33 (m) | |
| | | 2-Hb | 1.64 (m) | |
| 3 | 71.3 | 3-H | 3.52 (m) | 1, 4 |
| 4 | 37.9 | 4-Ha | 1.24 (m) | 6 |
| | | 4-Hb | 1.62 (m) | |
| 5 | 47.2 | 5-H | 1.92 (m) | 1b, 6, 19 |
| 6 | 46.1 | 6-Ha | 1.86 (J_{6a,6b} = 12.7 Hz, | 4a |
| | | | J_{6a,5} = 2.0 Hz) | |
| | | 6-Hb | 2.97 (J_{6b,6a} = 12.7 Hz, | |
| | | | J_{6b,5} = 5.9 Hz) | |
| 7 | 214.5 | | | 6, 8 |
| 8 | 50.4 | 8-H | 2.55 (m) | 6a, 9, 11a, 14, 15a |
| 9 | 37.2 | 9-H | 2.29 (m) | 1a, 8, 11, 12, 19 |
| 10 | 35.6 | | | 1, 4b, 6a, 9, 19 |
| 11 | 30.3 | 11-Ha | 1.55 (m) | 9 |
| | | 11-Hb | 1.78 (m) | |
| 12 | 72.6 | 12-H | 3.99 (m) | 11a, 17, 18 |
| 13 | 47.2 | | | 11, 12, 14, 17, 18 |
| 14 | 41.6 | 14-H | 1.99 (m) | 8, 12, 15a, 18 |
| 15 | 25.1 | 15-Ha | 1.00 (m) | 14 |
| | | 15-Hb | 2.15 (m) | |
| 16 | 28.5 | 16-Ha | 1.29 (m) | 15a, 17 |
| | | 16-Hb | 1.90 (m) | |
| 17 | 47.1 | 17-H | 1.81 (m) | 18, 21 |
| 18 | 13.0 | 18-H | 0.72 | 14, 17 |
| 19 | 22.9 | 19-H | 1.22 | 1a, 6a, 9 |
| 20 | 36.3 | 20-H | 1.42 (m) | 17, 21, 22a |
| 21 | 17.5 | 21-H | 1.03 (J_{21,20} = 6.5 Hz) | 22 |
| 22 | 33.1 | 22-Ha | 1.34 (m) | 21, 23 |
| | | 22-Hb | 1.80 (m) | |
| 23 | 33.7 | 23-Ha | 2.13 (m) | 22a |
| | | 23-Hb | 2.26 (m) | |
| 24 | 176.6 | | | 22a, 23, 25 |
| 25 | 39.3 | 25-H | 3.35 (J_{25,26} = 7.0 Hz) | 26 |
| 26 | 34.0 | 26-H | 2.59 (J_{26,25} = 7.0 Hz) | 25, 27 |
| 27 | 14.9 | 27-H | 2.10 | 26 |

### Example 6.14 ¹H (600 MHz) and ¹³C (151 MHz) NMR Spectroscopic Data for KDCA-MCYO (Structure Below) in Methanol-d₄

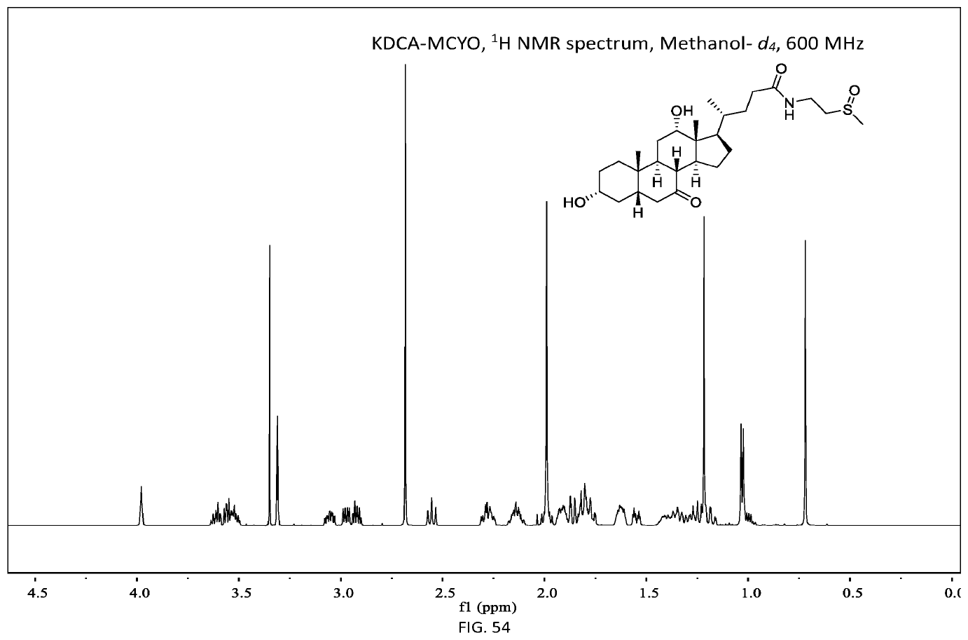

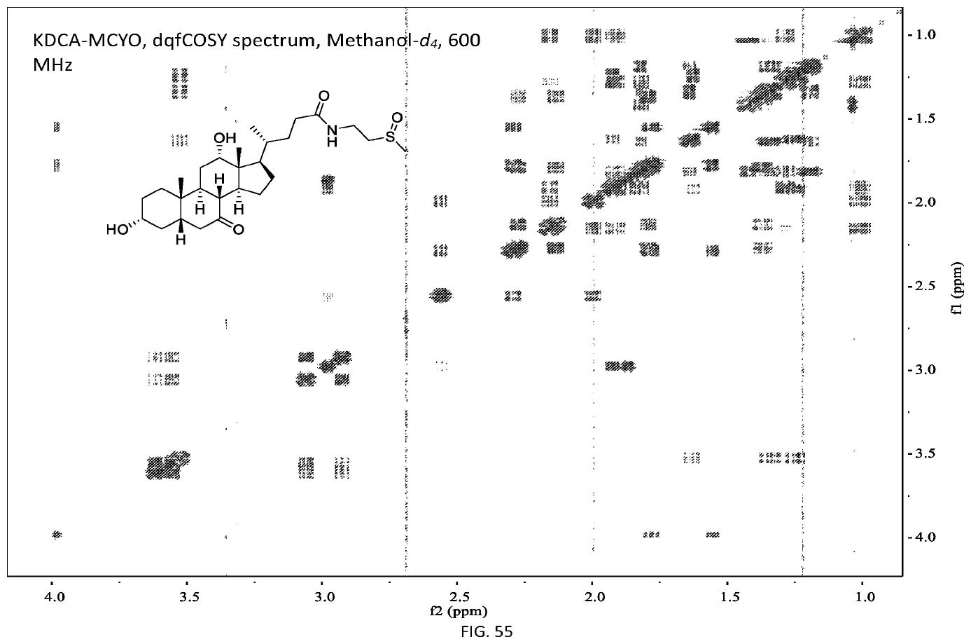

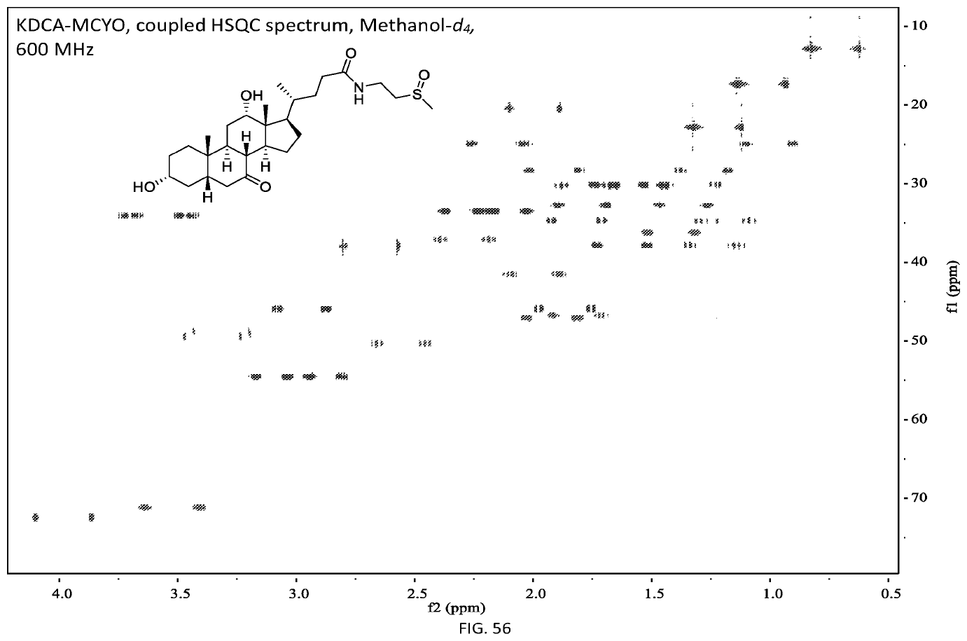

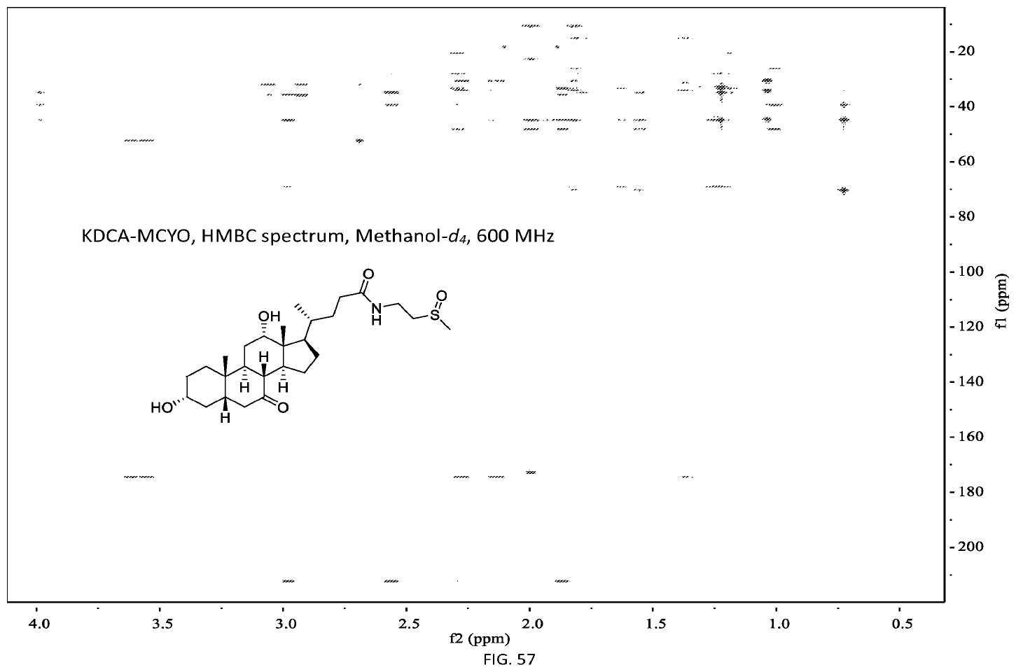

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of KCA-MCYO are shown in FIG. 54 through FIG. 57.

(KDCA-MCYO)

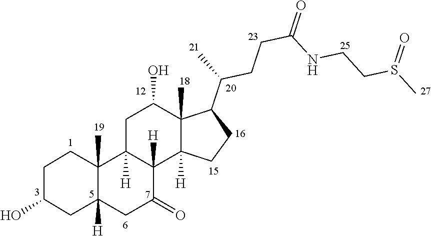

| No. | δc | Proton | δH (J_{HH}[Hz]) | HMBC |
|---|---|---|---|---|
| 1 | 34.8 | 1-Ha | 1.21 (m) | 19 |
| | | 1-Hb | 1.81 (m) | |
| 2 | 30.3 | 2-Ha | 1.33 (m) | |
| | | 2-Hb | 1.80 (m) | |
| 3 | 71.3 | 3-H | 3.52 (m) | 1, 4 |
| 4 | 37.9 | 4-Ha | 1.24 (m) | 6 |
| | | 4-Hb | 1.62 (m) | |
| 5 | 47.2 | 5-H | 1.92 (m) | 1b, 6, 19 |
| 6 | 46.1 | 6-Ha | 1.86 (J_{6a,6b} = 12.7 Hz, J_{6a,5} = 2.3 Hz) | 4a |
| | | 6-Hb | 2.97 (J_{6b,6a} = 12.7 Hz, J_{6b,5} = 5.9 Hz) | |
| 7 | 214.4 | | | 6, 8 |
| 8 | 50.4 | 8-H | 2.55 (J_{8,14} = 11.6 Hz, | 6a, 9, 11a, |
| | | | J_{8,9} = 11.6 Hz) | 14, 15a |
| 9 | 37.2 | 9-H | 2.29 (m) | 1a, 8, 11, 12, 19 |
| 10 | 35.5 | | | 1, 4b, 6a, 9, 19 |
| 11 | 30.3 | 11-Ha | 1.55 (J_{11,12} = Hz, J_{11,9} = Hz) | 9 |
| | | 11-Hb | 1.78 (J_{11,12} = Hz, J_{11,9} = Hz) | |
| 12 | 72.6 | 12-H | 3.99 (J_{12,11} = Hz) | 11a, 17, 18 |
| 13 | 47.2 | | | 11, 12, 14, 17, 18 |
| 14 | 41.6 | 14-H | 1.99 (J_{14,15a} = Hz, J_{14,15b} = Hz, J_{14,8} = Hz) | 8, 12, 15a, 18 |
| 15 | 25.1 | 15-Ha | 1.00 (m) | 14 |
| | | 15-Hb | 2.15 (m) | |
| 16 | 28.4 | 16-Ha | 1.29 (m) | 15a, 17 |
| | | 16-Hb | 1.90 (m) | |
| 17 | 46.9 | 17-H | 1.81 (m) | 18, 21 |
| 18 | 13.0 | 18-H | 0.72 | 14, 17 |
| 19 | 22.9 | 19-H | 1.22 | 1a, 6a, 9 |
| 20 | 36.3 | 20-H | 1.41 (m) | 17, 21, 22a |
| 21 | 17.5 | 21-H | 1.03 (J_{21,20} = 6.5 Hz) | 22 |
| 22 | 32.8 | 22-Ha | 1.34 (m) | 21, 23 |
| | | 22-Hb | 1.80 (m) | |
| 23 | 33.7 | 23-Ha | 2.13 (m) | 22a |
| | | 23-Hb | 2.26 (m) | |
| 24 | 176.8 | | | 22a, 23, 25 |
| 25 | 34.2 | 25-Ha | 3.55 (m) | 26 |
| | | 25-Hb | 3.61 (m) | |
| 26 | 54.6 | 26-Ha | 3.06 (m) | 25, 27 |
| | | 26-Hb | 2.92 (m) | |
| 27 | 38.1 | 27-H | 2.68 | 26 |

### Example 6.15 ¹H (600 MHz) and ¹³C (151 MHz) NMR Spectroscopic Data for CA-MCYO (Structure Below) in Methanol-d₄

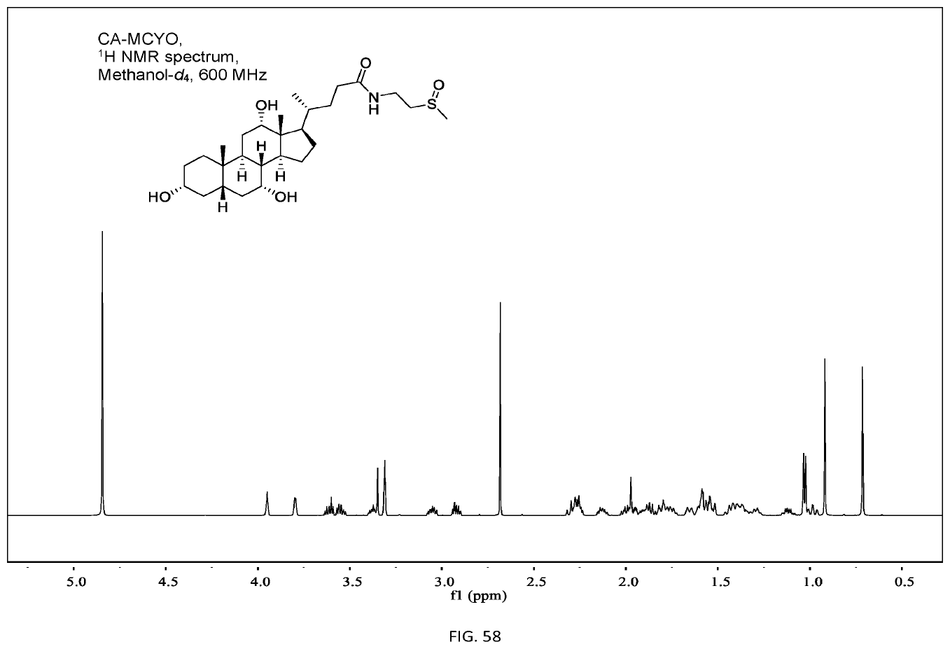

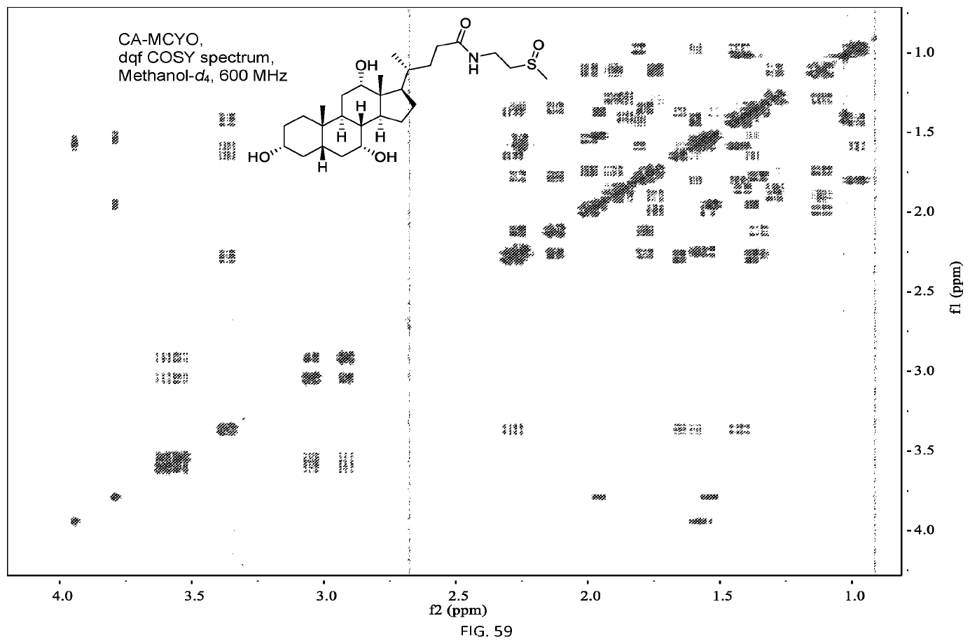

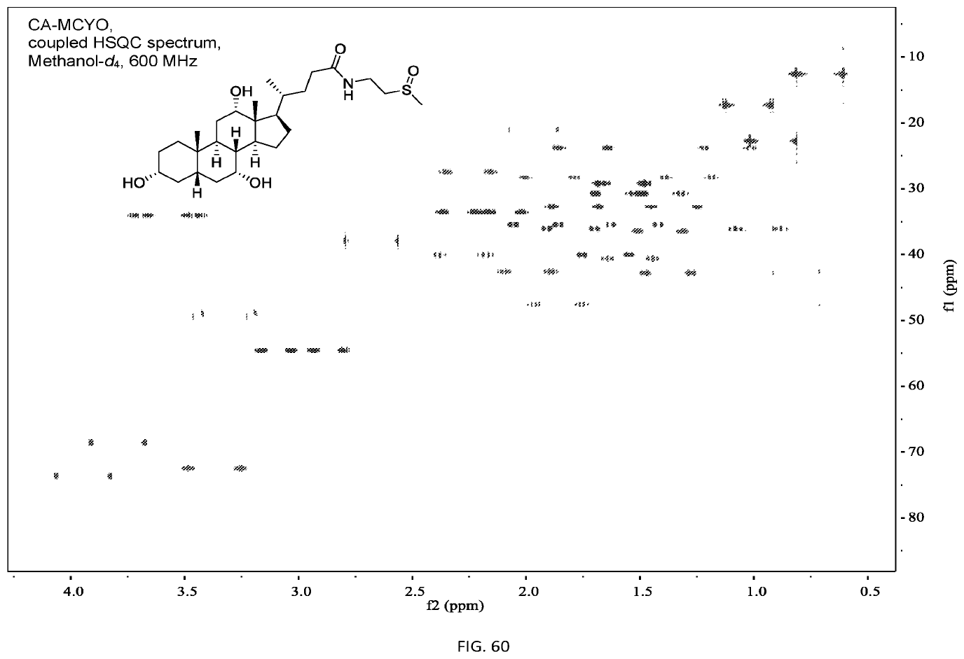

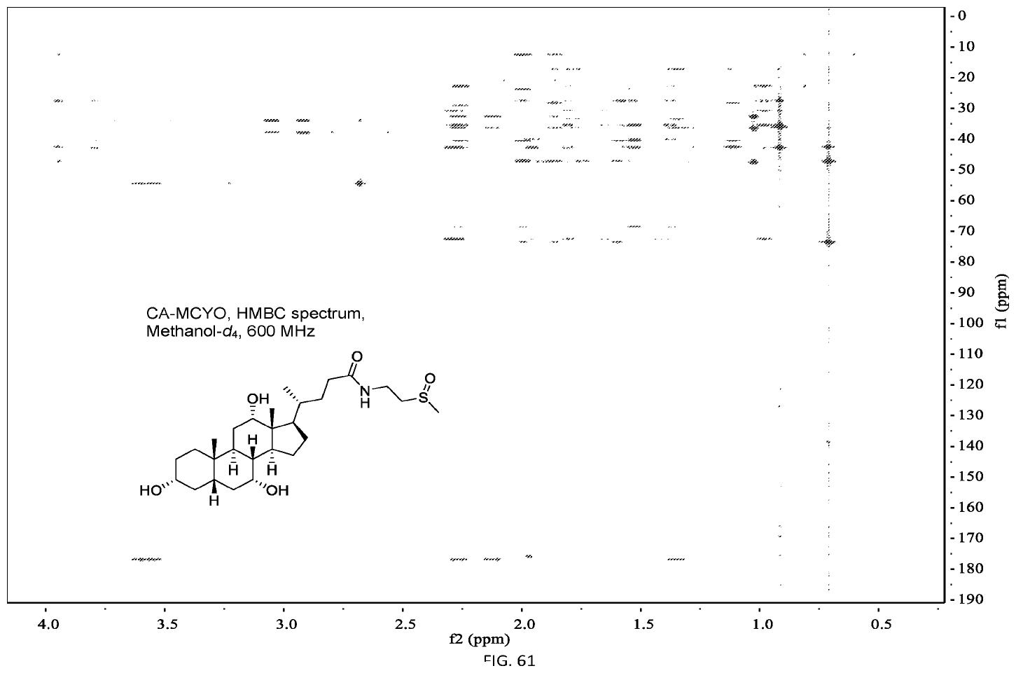

Chemical shifts were referenced to δ(CHD₂OD)=3.31 and δ(¹³CHD₂OD)=49.0. ¹³C chemical determined from the acquired ¹H or dqfCOSY spectra. HMBC correlations are from the proton(s) stated to the indicated ¹³C atom. The corresponding ¹H, dqfCOSY, HSQC and HMBC spectra of KCA-MCYO are shown in FIG. 58 through FIG. 61.

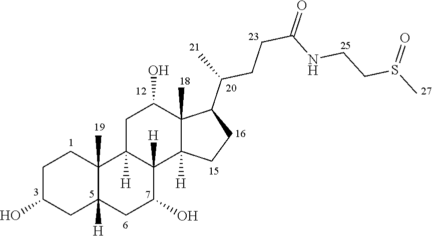

| No. | δc | Proton | δH (J_{HH}[Hz]) | HMBC |
|---|---|---|---|---|
| 1 | 36.2 | 1-Ha | 0.98 (J_{1a,2a} = 3.6 Hz, J_{1a,2b} = 14.1 Hz, J_{1a,1b} = 14.1 Hz) | 19 |
| | | 1-Hb | 1.79 (m) | |
| 2 | 30.9 | 2-Ha | 1.43 (m) | 1a, 4b |
| | | 2-Hb | 1.60 (m) | |
| 3 | 72.5 | 3-H | 3.36 (m) | 1, 4b, 6, 19 |
| 4 | 40.1 | 4-Ha | 1.64 (m) | 6a |
| | | 4-Hb | 2.28 (m) | |
| 5 | 42.8 | 5-H | 1.37 (m) | 1, 4b, 6, 19 |
| 6 | 35.5 | 6-Ha | 1.52 (m) | 4b |
| | | 6-Hb | 1.95 (m) | |
| 7 | 68.6 | 7-H | 3.79 (m) | 6a, 8 |
| 8 | 40.7 | 8-H | 1.55 (m) | 6a, 7, 9, 11, 14, 15a |
| 9 | 27.6 | 9-H | 2.26 (m) | 1a, 7, 8, 11, 12, 14, 19 |
| 10 | 35.7 | | | 1, 4b, 5, 6a, 9, 19 |
| 11 | 29.3 | 11-H | 1.58 (m) | 9 |
| 12 | 73.7 | 12-H | 3.94 (m) | 11, 14, 17, 18 |
| 13 | 47.2 | | | 11, 12, 14, 17, 18 |
| 14 | 42.7 | 14-H | 1.99 (m) | 8, 12, 15a, 18 |
| 15 | 23.9 | 15-Ha | 1.12 (J_{15a,16a} = 4.8 Hz, J_{15a,16b} = 12.0 Hz, J_{15a,15b} = | 14 |
| | | | 12.0 Hz, J_{15a,14} = 12.0 Hz) | |
| | | 15-Hb | 1.74 (m) | |
| 16 | 28.4 | 16-Ha | 1.29 (m) | 15a, 17 |
| | | 16-Hb | 1.89 (m) | |
| 17 | 47.6 | 17-H | 1.86 (m) | 18, 21 |
| 18 | 12.4 | 18-H | 0.72 | 12, 14, 17 |
| 19 | 22.9 | 19-H | 0.92 | 1a, 9 |
| 20 | 36.6 | 20-H | 1.41 (m) | 17, 21, 22a |
| 21 | 16.3 | 21-H | 1.03 (J_{21,20} = 6.5 Hz) | 22a |
| 22 | 32.8 | 22-Ha | 1.34 (m) | 21, 23 |
| | | 22-Hb | 1.78 (m) | |
| 23 | 33.7 | 23-Ha | 2.13 (m) | 22a |
| | | 23-Hb | 2.26 (m) | |
| 24 | 176.9 | | | 22a, 23, 25 |
| 25 | 34.2 | 25-Ha | 3.55 (J_{25a,25b} = 14.1 Hz, J_{25a,26a} = 6.5 Hz, J_{25a,26b} = 6.5 Hz) | 26 |
| | | 25-Hb | 3.61 (J_{25b,25a} = 14.1 Hz, J_{25b,26a} = 6.5 Hz, J_{25b,26b} = 6.5 Hz) | |
| 26 | 54.6 | 26-Ha | 2.92 (J_{26a,25a} = 12.5 Hz, J_{26a,25b} = 6.5 Hz, J_{26a,25b} = 6.5 Hz) | 25, 27 |
| | | 26-Hb | 3.05 (J_{26b,25a} = 12.5 Hz, J_{26b,25a} = 6.5 Hz, J_{26a,25a} = 6.5 Hz) | |
| 27 | 38.1 | 27-H | 2.68 | 26 |

### Example 6.16 ¹H (600 MHz) and ¹³C (151 MHz) NMR Spectroscopic Data for (3MCA-MCY (Structure Below) in Methanol-d₄

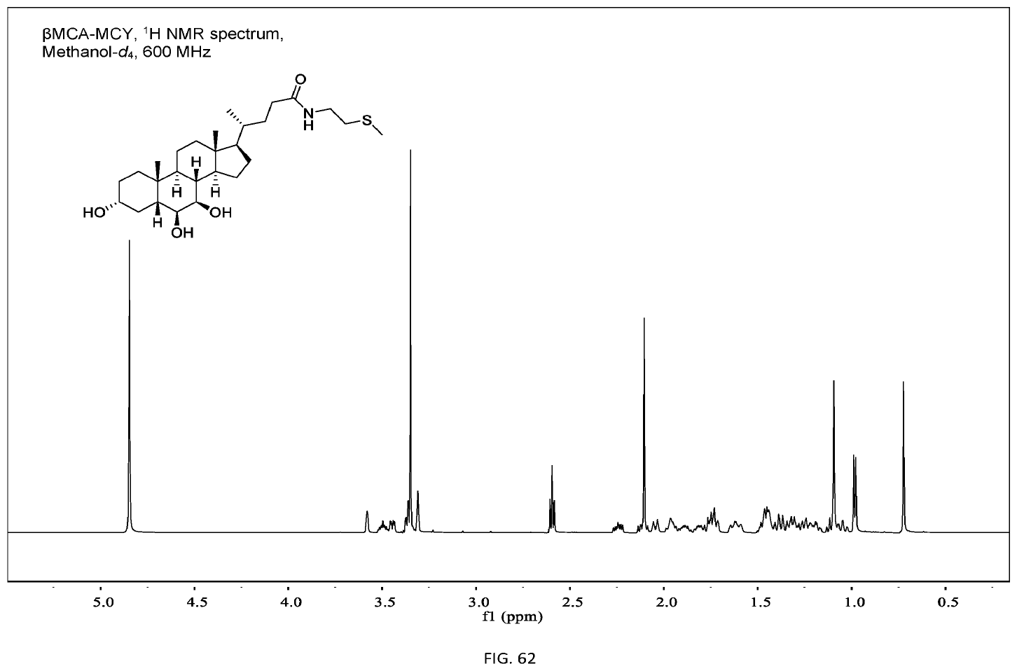

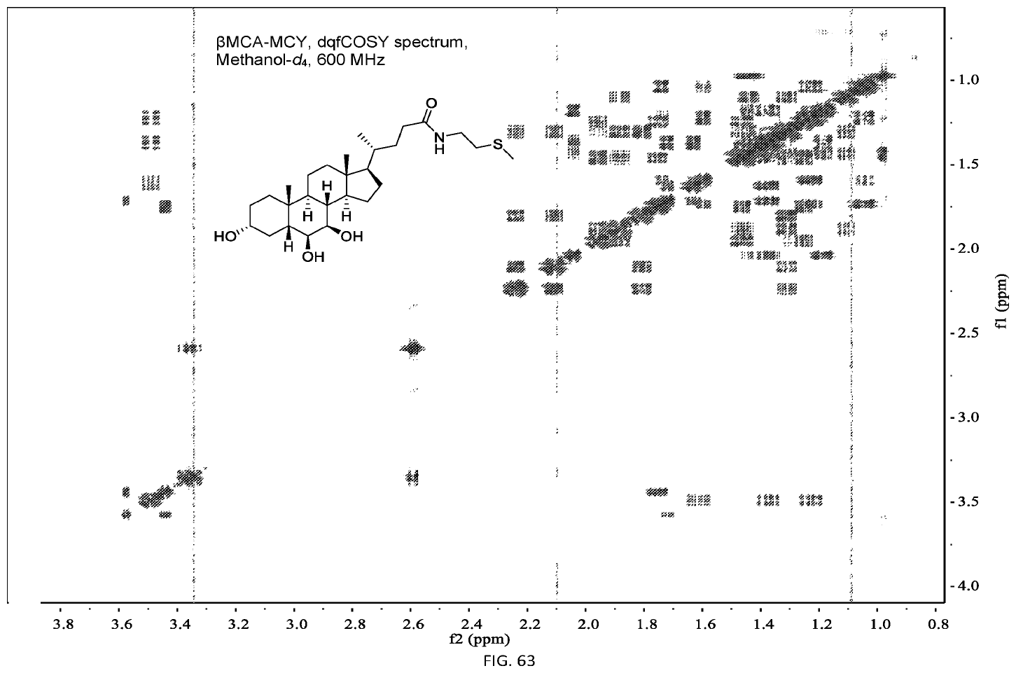

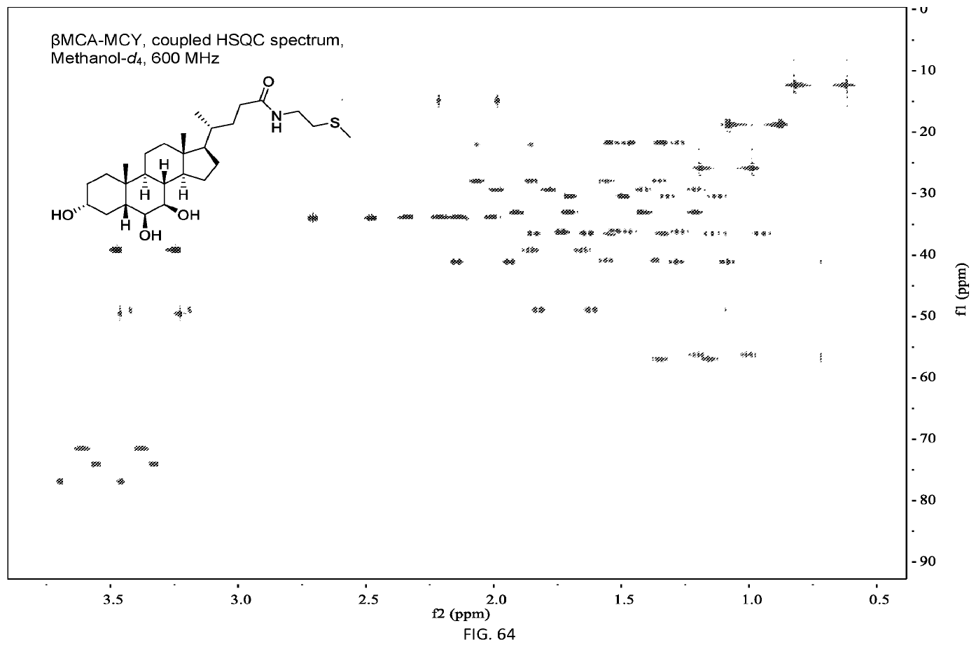

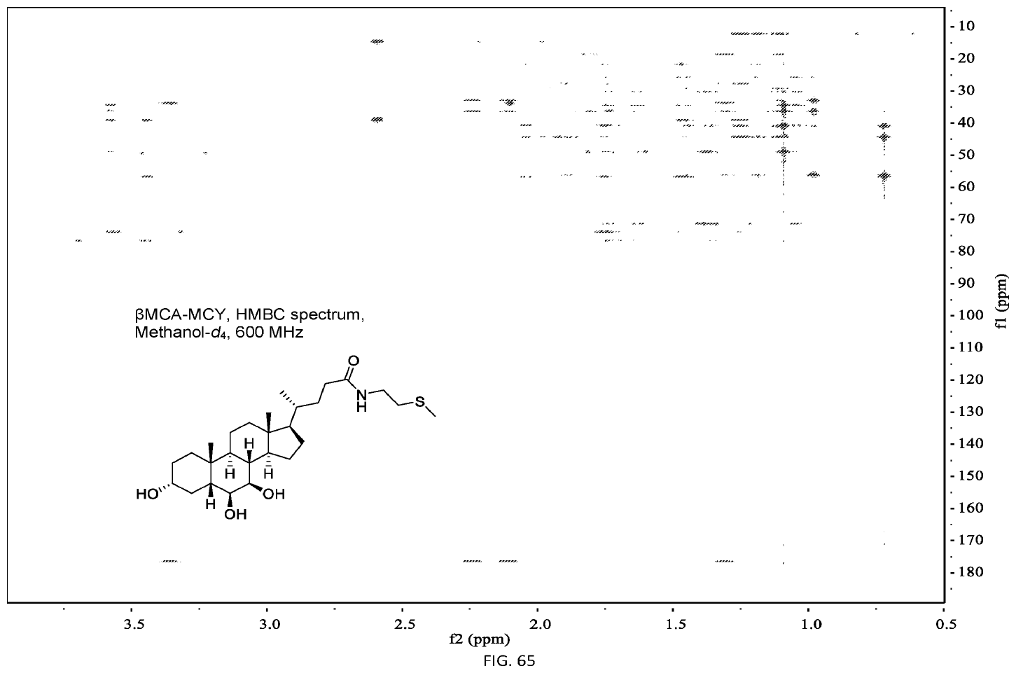

Chemical shifts were referenced to δ(CHD₂OD)=3.31 and δ(¹³CHD₂OD)=49.0. ¹³C chemical shifts were determined via HMBC and HSQC spectra. ¹H, ¹H-J-coupling constants were determined from the acquired ¹H or dqfCOSY spectra. HMBC correlations are from the proton(s) stated to the indicated ¹³C atom. The corresponding ¹H, dqfCOSY, HSQC and HMBC spectra of KCA-MCYO are shown in FIG. 62 through FIG. 65.

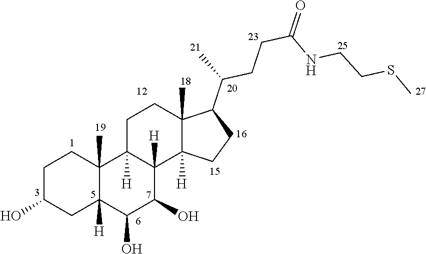

| No. | δc | Proton | δH (J_{HH}[Hz]) | HMBC |
|---|---|---|---|---|
| 1 | 36.5 | 1-Ha | 1.05 (m) | 19 |
| | | 1-Hb | 1.73 (m) | |
| 2 | 30.6 | 2-Ha | 1.22 (m) | 1, 4b |
| | | 2-Hb | 1.60 (m) | |
| 3 | 71.5 | 3-H | 3.49 (m) | 1, 2a, 4b |
| 4 | 36.4 | 4-Ha | 1.38 (m) | 6 |
| | | 4-Hb | 1.63 (m) | |
| 5 | 49.1 | 5-H | 1.72 (m) | 1b, 4b, 6, 19 |
| 6 | 76.9 | 6-H | 3.57 (m) | 4b, 7 |
| 7 | 74.1 | 7-H | 3.44 (J_{7,8} = 10.3 | 6,8 |
| | | | Hz, J_{7,6} = 3.6 Hz) | |
| 8 | 39.4 | 8-H | 1.75 (m) | 6, 7, 9, 11a, 14, 15a |
| 9 | 41.0 | 9-H | 1.46 (m) | 1a, 8, 11a, 12a, 14, 19 |
| 10 | 34.7 | | | 1, 4b, 5, 6, 9, 19 |
| 11 | 21.8 | 11-Ha | 1.32 (m) | 9 |
| | | 11-Hb | 1.50 (m) | |
| 12 | 41.2 | 12-Ha | 1.18 (m) | 11a, 14, 17, 18 |
| | | 12-Hb | 2.04 (m) | |
| 13 | 44.5 | | | 11a, 12, 14, 15b, |
| | | | | 17, 18 |
| 14 | 57.0 | 14-H | 1.24 (m) | 7, 8, 12b, 15a, 18 |
| 15 | 28.1 | 15-Ha | 1.45 (m) | 14, 16b |
| | | 15-Hb | 1.96 (m) | |
| 16 | 29.5 | 16-Ha | 1.31 (m) | 15a, 17 |
| | | 16-Hb | 1.88 (m) | |
| 17 | 56.4 | 17-H | 1.10 (m) | 12a, 16b, 18, 21, 22 |
| 18 | 12.4 | 18-H | 0.72 | 12a, 14, 17 |
| 19 | 25.9 | 19-H | 1.09 | 1a, 5, 9 |
| 20 | 36.7 | 20-H | 1.44 (m) | 17, 21, 22a |
| 21 | 18.9 | 21-H | 0.98 | 17, 22a |
| | | | (J_{21,20} = 6.5 Hz) | |
| 22 | 33.1 | 22-Ha | 1.31 (m) | 21, 23 |
| | | 22-Hb | 1.81 (m) | |
| 23 | 33.9 | 23-Ha | 2.10 (m) | 22a |
| | | 23-Hb | 2.24 (m) | |
| 24 | 176.6 | | | 22a, 23, 25 |
| 25 | 39.3 | 25-H | 3.35 | 26 |
| | | | (J_{25,26} = 7.0 Hz) | |
| 26 | 34.0 | 26-H | 2.58 | 25, 27 |
| | | | (J_{26,25} = 7.0 Hz) | |
| 27 | 14.9 | 27-H | 2.10 | 26 |

### Example 7. Human

It was determined whether these microbiota-dependent metabolites could also be detected in humans. Analyzing human serum samples, almost all microbiota-dependent metabolites identified in mice were identified in humans, including butyrate-MCY, MCYO, and MCYO2. Next, it was determined whether their levels could be altered by dietary intervention in humans. It was determined that upon a brief period of dietary supplementation with inulin fiber, all butyrate-MCY conjugates were significantly increased in the serum (with both mice and humans). Collectively, these data indicate that most, if not all, microbiota-dependent metabolites including MCY conjugates of butyrate are present in human and that levels of these metabolites can be regulated by dietary interventions that influence relevant microbiota communities.

### Example 8. Butyrate-MCY Reduces Resident Treg Levels in Brain and Colon

### Administration of Metabolites

Butyrate-MCY (dissolved in water) was delivered by oral gavage at a rate of 50 mg/kg body weight per day in a volume of 100 μL for two weeks.

Isolation of Cells from Colon and Brain

Mouse colons were removed, cleaned of attached fat tissues, and washed in ice-cold PBS (Sigma-Aldrich). They were then opened longitudinally and washed again in ice-cold PBS. Dissociation of epithelial cells was performed by shaking at 37° C. in HBSS (Sigma-Aldrich) containing 10 mM HEPES, 10 mM EDTA (Thermo Fisher Scientific) and 2 mM dithiothreitol (DTT) two times for 15 min each. After each step, samples were vortexed, and the supernatant containing the epithelial fraction was removed. The tissue was then chopped into 0.5 cm pieces, and enzymatic digestion was performed using collagenase III (1 mg/mL; Worthington), dispase (0.4 U/mL; Thermo Fisher Scientific), DNase I (20 g/mL; Sigma-Aldrich) and 4% FBS for 40 min in a shaker at 37° C. The single cell suspension was then filtered through a 70 m cell strainer and centrifuged through a Percoll (Sigma-Aldrich) gradient and washed. Mesenteric lymph nodes were chopped and incubated in RPMI 1640 medium (Sigma-Aldrich) supplemented with 1% FBS (Sigma-Aldrich), Collagenase II (1 mg/mL; Sigma-Aldrich) and DNaseI (20 g/mL) for 20 min in a shaker at 37° C. Cells were then filtered through a 70 μm cell strainer and washed with RPMI with 5% FBS.

For isolation of brain leukocyte populations, mice were euthanized with CO₂ and perfused through the left ventricle with 20 mL PBS. The whole brain was removed and placed in 2.5 mL digestion buffer (PBS, 5% FCS, 1 mM HEPES) before being finely chopped. Collagenase D (Roche, 400 U) was added to the mixture, which was then incubated at 37° C. for 30 min before adding 50 μL 0.5 M EDTA, followed by a 5-min incubation. Digested tissue was triturated 20 times through a P1000 tip, and then filtered through a 70-μm cell strainer, pelleted at 700 g in a swinging-bucket centrifuge and then resuspended in 10 mL 38% isotonic Percoll and centrifuged at 2,000 r.p.m for 30 min with no brake. The myelin debris layer was removed by aspiration, and the pellet was washed with PBS.

### Intravenous CD45 Labeling

For discriminating between circulating cells and resident brain Treg populations, mice were anesthetized with isoflurane before injection of 1 μg of CD45.2 antibody (clone 104) diluted in sterile PBS into the retroorbital sinus. After 3 min to allow the injected antibody to circulate, mice were euthanized with CO₂ and perfused through the left ventricle with 20 mL PBS. Brain tissue was then harvested and digested as above and stained with the indicated antibodies including pan-CD45 (30-F11) labeled with a separate fluorophore from the i.v. injected CD45.2 antibody. Cells labeled with both the CD45.2 i.v. antibody and pan-CD45 antibody were considered to be circulating, while cells only labeled with the pan-CD45 antibody were considered tissue resident.

### Flow Cytometry

Mouse single-cell suspensions were pre-treated with anti-CD16/32 and then incubated on ice with conjugated antibodies in PBS. Dead cells were excluded with Fixable Aqua Dead Cell Stain (Thermo Fisher). Lineage (Lin) markers used were as follows: Lin1: CD3ε (145-2C11), CD5 (53-7.3), CD8a (53-6.7), Lin2: CD19 (1D3), FcεRI (MAR-1), and CD11c (N418). CD45 (30-F11) and CD4 (GK1.5) were used for surface staining. For brain leukocyte staining, after anti-CD16/32 blocking and incubation with Fixable Aqua Dead Cell Stain, cells were stained with the following antibodies: CD3ε (145-2C11), CD5 (53-7.3), CD8a (53-6.7), CD45 (30-F11), CD4 (GK1.5), and CD69 (H1.F23). Transcription factors were stained in both intestine and brain cells using FoxP3 (FJK-16s) and the eBioscience Foxp3/Transcription Factor Staining Buffer Set (Thermo Fisher).

All antibodies above were used at 1:200 dilutions, except FoxP3 were used at 1:100 dilutions. Stained cells were analyzed on a 5 laser, 18 color custom-configuration BD LSRFortessa (BD). Data were collected using BD FACSDiva version 9.0 and analyzed using FlowJo (version 10.7.1, Tree Star).

### Results

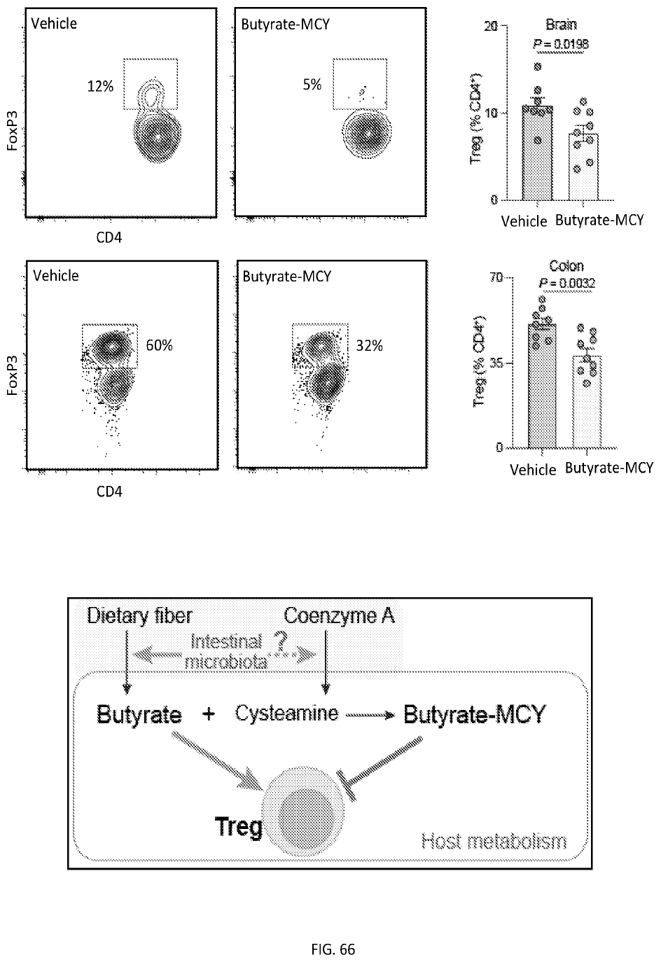

For functional evaluation, the previously unreported MCY conjugates of butyrate were prioritized, given that their microbiota-dependent production is conserved in humans and considering that the corresponding parent metabolite, butyrate, serves important roles in immune homeostasis. Butyrate and other SCFAs have been shown to promote regulatory T cell (Treg) development and expansion (Furusawa, et al. (2013) Nature 504:446-450; Arpaia, et al. (2013) Nature 504:451-455), which has broad implications for resolution of inflammation, host defense, tissue homeostasis, and immune development in both the gut and in distant organs including the CNS (Haghikia, et al. (2015) Immunity 43:817-829; Smith, et al. (2013) Science 341:569-573; Furusawa, et al. (2013) Nature 504:446-450; Borchardt, et al. (1971) Br Med J., 1:205-206; Ayanoglou, et al. (1985) Actual Odontostomatol (Paris) 39:831-840; Fung, et al. (2017) Nat Neurosci 20:145-155; Dombrowski, et al. (2017) Nat Neurosci. 20:674-680). SPF mice were supplemented via oral gavage with butyrate-MCY (50 mg/kg/day, 100 μL/day, 14 days), which was identified in the brain and serum metabolomes and the levels of brain-resident Tregs was measured. Strikingly, upon administration of butyrate-MCY, the percentage of both gut and brain-resident Tregs amongst CD4+ T cells was significantly decreased (FIG. 66). FIG. 66 (top row) provides representative flow cytometry plots of Tregs and quantification of Tregs as percentage of all CD4+ T cells in brain tissue from vehicle or butyrate-MCY treated mice. FIG. 66 (bottom row) provides representative flow cytometry plots of Tregs (Live CD45+CD3+CD4+FoxP3+) and quantification of Tregs as percentage of all CD4+ T cells in colon tissue from vehicle or butyrate-MCY treated mice. n=8-9 mice per condition. Data are mean±s.e.m. P values were calculated by unpaired two-sided t-test with Welch's correction. FIG. 66 (bottom) also provides a schematic of a model, without being bound by theory, for the regulation of Treg levels via microbial SCFA metabolism. Gut microbiota-derived butyrate promotes Treg differentiation, whereas butyrate-MCY, derived from host-dependent conjugation of butyrate with cysteamine, suppresses Treg differentiation.

Additionally, intravenous CD45 labeling was performed and levels of the resident Treg marker CD69 was measured to confirm that the measured Tregs were brain-resident (Liston, et al. (2022) Immunol Lett 248:26-30; Anderson, et al. (2014) Nat Protoc 9:209-222). Together these data indicate that the MCY conjugate of butyrate counteracts the effects of free butyrate on Tregs both locally in the intestine and at distant peripheral tissue sites including the CNS.

Thus, butyrate-MCY reduces Treg levels significantly in both the gut and brain. Notably, Treg-reducing activity of butyrate-MCY was at concentrations orders of magnitude below those required to increase Treg levels using free butyrate. Given the known roles for Tregs in the control and resolution of CNS autoimmunity (Lafaille, et al. (1994) Cell 78:399-408; Goverman, et al. (2021) N Engl J Med 384:578-580), tissue repair (Shi, et al. (2021) Immunity 54:1527-1542; Wang, et al. (2022) Acta Pharmacol Sin 43:1-9), and maturation of microglia (Pasciuto, et al. (2020) Cell 182:625-640), these findings indicate that the gut microbiota and their metabolites play critical and bidirectional roles in shaping brain Treg responses and their downstream contribution to CNS function in both pathological and steady-state contexts.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method for improving the health of an animal, the method comprising the step of administering to the animal an effective amount of a therapeutic composition comprising a cysteamide of a carboxylic acid.

2: The method of claim 1, wherein the cysteamide of a carboxylic acid comprises a compound of Formula I: 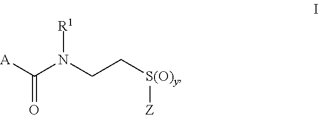
wherein:
A is selected from —H and an optionally substituted C₁₋₃₉ aliphatic group;
R¹ is selected from —H, and an optionally substituted C₁₋₄₀ aliphatic group;
Z is —H, or an optionally substituted C₁₋₄₀ aliphatic group; and
y is 0, 1, or 2.

3: The method of claim 2, wherein the moiety A is derived from an endogenous carboxylic acid.

4: The method of claim 3, wherein the endogenous carboxylic acid comprises a short-chain fatty acid.

5: The method of claim 3, wherein the endogenous carboxylic acid comprises a bile acid.

6: The method of claim 4, wherein the short chain fatty acid is selected from propionic acid, butyric acid, and valeric acid.

7: The method of claim 5, wherein the short chain fatty acid comprises butyric acid.

8: The method of claim 2, wherein R¹ is —H.

9: The method of claim 2, wherein Z is other than —H.

10: The method of claim 9, wherein Z is an optionally substituted moiety selected from the group consisting of: a C₁₋₂₄ alkyl group, a C₁₋₁₂ alkyl group, a C₁₋₈ alkyl group, a C₁₋₆ alkyl group, a C₁₋₅ alkyl group, a C₁₋₄ alkyl group, a C₁₋₃ alkyl group, a C₁₋₂ alkyl group, and a methyl group.

11: The method of claim 9, wherein Z is methyl.

12: The method of claim 2, wherein y is 0.

13: The method of claim 2, wherein y is 1 or 2.

14: The method of claim 2, wherein the composition comprises a mixture of two or more compounds that differ only in the value of y.

15: A method for improving the health of an animal, the method comprising the step of administering to the animal an effective amount of a therapeutic composition comprising an S-methylcysteamide of a carboxylic acid.

16: A method for improving the health of an animal, the method comprising the step of administering to the animal an effective amount of a therapeutic composition comprising a sulfoxide or sulfone of an S-methylcysteamide of a carboxylic acid.

17: The method of claim 1, wherein the improved health comprises a reduction in symptoms from a disorder associated with immunomodulation.

18: The method of claim 1, wherein the improved health comprises a reduction in symptoms from an autoimmune disease.

19: The method of claim 1, wherein the improved health comprises a reduction in symptoms from an infectious disease.

20: The method of claim 1, wherein the improved health comprises a reduction in symptoms from a mental health disorder.

21: The method of claim 1, wherein the improved health comprises prevention or treatment of cancer or a proliferative disorder.

22: A method of modulating the bioavailability or distribution of a biologically active carboxylic acid in an animal in need of treatment with said carboxylic acid, the method comprising administering to the animal a composition comprising a cysteamide of the carboxylic acid.

23: The method of claim 22, wherein the cysteamide is an S-methyl cysteamide.

24: The method of claim 22, wherein the cysteamide comprises a sulfoxide or sulfone.

25: A composition of matter comprising the S-methyl cysteamide of a carboxylic acid.

26: The composition of claim 25 comprising a substantially pure sample of the S-methyl cysteamide of a carboxylic acid.

27: The composition of claim 26, characterized in that the sample contains a mass of at least 1 g at least 10 g, at least 50 g, at least 100 g, at least 500 g, or at least 1 kg.

28: The composition of claim 25, characterized in that the S-methyl cysteamide of the carboxylic acid is produced via a manufacturing process.

29: The composition of claim 28, wherein the manufacturing process comprises chemical synthesis.

30: The composition of claim 28, wherein the manufacturing process comprises fermentation.

31: The composition of claim 28, wherein the manufacturing process comprises purification of the S-methyl cysteamide.

32: An isolated compound comprising an S-methylcysteamide of a short chain carboxylic acid.

33: The isolated compound of claim 32, selected from the group consisting of:

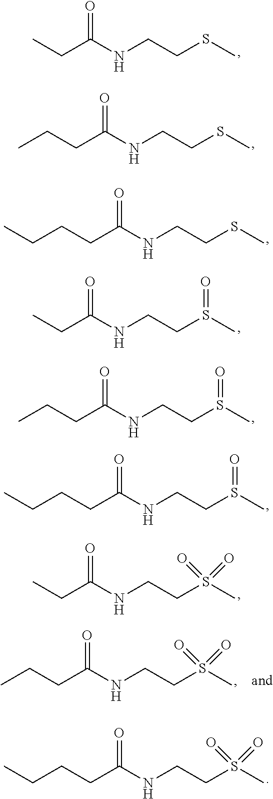

34: A method for enhancing the ability of a therapeutically active carboxylic acid to cross the blood brain barrier of an animal in need of such therapy, the method comprising treating the animal with an S-methyl cysteamide of the carboxylic acid.

35: A method for reducing, suppressing, and/or inhibiting regulatory T cells (Treg) of an animal, the method comprising the step of administering to the animal an effective amount of a therapeutic composition comprising a cysteamide of a carboxylic acid.

36: The method of claim 35, wherein said cysteamide is an S-methylcysteamide of a short chain carboxylic acid.

37: The method of claim 35, wherein said cysteamide is butyrate-methylcysteamide (MCY).
